SEQUENCE LISTING (91 sequences)

SEQ ID NO: 1 - protein, 33 aa, synthetic construct
RMKQLEDEIEELESENYHLENRIARLRKRIGER

SEQ ID NO: 2 - protein, 33 aa, synthetic construct
RMKQLEDEVEELESENYHLENRIARLRKRIGER

SEQ ID NO: 3 - protein, 33 aa, synthetic construct
RSKQLEDEVEELESENYHLENRVARLRKRVGER

SEQ ID NO: 4 - protein, 33 aa, synthetic construct
RMKQLEDEVEELESENYHLENRVARLRKRVGER

SEQ ID NO: 5 - protein, 33 aa, synthetic construct
RMKQLEDKIEELLSKIYHLENEIARLKKLIGER

SEQ ID NO: 6 - protein, 33 aa, synthetic construct
RSKQLEDEVEELESENYHLENRVAALRKRVGER

SEQ ID NO: 7 - protein, 33 aa, synthetic construct
RMKQLEDEIEELESEIYHLENRIARLRKRIGER

SEQ ID NO: 8 - protein, 43 aa, synthetic construct
LEIEAAFLEQENTALETEVAELEQEVQRLENIVSQYETRYGPL

SEQ ID NO: 9 - protein, 43 aa, synthetic construct
LEIEAAFLEQENTALETEVAELEQEVQRLENEVSQYETRYGPL

SEQ ID NO: 10 - protein, 30 aa, synthetic construct
AQLEKELQALEKENAQLEWELQALEKELAQ

SEQ ID NO: 11 - protein, 37 aa, synthetic construct
AQLEKELQALEKENAQLEWELQALEKELAQLEKELQA

SEQ ID NO: 12 - protein, 44 aa, synthetic construct
AQLEKELQALEKENAQLEWELQALEKELAQLEKELQALEKELAQ

SEQ ID NO: 13 - protein, 30 aa, synthetic construct
AQLEKELQALEKKLAQLEWENQALEKELAQ

SEQ ID NO: 14 - protein, 37 aa, synthetic construct
AQLEKELQALEKKLAQLEWELQALEKENAQLEKELQA

SEQ ID NO: 15 - protein, 30 aa, synthetic construct
AQLEKEVQALEKKVAQLEWENQALEKEVAQ

SEQ ID NO: 16 - protein, 30 aa, synthetic construct
AQSEKEVQALEKKVAQLEWENQALEKEVAQ

SEQ ID NO: 17 - protein, 29 aa, synthetic construct
AQSEKEVQALEKKVAQLEWENQALEKESA

SEQ ID NO: 18 - protein, 44 aa, synthetic construct
AQLEKELQALEKKLAQLEWENQALEKELAQLEKELQALEKELAQ

SEQ ID NO: 19 - protein, 58 aa, synthetic construct
AQLEKELQALEKKLAQLEWENQALEKELAQLEKELQALEKELAQLEKELQALEKELAQ

SEQ ID NO: 20 - protein, 82 aa, synthetic construct
LRKIKETVDQVEELRRALRIKSQELEVKNAAANDKLKKMVKDQQEAEKKKVMSQEIQEQLHKQQEVIADKQMSVKEDLDKVE

SEQ ID NO: 21 - protein, 33 aa, synthetic construct
RMKQLRDRIEELRSRNYHLRNEIARLEKEIGER

SEQ ID NO: 22 - protein, 33 aa, synthetic construct
RMKQLRDRVEELRSRNYHLRNEIARLEKEIGER

SEQ ID NO: 23 - protein, 33 aa, synthetic construct
RSKQLRDRVEELRSRNYHLRNEVARLEKEVGER

SEQ ID NO: 24 - protein, 33 aa, synthetic construct
RMKQLRDRVEELRSRNYHLRNEVARLEKEVGER

SEQ ID NO: 25 - protein, 33 aa, synthetic construct; identical to SEQ ID NO: 5

SEQ ID NO: 26 - protein, 33 aa, synthetic construct
RSKQLRDRVEELRSRNYHLRNEVAALEKEVGER

SEQ ID NO: 27 - protein, 33 aa, synthetic construct
RMKQLRDRIEELRSRIYHLRNEIARLEKEIGER

SEQ ID NO: 28 - protein, 43 aa, synthetic construct
LEIRAAFLRRRNTALRTRVAELRQRVQRLRNIVSQYETRYGPL

SEQ ID NO: 29 - protein, 43 aa, synthetic construct
LEIRAAFLRRRNTALRTRVAELRQRVQRARNRVSQYRTRYGPL

SEQ ID NO: 30 - protein, 30 aa, synthetic construct
AQLKKKLQALKKKNAQLKWKLQALKKKLAQ

SEQ ID NO: 31 - protein, 37 aa, synthetic construct
AQLKKKLQALKKKNAQLKWKLQALKKKLAQLKKKLQA

SEQ ID NO: 32 - protein, 44 aa, synthetic construct
AQLKKKLQALKKKNAQLKWKLQALKKKLAQLKKKLQALKKKLAQ

SEQ ID NO: 33 - protein, 30 aa, synthetic construct
AQLKKKLQANKKELAQLKWKLQALKKKLAQ

SEQ ID NO: 34 - protein, 37 aa, synthetic construct
QALKKKLAQNKKKLQALKKELAQLKWKLQALKKKLAQ

SEQ ID NO: 35 - protein, 30 aa, synthetic construct
AQVKKKLQANKKELAQVKWKLQAVKKKLAQ

SEQ ID NO: 36 - protein, 30 aa, synthetic construct
AQVKKKLQANKKELAQVKWKLQAVKKKSAQ

SEQ ID NO: 37 - protein, 30 aa, synthetic construct
AQSKKKLQANKKELAQVKWKLQAVKKKSAQ

SEQ ID NO: 38 - protein, 44 aa, synthetic construct
AQLKKKLQALKKKLAQLKKKLQANKKELAQLKWKLQALKKKLAQ

SEQ ID NO: 39 - protein, 58 aa, synthetic construct
AQLKKKLQALKKKLAQLKKKLQALKKKLAQLKKKLQANKKELAQLKWKLQALKKKLAQ

SEQ ID NO: 40 - protein, 82 aa, synthetic construct
LRNELQKLEDDAKDNQQKANEVEQMIRDLEASIARYKEEYAVLISEAQAIKADLAAVEAKVNRSTALLKSLSAERERWEKTS

SEQ ID NO: 41 - protein, 30 aa, synthetic construct
GGGGGGATNFSLLKLAGDVELNAGAGGGGG

SEQ ID NO: 42 - protein, 40 aa, synthetic construct
GGGGGGGGGGGATNFSLLKLAGDVELNAGAGGGGGGGGGG

SEQ ID NO: 43 - protein, 60 aa, synthetic construct
GGGSGGGSGGGSGGGSGGGSGATNFSLLKLAGDVELNAGAGGGSGGGSGGGSGGGSGGGS

SEQ ID NO: 44 - protein, 6 aa, synthetic construct
GGSSGG

SEQ ID NO: 45 - protein, 5 aa, synthetic construct
GGGGG

SEQ ID NO: 46 - protein, 22 aa, synthetic construct
GGSRDHMVLHEYVNAAGITSGG

SEQ ID NO: 47 - protein, 70 aa, synthetic construct
MAYTPGSRGGRGGSRGGRGGFNGGRGGFGGGRGGARGGGRGGARGGRGGRGGARGGRGGSSGGRGGAKGG

SEQ ID NO: 48 - protein, 12 aa, synthetic construct
GGGGGSSGGGGG

SEQ ID NO: 49 - protein, 38 aa, synthetic construct
GGRQLLAEKRELEEKKRREEEKKREEEERERERAQRGG

SEQ ID NO: 50 - protein, 25 aa, synthetic construct
GGKIAALKEKIAALKEKIAALKEGG

SEQ ID NO: 51 - protein, 21 aa, synthetic construct
EIAALEKEIAALEKEIAALEK

SEQ ID NO: 52 - protein, 15 aa, synthetic construct
GGVSGWRLFKKISGG

SEQ ID NO: 53 - protein, 7 aa, synthetic construct
AANDENY

SEQ ID NO: 54 - protein, 5 aa, synthetic construct
MEEVF

SEQ ID NO: 55 - protein, 5 aa, synthetic construct
MEEVW

SEQ ID NO: 56 - protein, 7 aa, synthetic construct
ENLYFQS

SEQ ID NO: 57 - protein, 104 aa, synthetic construct
GGGGRMKQLEDEIEELESENYHLENRIARLRKRIGERGGGGGGATNFSLLKLAGDVELNAGAGGGGGRMKQLRDRIEELRSRNYHLRNEIARLEKEIGERGGGG

SEQ ID NO: 58 - protein, 104 aa, synthetic construct
GGGGRMKQLEDEVEELESENYHLENRIARLRKRIGERGGGGGGATNFSLLKLAGDVELNAGAGGGGGRMKQLRDRIEELRSRNYHLRNEIARLEKEIGERGGGG

SEQ ID NO: 59 - protein, 104 aa, synthetic construct
GGGGRMKQLEDEVEELESENYHLENRIARLRKRIGERGGGGGGATNFSLLKLAGDVELNAGAGGGGGRMKQLRDRVEELRSRNYHLRNEIARLEKEIGERGGGG

SEQ ID NO: 60 - protein, 104 aa, synthetic construct
GGGGRSKQLEDEVEELESENYHLENRVARLRKRVGERGGGGGGATNFSLLKLAGDVELNAGAGGGGGRSKQLRDRVEELRSRNYHLRNEVARLEKEVGERGGGG

SEQ ID NO: 61 - protein, 104 aa, synthetic construct
GGGGRMKQLEDEVEELESENYHLENRVARLRKRVGERGGGGGGATNFSLLKLAGDVELNAGAGGGGGRSKQLRDRVEELRSRNYHLRNEVARLEKEVGERGGGG

SEQ ID NO: 62 - protein, 104 aa, synthetic construct
GGGGRMKQLEDEVEELESENYHLENRVARLRKRVGERGGGGGGATNFSLLKLAGDVELNAGAGGGGGRMKQLRDRVEELRSRNYHLRNEVARLEKEVGERGGGG

SEQ ID NO: 63 - protein, 114 aa, synthetic construct
GGGGRMKQLEDKIEELLSKIYHLENEIARLKKLIGERGGGGGGGGGGGATNFSLLKLAGDVELNAGAGGGGGGGGGGRMKQLEDKIEELLSKIYHLENEIARLKKLIGERGGGG

SEQ ID NO: 64 - protein, 104 aa, synthetic construct
GGGGRSKQLEDEVEELESENYHLENRVAALRKRVGERGGGGGGATNFSLLKLAGDVELNAGAGGGGGRSKQLRDRVEELRSRNYHLRNEVAALEKEVGERGGGG

SEQ ID NO: 65 - protein, 104 aa, synthetic construct
GGGGRMKQLEDEIEELESEIYHLENRIARLRKRIGERGGGGGGATNFSLLKLAGDVELNAGAGGGGGRMKQLRDRIEELRSRIYHLRNEIARLEKEIGERGGGG

SEQ ID NO: 66 - protein, 154 aa, synthetic construct
GGGGLEIEAAFLEQENTALETEVAELEQEVQRLENIVSQYETRYGPLGGGSGGGSGGGSGGGSGGGSGATNFSLLKLAGDVELNAGAGGGSGGGSGGGSGGGSGGGSLEIRAAFLRRRNTALRTRVAELRQRVQRLRNIVSQYETRYGPLGGGG

SEQ ID NO: 67 - protein, 154 aa, synthetic construct
GGGGLEIEAAFLEQENTALETEVAELEQEVQRLENEVSQYETRYGPLGGGSGGGSGGGSGGGSGGGSGATNFSLLKLAGDVELNAGAGGGSGGGSGGGSGGGSGGGSLEIRAAFLRRRNTALRTRVAELRQRVQRARNRVSQYRTRYGPLGGGG

SEQ ID NO: 68 - protein, 98 aa, synthetic construct
GGGGAQLEKELQALEKENAQLEWELQALEKELAQGGGGGGATNFSLLKLAGDVELNAGAGGGGGAQLKKKLQALKKKNAQLKWKLQALKKKLAQGGGG

SEQ ID NO: 69 - protein, 142 aa, synthetic construct
GGGGAQLEKELQALEKENAQLEWELQALEKELAQLEKELQAGGGSGGGSGGGSGGGSGGGSGATNFSLLKLAGDVELNAGAGGGSGGGSGGGSGGGSGGGSAQLKKKLQALKKKNAQLKWKLQALKKKLAQLKKKLQAGGGG

SEQ ID NO: 70 - protein, 156 aa, synthetic construct
GGGGAQLEKELQALEKENAQLEWELQALEKELAQLEKELQALEKELAQGGGSGGGSGGGSGGGSGGGSGATNFSLLKLAGDVELNAGAGGGSGGGSGGGSGGGSGGGSAQLKKKLQALKKKNAQLKWKLQALKKKLAQLKKKLQALKKKLAQGGGG

SEQ ID NO: 71 - protein, 74 aa, synthetic construct
GGGGAQLEKELQALEKKLAQLEWENQALEKELAQGGSSGGAQLKKKLQANKKELAQLKWKLQALKKKLAQGGGG

SEQ ID NO: 72 - protein, 88 aa, synthetic construct
GGGGAQLEKELQALEKKLAQLEWELQALEKENAQLEKELQAGGSSGGQALKKKLAQNKKKLQALKKELAQLKWKLQALKKKLAQGGGG

SEQ ID NO: 73 - protein, 74 aa, synthetic construct
GGGGAQLEKEVQALEKKVAQLEWENQALEKEVAQGGSSGGAQVKKKLQANKKELAQVKWKLQAVKKKLAQGGGG

SEQ ID NO: 74 - protein, 74 aa, synthetic construct
GGGGAQSEKEVQALEKKVAQLEWENQALEKEVAQGGSSGGAQVKKKLQANKKELAQVKWKLQAVKKKSAQGGGG

SEQ ID NO: 75 - protein, 74 aa, synthetic construct
GGGGAQSEKEVQALEKKVAQLEWENQALEKESAQGGSSGGAQSKKKLQANKKELAQVKWKLQAVKKKSAQGGGG

SEQ ID NO: 76 - protein, 102 aa, synthetic construct
GGGGAQLEKELQALEKKLAQLEWENQALEKELAQLEKELQALEKELAQGGSSGGAQLKKKLQALKKKLAQLKKKLQANKKELAQLKWKLQALKKKLAQGGGG

SEQ ID NO: 77 - protein, 130 aa, synthetic construct
GGGGAQLEKELQALEKKLAQLEWENQALEKELAQLEKELQALEKELAQLEKELQALEKELAQGGSSGGAQLKKKLQALKKKLAQLKKKLQALKKKLAQLKKKLQANKKELAQLKWKLQALKKKLAQGGGG

SEQ ID NO: 78 - protein, 177 aa, synthetic construct
GGGGLRKIKETVDQVEELRRALRIKSQELEVKNAAANDKLKKMVKDQQEAEKKKVMSQEIQEQLHKQQEVIADKQMSVKEDLDKVEGGGGGLRNELQKLEDDAKDNQQKANEVEQMIRDLEASIARYKEEYAVLISEAQAIKADLAAVEAKVNRSTALLKSLSAERERWEKTSGGGG

SEQ ID NO: 79 - protein, 194 aa, synthetic construct
GGGGLRKIKETVDQVEELRRALRIKSQELEVKNAAANDKLKKMVKDQQEAEKKKVMSQEIQEQLHKQQEVIADKQMSVKEDLDKVEGGSRDHMVLHEYVNAAGITSGGLRNELQKLEDDAKDNQQKANEVEQMIRDLEASIARYKEEYAVLISEAQAIKADLAAVEAKVNRSTALLKSLSAERERWEKTSGGGG

SEQ ID NO: 80 - protein, 104 aa, synthetic construct
GGGGAQLEKELQALEKKLAQLEWELQALEKENAQLEKELQAGGSRDHMVLHEYVNAAGITSGGQALKKKLAQNKKKLQALKKELAQLKWKLQALKKKLAQGGGG

SEQ ID NO: 81 - DNA, 101 bp, synthetic construct
  source 1..101: mol_type = other DNA
aacttctcaatcaacacgctgccatccgtggtggatcacgagatcacatggttctgcacgagtacgttaacgccgcgggaatcacctcgggcggagaagaa

SEQ ID NO: 82 - DNA, 673 bp, synthetic construct
  source 1..673: mol_type = other DNA
  misc_feature 617: note = Silent Mutation
aacttctcaatcaacacgctgccatccgtggcggcggcggcctgagaaagataaaagagactgtcgatcaagtagaagagctcagaagagctcttcgtatcaaatcgcaagagcttgaagtaaaaaatgcagcggcaaacgacaaactcaagaagatggtgaaggaccaacaagaggccgaaaaaaaaaaagtaatgtcacaagaaattcaagagcagcttcataaacaacaggaagtcatcgctgacaagcagatgtcagtgaaagaggatttagacaaggtagagggtggatcacgagatcacatggttctgcacgagtacgttaacgccgcgggaatcacctcgggcggactgcgtaacgagcttcagaagttggaggacgatgctaaagacaaccaacaaaaagctaacgaagtagaacagatgatacgtgacttagaagccagtatagcgcgatacaaagaggagtatgctgttttgatctcagaggcgcaggctataaaggcagacctggcggcggtcgaggcgaaagttaatagaagtacagcccttcttaaatctcttagtgccgagcgtgagcgatgggagaaaacgagtggtggaggtggagaagaaattgacggatacgctgaggattacaagaagatgcgtgcaatgggagatcgtgtcac

SEQ ID NO: 83 - DNA, 403 bp, synthetic construct
  source 1..403: mol_type = other DNA
  misc_feature 347: note = Silent Mutation
aacttctcaatcaacacgctgccatccgtggtggtggcggagcgcaattggaaaaggaacttcaggcactggaaaaaaagctggcgcaacttgagtgggagctccaagctctggagaaagagaatgcccagctggaaaaggagctccaagctggtggatcacgagatcacatggttctgcacgagtacgttaacgccgcgggaatcacctcgggcggacaggctctgaagaagaagttggcgcagaacaagaagaaactccaagcgttaaagaaagagcttgcacagttaaaatggaagcttcaagcgctcaagaagaaattagcccaaggtggcggcggagaagaaattgacggatacgctgaggattacaagaagatgcgtgcaatgggagatcgtgtcac

SEQ ID NO: 84 - protein, 213 aa, synthetic construct
SKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEK

SEQ ID NO: 85 - protein, 159 aa, synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 86 - protein, 151 aa, synthetic construct
MSYYHHHHHHDYDIPTTENLYFQGSKQALKEKELGNDAYKKKDFDTALKHYDKAKELDPTNMYILNQAAVYFEKGDYNKCRELCEKAIEVGRENREDYRLIAIAYARIGNSYFKEEKYKDAIHFYNKSLAEHRTPKVLKKCQQAEKILKEQ

SEQ ID NO: 87 - protein, 151 aa, synthetic construct
MSYYHHHHHHDYDIPTTENLYFQGSKQALKEKELGNDAYKKKDFDTALKHYDKAKELDPTNMYIMNQAAVYFEKGDYNKCRELCEKAIEVGRENREDYRMIAYAYARIGNSYFKEEKYKDAIHFYNKSLAEHRTPKVLKKCQQAEKILKEQ

SEQ ID NO: 88 - protein, 151 aa, synthetic construct
MSYYHHHHHHDYDIPTTENLYFQGSKQALKEKELGNDAYKKKDFDTALKHYDKAKELDPTNMYIMNQAAVYFEKGDYNKCRELCEKAIEVGRENREDYRMIAYAYADIGDSYFKEEKYKDAIHFYNKSLAEHRTPKVLKKCQQAEKILKEQ

SEQ ID NO: 89 - protein, 128 aa, synthetic construct
MKQALKEKELGNDAYKKKDFDTALKHYDKAKELDPTNMTYIINQAAVYFEKGDYNKCRELCEKAIEVGRENREDYRWIAIAYARIGNSYFKEEKYKDAIHFYNKSLAEHRTPKVLKKCQQAEKILKEQ

SEQ ID NO: 90 - protein, 108 aa, synthetic construct
SQLTPRRPYLLRAFYEWLLDNQLTPHLVVDVTLPGVQVPMEYARDGQIVLNIAPRAVGNLELANDEVRFNARFGGIPRQVSVPLAAVLAIYARENGAGTMFEPEAAYD

SEQ ID NO: 91 - protein, 4 aa, synthetic construct
GGGG

(19) United States
(12) Patent Application Publication
(10) Pub. No.: US 2025/0368701 A1
(43) Pub. Date: Dec. 4, 2025

(54) COILED-COIL PEPTIDES FOR FORCE-DEPENDENT APPLICATIONS

(71) Applicant: Yale University, New Haven, CT (US)

(72) Inventors: Julien Berro, New Haven, CT (US); Yuan Ren, New Haven, CT (US); Yongli Zhang, New Haven, CT (US)

(21) Appl. No.: 18/874,732

(22) PCT Filed: Jun. 30, 2023
(86) PCT No.: PCT/US2023/069505

§ 371 Date: Dec. 13, 2024

Related U.S. Application Data
(60) Provisional application No. 63/357,272, filed on Jun. 30, 2022.

(51) Int. Cl. C07K 14/47; G01N 33/68

(52) U.S. Cl. CPC C07K 14/47; G01N 33/68; C07K 2319/60

(57) ABSTRACT

Force-sensing peptides that can be used as sensor molecules or can be used for signal transduction by detecting a force and translating the force into a biological signal are described as well as methods of using the force-sensing peptides.

### CROSS-REFERENCE TO RELATED APPLICATIONS

This application is entitled to priority under 35 U.S.C. § 119(e) to U.S. Provisional Application No. 63/357,272, filed Jun. 30, 2022, each of which is hereby incorporated by reference in its entirety.

### STATEMENT REGARDING FEDERALLY SPONSORED RESEARCH OR DEVELOPMENT

This invention was made with government support under R21GM132661 and R35GM131714 awarded by the National Institutes of Health. The government has certain rights in the invention.

### BACKGROUND OF THE INVENTION

Mechanical forces are ubiquitous in biological processes. They are central to numerous intra-cellular processes (e.g., cell division, cell motility, organelle morphogenesis, etc.), inter-cellular processes (e.g., adhesion, T-cell antigen recognition, etc.) and developmental processes (e.g., stem cell differentiation, organ development, regeneration, etc.). Despite the ubiquity of forces in biology, there is a lack of generally applicable molecular tools able to detect and quantify forces at the molecular level in vivo. The only currently available tools are difficult to use because they are very large, therefore potentially disruptive, and rely on the measurement of Forster Resonance Energy Transfer (FRET) efficiencies, which are difficult and tedious to perform and control for. In addition, tools that can detect a mechanical signal and use it to trigger a biochemical response do not exist.

Thus, there is a need in the art for tools to detect and utilize mechanical forces in biological processes. This invention satisfies this un-met need.

### SUMMARY OF THE INVENTION

In some embodiments, the invention provides a force sensing peptide comprising two α-helix domains linked by a linker sequence wherein the two α-helix domains form a coiled coil domain, referred to as a closed conformation, in the absence of external force, and further wherein the coiled coil domain undergoes a conformational change to an open conformation in the presence of a force above an uncoiling threshold level for the coiled coil domain.

In some embodiments, the force sensing peptide is operably linked to a target molecule of interest, whereby the force sensing peptide undergoes the conformational change when force is applied to the target molecule of interest.

In some embodiments, the linker sequence comprises at least one functional domain or moiety for generating a signal when the force sensing peptide is in an open conformation. In some embodiments, at least one functional domain or moiety is a fragment of a fluorescent protein, a circularly permuted fluorescent protein, a protein cleavage recognition domain, an RNA binding molecule, a DNA-binding domain, an epitope for recognition by a binding molecule, an ion-binding domain, a lipid-binding domain, a peptide containing residues for post-translational modification, a peptide containing unnatural amino acids, a peptide that is a toxin to cells, a peptide with anti-bacterial activity, a peptide with anti-viral activity, a peptide with anti-fungal activity, a peptide with enzymatic activity, a peptide that modifies the enzymatic activity of other proteins, a peptide containing localization signal for cellular compartments, a peptide containing secretion signal, or a peptide that binds another peptide or protein construct.

In some embodiments, the uncoiling threshold level for the coiled coil domain is at least 3, 4, 5, 6, 7, 8, 9, 10, 11, 12, 13, 14, 15, 16, 17, 18, 19, 20, 21, 22, 23, 24, 25, or more than 25 piconewtons (pN).

In some embodiments, the force sensing peptide comprises at least one α-helix domain of SEQ ID NO:1-SEQ ID NO:40.

In some embodiments, the force sensing peptide comprises at least one set of α-helix domains from:
a) SEQ ID NO:1 and SEQ ID NO:21;
b) SEQ ID NO:2 and SEQ ID NO:21;
c) SEQ ID NO:2 and SEQ ID NO:22;
d) SEQ ID NO:3 and SEQ ID NO:23;
e) SEQ ID NO:4 and SEQ ID NO:23;
f) SEQ ID NO:4 and SEQ ID NO:24;
g) SEQ ID NO:5 and SEQ ID NO:25;
h) SEQ ID NO:6 and SEQ ID NO:26;
i) SEQ ID NO:7 and SEQ ID NO:27;
j) SEQ ID NO:8 and SEQ ID NO:28;
k) SEQ ID NO:9 and SEQ ID NO:29;
l) SEQ ID NO:10 and SEQ ID NO:30;
m) SEQ ID NO:11 and SEQ ID NO:31;
n) SEQ ID NO:12 and SEQ ID NO:32;
o) SEQ ID NO:13 and SEQ ID NO:33;
p) SEQ ID NO:14 and SEQ ID NO:34;
q) SEQ ID NO:15 and SEQ ID NO:35;
r) SEQ ID NO:16 and SEQ ID NO:36;
s) SEQ ID NO:17 and SEQ ID NO:37;
t) SEQ ID NO:18 and SEQ ID NO:38;
u) SEQ ID NO:19 and SEQ ID NO:39; or
v) SEQ ID NO:20 and SEQ ID NO:40,

In some embodiments, the set of α-helix domains are linked by a linker sequence. In some embodiments, the linker sequence is one of SEQ ID NO:41-SEQ ID NO:56.

In some embodiments, the force sensing peptide comprises one of SEQ ID NO:57-SEQ ID NO:80.

In some embodiments, the force sensing peptide is linked to a target molecule of interest. In some embodiments, the target molecule of interest is a detectable moiety, a fluorescent protein, a purification tag, a targeting domain, a cellular localization signal, a DNA molecule, an RNA molecule, cAMP, cGMP, eicosapentaenoic acid, eicosatetraenoic acid, a phosphatidylcholine, a phosphatidylinositol, a phosphatidylethanolamine, an aminomethyl polystyrene linker, a chloromethyl polystyrene linker, a PEG linker, a solid-phase protein synthesis resin, a C-terminal fragment of End4p, a C-terminal fragment of actin, a C-terminal fragment of clathrin, a C-terminal fragment of vinculin, a C-terminal fragment of talin, a C-terminal fragment of integrin, glycogen, cellulose, a therapeutic agent, an antibiotic, an antiviral, an anti-fungal, an anti-helminthic, an anti-inflammatory molecule, or a chemotherapeutic.

In some embodiments, the force sensing peptide is linked to a molecule to tether the force sensing peptide to a surface or substrate.

In some embodiments, the invention provides a system for determination of the level of force exerted on a target of interest, the system comprising at least two force sensing peptides, wherein each of the at least two force sensing peptides have different uncoiling threshold levels.

In some embodiments, the invention provides a nucleic acid molecule encoding a force sensing peptide.

In some embodiments, the invention provides a genetically modified host cell comprising a nucleic acid molecule encoding a force sensing peptide.

In some embodiments, the invention provides a method of detecting the presence or level of force applied to a target molecule of interest, the method comprising contacting a target molecule of interest operably linked to a force sensing peptide with a sufficient level of force to induce a conformational change in the force sensing peptide, and detecting the presence of the open conformation of the force sensing peptide.

In some embodiments, the force sensing peptide comprises a linker sequence comprising at least one functional domain or moiety for generating a signal when the force sensing peptide is in an open conformation.

In some embodiments, at least one functional domain or moiety is a fragment of a fluorescent protein, a circular fluorescent protein, a bioluminescent protein, a protein cleavage domain, an RNA binding molecule, a DNA-binding domain, an epitope for recognition by a binding molecule, an ion-binding domain, a lipid-binding domain, a peptide containing residues for post-translational modification, a peptide containing unnatural amino acids, a peptide that is a toxin to cells, a peptide with anti-bacterial activity, a peptide with anti-viral activity, a peptide with anti-fungal activity, a peptide with enzymatic activity, a peptide that modifies the enzymatic activity of other proteins, a peptide containing localization signal for cellular compartments, or a peptide containing secretion signal.

In some embodiments, the method of detecting the presence or level of force comprises detecting a fluorescent signal that is generated in the presence of the open conformation of the force sensing peptide.

In some embodiments, the method of detecting the presence or level of force comprises detecting a bioluminescent signal that is generated in the presence of the open conformation of the force sensing peptide.

In some embodiments, the method of detecting the presence or level of force comprises detecting a differential level of a protein or an mRNA molecule when the force sensing peptide is in the open conformation.

In some embodiments, the method of detecting the presence or level of force comprises detecting a change in localization of the target molecule due to cleavage of a peptide cleavage domain that is exposed when the force sensing peptide is in the open conformation.

In some embodiments, the force sensing peptide comprises at least one α-helix domain of SEQ ID NO:1-SEQ ID NO:40.

In some embodiments, the force sensing peptide comprises at least one set of α-helix domains from:
a) SEQ ID NO:1 and SEQ ID NO:21;
b) SEQ ID NO:2 and SEQ ID NO:21;
c) SEQ ID NO:2 and SEQ ID NO:22;
d) SEQ ID NO:3 and SEQ ID NO:23;
e) SEQ ID NO:4 and SEQ ID NO:23;
f) SEQ ID NO:4 and SEQ ID NO:24;
g) SEQ ID NO:5 and SEQ ID NO:25;
h) SEQ ID NO:6 and SEQ ID NO:26;
i) SEQ ID NO:7 and SEQ ID NO:27;
j) SEQ ID NO:8 and SEQ ID NO:28;
k) SEQ ID NO:9 and SEQ ID NO:29;
l) SEQ ID NO:10 and SEQ ID NO:30;
m) SEQ ID NO:11 and SEQ ID NO:31;
n) SEQ ID NO:12 and SEQ ID NO:32;
o) SEQ ID NO:13 and SEQ ID NO:33;
p) SEQ ID NO:14 and SEQ ID NO:34;
q) SEQ ID NO:15 and SEQ ID NO:35;
r) SEQ ID NO:16 and SEQ ID NO:36;
s) SEQ ID NO:17 and SEQ ID NO:37;
t) SEQ ID NO:18 and SEQ ID NO:38;
u) SEQ ID NO:19 and SEQ ID NO:39; or
v) SEQ ID NO:20 and SEQ ID NO:40.

In some embodiments, the set of α-helix domains are linked by a linker sequence. In some embodiments, the linker sequence is one of SEQ ID NO:41-SEQ ID NO:56. In some embodiments, the force sensing peptide comprises one of SEQ ID NO:57-SEQ ID NO:80.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1H, depicts schematics and representative data from insertion of a calibrated coiled-coil force sensor within End4p. FIG. 1A depicts a schematic diagram of End4p linking the lipid membrane to F-actin at sites of clathrin-mediated endocytosis. Upper left, cross section of a fission yeast cell with endocytic pits at different stages. Lower left, side view of an endocytic pit with invaginated membrane. Right, End4p dimers traverses the clathrin coat (gray dotted curve) to transmit the forces generated by the assembly of actin filaments (magenta rods) to deform the plasma membrane. Cyan: End4p lipid binding AP180 N-Terminal Homology (ANTH) domain; yellow: End4p proline rich domain; magenta: End4p F-actin binding talin-HIP1/R/Sla2p actin-tethering C-terminal homology (THATCH) domain. Drawings are not to scale. FIG. 1B depicts a schematic representation of an End4p dimer with the insertion of the coiled-coil force sensor before the THATCH domain. FIG. 1C depicts a schematic of the constructs used to calibrate the coiled-coil force sensor with optical tweezers. The coiled-coil is attached to two beads trapped by optical tweezers through a 2260-bp DNA handle. FIG. 1D depicts representative force-extension curves obtained by pulling (back curve) and then relaxing (red curve) the coiled-coil force sensor. FIG. 1E depicts a representative distribution of the unfolding force for the coiled-coil force sensor cc-11pN (N=46). The average unfolding force is presented as mean±SEM. FIG. 1F depicts representative fission yeast cells expressing fluorescently-tagged wild-type End4p. End4p is mostly found in endocytic patches that are enriched at cell tips and the division plane. See also FIG. 2. FIG. 1G depicts representative fission yeast cells expressing fluorescently-tagged End4p with cc-11pN inserted before the THATCH domain as in FIG. 1B. Multiple large persistent spherical condensates of End4p constructs (indicated by arrows) can be distinguished from transient diffraction limited endocytic patches. FIG. 1H depicts representative imaging of End4p condensates displaying liquid-like behaviors. Upper panel, fusion of two End4p condensates (arrow). Lower panel, fission of one End4p condensate into two smaller condensates (arrow). See the full dynamics of End4p condensates in FIG. 3. FIGS. 1F and 1G, scale bars: 5 μm. FIG. 1H, scale bars: 2 μm. The schematic under each image indicates the modifications on End4p, and fluorescent tags are omitted for clarity.

FIG. 2, comprising FIGS. 2A and 2B, depicts a schematic of End4p. FIG. 2A depicts a schematic of End4p dimer. The numbers indicate the residues where domains start and end. The coiled-coil force sensors are inserted after D155, P337, or V856. Drawings are not to scale. FIG. 2B depicts a schematic of the beginning of End4p dimerization coiled-coil, which contains a poly-Q region that is predicted to be prion like by Prion Like Amino Acid Composition (PLAAC) (Lancaster et al., 2014, Bioinformatics, September 1; 30(17):2501-2502) Glutamines (Q) are highlighted in red.

FIG. 3, comprising FIG. 3A through FIG. 3C, depicts representative data demonstrating that End4p constructs in condensates have a longer lifetime than End4p in endocytic sites. FIG. 3A depicts representative fission yeast cells with fluorescently tagged wild-type End4p. Right: temporal evolution of End4p in the boxed region. A typical End4p patch lasts less than 50 seconds. FIGS. 3B and 3C depict representative fission yeast cells with fluorescently-tagged End4p where a cc-11pN was inserted before the THATCH domain. Right: temporal evolution of End4p construct in the boxed regions. End4p condensates experience rapid movements across the cell and occasionally fuse with each other (FIG. 3B) or split (FIG. 3C) (arrows). A subset of this montage is shown in FIG. 1G. Scale bars in the left panels, 5 μm. Scale bars in the right panels, 2 μm. End4p is tagged at the C-terminal with mEGFP. Images are inverted contrast of max projected whole cell sections. Note that the contrast is enhanced in FIG. 3A to better visualize a single endocytic event.

FIG. 4, comprising FIG. 4A through FIG. 4F, depicts schematic diagrams of coiled-coil force sensors in different conformations. FIG. 4A depicts a ribbon diagram of cc-11pN in the folded state. The two α-helices (blue and magenta) are connected with a 30-amino acid linker (black line). Dimensions of the coiled-coil are indicated according to the structure of PDB entry 2zta. FIG. 4B depicts the structure of a folded cc-11pN with the residues in the hydrophobic core highlighted. These hydrophobic residues will be exposed when the coiled-coil unfolds under sufficiently large force. FIG. 4C depicts cc-11pN with a 30-amino acid linker folds properly by forming a parallel coiled-coil. FIG. 4D depicts cc-11pN with a 10-amino acid is unable to form a parallel coiled-coil and adopts an open conformation. This open conformation promotes the formation of protein condensates as shown in FIG. 5A-5B. FIG. 4E depicts an intermolecular connection between two open cc-11pN is formed during the refolding process. Each intermolecular connection generates two exposed α-helices that could mediate further intermolecular connections. FIG. 4F depicts cleavage at the linker of the two α-helices of cc-11pN prevents the formation of intermolecular connections. See also FIG. 5F. The N- and C-terminals of α-helices in FIG. 4A-4F are labeled for orientation.

FIG. 5, comprising FIG. 5A through FIG. 5K, depicts force-induced unfolding of the coiled-coil force sensor promoting the formation of End4p condensates. FIG. 5A depicts a schematic of End4p condensate formation. The coiled-coil force sensor unfolds when the magnitude of force on End4p exceeds the unfolding force threshold of the coiled-coil force sensor. During the refolding process, α-helices from different End4p molecules mediate the entanglement of End4p molecules into condensates. See also FIG. 4E. FIG. 5B-5I depict representative localization of different End4p constructs in fission yeast cells. Both the N- and C-terminal End4p fragments are fluorescently labeled in FIG. 5E-5F. FIG. 5B depicts the insertion of an open conformation cc-11pN into End4p led to larger End4p condensates. See also FIG. 4C-D, FIG. 6. FIGS. 5C-5E depict the insertion of cc-11pN into End4p does not lead to condensate formation at the absence of force. The force on cc-11pN was removed by inserting after the THATCH domain (FIG. 5C), deleting the THATCH domain (FIG. 5D), or disconnecting the THATCH domain with the rest of End4p (FIG. 5E). FIG. 5F depicts the formation of End4p condensates is dependent on the connection between the two α-helices of cc-11pN. The cleaving of the linker of cc-11pN prevented the formation of condensates. See also FIG. 4E-F. FIG. 5G-5H depict representative data demonstrating that End4p does not form condensates when a cc-11pN was inserted before the THATCH domain, and RVK1010DDD (FIG. 5G) or R1093G (FIG. 5H) mutations were introduced into the THATCH domain to abolish its binding to F-actin. FIG. 5I depicts representative snapshots of fission yeast cells with End4p condensates before and after the treatment with 100 μM LatA. The size (FIG. 5J) and number (FIG. 5K) of End4p condensates decreases after actin assembly is impaired by the latA treatment. Data in 5J-5K are presented as the mean±SD (>300 condensates from n=3 independent repeats). Asterisk indicates a significant difference. FIG. 5J, P=0.0003, two-tailed t test. FIG. 5K, P=0.01, two-tailed t test. FIG. 5B-5H, Scale bar in FIG. 5B applies to all images, 5 μm. End4p is tagged at the C-terminal with mEGFP in FIG. 5B-5I, and End4p is also tagged at the N-terminal with mScarlet-I in FIG. 5E-5F. In vivo protein cleaving in FIG. 5E-5F was achieved by the insertion of a 2A peptide. The schematic under each image indicates the modifications on End4p, and fluorescent tags are omitted for clarity. See the localization of End4p without cc-11pN in FIG. 7.

FIG. 6, comprising FIG. 6A through 6I, depicts representative quantification of protein condensates. End4p condensates were differentiated from End4p patches by a custom ImageJ plugin and analyzed for comparison of sizes. FIGS. 6A-6C depict quantification when End4p was tagged at the N-terminal with mEGFP. FIGS. 6D-6F depict quantification when End4p was tagged at the N-terminal with mEGFP, and a cc-11pN was inserted before the THATCH domain. FIGS. 6G-6I depict quantification when End4p was tagged at the N-terminal with mEGFP, and a cc-11pN in the open conformation was inserted before the THATCH domain. FIGS. 6B, 6E, and 6H depict representative images of fission yeast cells with fluorescently tagged End4p or End4p constructs are shown on the left, and the identified End4p condensates are shown on the right. The distributions of condensate diameter are shown in FIGS. 6C, 6F, and 6I, each from >500 cells. A negligible population of End4p patches, typically from spatially overlapping endocytic events, is misidentified as End4p condensates in FIG. 6B-6C. Scale bar in FIG. 6B applies to all images, 10 μm. Images in FIGS. 6B, 6E, and 6H were acquired and displayed with the same settings.

FIG. 7, comprising FIG. 7A through FIG. 7E, depicts representative data for controls for End4p localization in mutant strains. FIG. 7A depicts representative fission yeast cells expressing End4p where a self-cleaving 2A peptide was inserted before the THATCH domain and fluorescently-tagged at its N- and C-terminals. The End4p N-terminal abnormally accumulates at cell tips and the division plane, while End4p C-terminal is diffusive in the cytoplasm. The localization of End4p fragments were not changed by the insertion of cc-11pN as shown in FIG. 5E. FIG. 7B depicts representative fission yeast cells expressing fluorescently-tagged End4p where the THATCH domain was deleted. End4p without the THATCH domain abnormally accumulates at cell tips and the division plane. The localization of this End4p construct was not changed by the insertion of cc-11pN as shown in FIG. 5D. FIG. 7C depicts representative fission yeast cells expressing fluorescently-tagged End4p where a self-cleaving 2A peptide was inserted after the proline rich domain. End4p N-terminal is diffusive in the cytoplasm, while its C-terminal localizes to endocytic patches. Compare with FIG. 9B. FIG. 7D depicts representative fission yeast cells expressing fluorescently-tagged End4p where the RVK1010DDD mutation was introduced into the THATCH domain to abolish binding to actin filaments. This mutant abnormally accumulates at cell tips and the division plane. FIG. 7E depicts representative fission yeast cells expressing fluorescently-tagged End4p where the R1093G mutation was introduced into the THATCH domain to abolish binding to actin filaments. This mutant abnormally accumulates at cell tips and the division plane. The localization of End4p in FIG. 7D-7E were not changed by the insertion of cc-11pN as shown in FIGS. 5G and 5H. FIG. 7A-7E, Scale bar in FIG. 7A applies to all images, 5 μm. Images are inverted contrast of max projected whole cell sections. End4p is tagged at the N-terminal with mScarlet-I in FIGS. 7A-7E, and End4p is also tagged at the C-terminal with mEGFP in FIGS. 7A and 7C-7E. The schematic under each image indicates the modifications on End4p, and fluorescent tags are omitted for clarity.

FIG. 8, comprising FIG. 8A through FIG. 8B, depicts representative data demonstrating that forces in End4p can be measured by inserting calibrated coiled-coils between different domains. FIG. 8A depicts End4p localization when a calibrated coiled-coil force sensor that unfolds at 14pN (cc-14pN) was inserted before the Rend domain. The formation of End4p condensates indicate that force before Rend is above 14pN. FIG. 8B depicts End4p localization when a calibrated coiled-coil force sensor that unfolds at 18pN (cc-18pN) was inserted before the THATCH domain. The formation of End4p condensates indicate that force before THATCH is above 18pN.

FIG. 9, comprising FIG. 9A through FIG. 9E, depicts representative data demonstrating that End4p is under a force gradient. FIG. 9A depicts End4p localization when a calibrated coiled-coil force sensor was inserted after the ANTH domain (first column), after the proline rich domain (second column), or before the THATCH domain (third column). Coiled-coils unfolding thresholds are 20 pN (first row), 11 pN (second row), 10 pN (third row), and 8 pN (fourth row). Images containing End4p condensates are boxed in blue frames. FIG. 9B depicts End4p N- and C-terminal localization when a cc-11pN was inserted before the THATCH domain, and a self-cleaving 2A peptide was introduced after the proline rich domain. The End4p C-terminal fragment formed condensates despite its disconnection from the lipid-binding ANTH domain, demonstrating that at least 11 pN of force is transmitted by End4p C-terminal (after the proline-rich domain) to other components of the endocytic machinery. See the localization of End4p fragments without cc-11pN in FIG. 7C. FIG. 9C depicts End4p localization when a cc-8pN was inserted after the ANTH domain, and an R1093G mutation was introduced into the THATCH domain to abolish its binding to actin filaments. This End4p construct formed condensates despite its inability to bind actin, demonstrating that at least 8 pN of force is transmitted by other components of the endocytic machinery to End4p ANTH domain. FIG. 9D depicts End4p localization when a cc-8pN was inserted after the proline rich domain, and an R1093G mutation was introduced into the THATCH domain to abolish its binding to F-actin. This construct didn't form condensates. This result and the result of panel FIG. 9C demonstrate that most of the force not transmitted through the THATCH domain is transmitted through the proline rich domain. FIG. 9E depicts a schematic model of force transmission during CME. Top: distribution of forces on End4p. The new coiled-coil library allowed the determination that the forces between different domain of End4p are different. Bottom: hypothetical organization of adaptor proteins across the clathrin coat. End4p and Ent1p make direct interactions with the plasma membrane, the clathrin lattice and actin filaments, and are known mechanical linkers to transmit the forces produced by the actin meshwork to deform the membrane. Other endocytic proteins or protein complexes, through their binding to domains in End4p or Ent1p, also relay force from F-actin to the membrane. Double arrows indicate known protein-protein interactions. Drawings are not to scale. Scale bar in FIG. 9A applies to all images, 5 μm. End4p is tagged at the C-terminal with mEGFP in FIG. 9A-9D, and End4p is also tagged at the N-terminal with mScarlet-I in FIG. 9B. The schematic under each image indicates the modifications on End4p, and fluorescent tags are omitted for clarity.

FIG. 10, comprising FIG. 10A through FIG. 10D, depicts temporal evolution of End4p patches at endocytic sites. FIGS. 10A-10D depict the temporal evolution of End4p with or without coiled-coil force sensors is recorded at endocytic sites. The insertion of a coiled-coil force sensor with an unfolding force threshold higher than the local force does not affect the assembly and disassembly of End4p molecules. Scale bar, 2 μm. End4p is tagged at the C-terminal with mEGFP in FIGS. 10A-10D. Images are inverted contrast of max projected whole cell sections. The schematic above each image indicates the location of coiled-coil force sensors in End4p, and fluorescent tags are omitted for clarity.

FIG. 11 depicts a representative comparison of End4p protein expression levels in different strains. The protein expression level of End4p was quantified by summing the whole cell fluorescence of C-terminally tagged End4p with mEGFP after background subtraction. WT: no insertion of coiled-coil force sensor (representative image in FIG. 1F). cc-11pN: 11pN coiled-coil force sensor inserted before THATCH (representative image in FIG. 1G). cc-11pN open: open conformation cc-11pN inserted before THATCH (representative image in FIG. 5B). Dunn's multiple comparison. ****: P<0.0001. Each dot represents one field of view. >200 cells for each group from at least three independent repeats

FIG. 12 depicts representative imaging demonstrating force transmission to the membrane is robust against perturbation. A mutant background was created by removing the actin cytoskeleton-binding (ACB) domain of Ent1p, and calibrated coiled-coil force sensors were inserted after the ANTH domain (first column), after the proline rich domain (second column), or before the THATCH domain (third column). The force before the THATCH domain increased to more than 20pN, whereas forces after the ANTH domain or after the proline rich domain remained unchanged. Scale bar, 5 μm. The image containing End4p condensates are boxed in blue frames. End4p is tagged at the C-terminal with mEGFP, and the fluorescent tags in the schematics are omitted for clarity.

FIG. 13, comprising FIG. 13A through FIG. 13E, depicts a representative library of calibrated coiled-coil force sensors. FIGS. 13A-13D depict schematic sequence and the pulling direction (indicated by black arrows) of coiled-coil force sensors. Among the four force sensors, cc-8pN, cc-11pN and cc-20pN (FIG. 13A, FIG. 13C, and FIG. 13D, respectively) are parallel coiled-coils while cc-10pN (FIG. 13B) is an anti-parallel coiled-coil. The flexible linker between the two α-helices (blue and magenta) are drawn as black lines. FIG. 13E depicts representative average unfolding forces of the coiled-coil force sensors, presented as mean±SEM. N=29 for cc-8pN. N=45 for cc-10pN. N=48 for cc-20pN. See the distribution of unfolding forces and controls of the force sensors in FIG. 14.

FIG. 14, comprising FIG. 14A through FIG. 14G, depicts representative average unfolding forces of coiled coils. FIGS. 14A-14C depict representative distribution of the unfolding force for cc-8pN, cc-10pN and cc-20pN. Data are used to calculate the average unfolding force as shown in FIG. 13E. FIGS. 14D-14F depict representative fission yeast cells expressing fluorescently-tagged End4p where cc-8 pN, cc-10 pN or cc-20 pN was inserted before the THATCH domain, and an R1093G mutation was introduced into the THATCH domain to abolish its binding to actin filaments. End4p shows abnormal accumulation at cell tips and the division plane, without forming condensates. FIG. 14G depicts representative fission yeast cells expressing fluorescently-tagged End4p where a variant of cc-20pN that is always in an open conformation was inserted before the THATCH domain. The insertion of this “always-open” coiled-coil into End4p led to End4p condensates. FIGS. 14D-14G: scale bar in FIG. 14D applies to all images, 5 μm. Images are inverted contrast of max projected whole cell sections. The schematic under each image indicates the modifications on End4p, and fluorescent tags are omitted for clarity.

FIG. 15 depicts a representative summary of a library of coiled-coil designs and the corresponding force threshold. Five families of templates are depicted (Dynein stalk, Peptide Oakley, Peptide Velcro, VBP coiled-coil, and GCN4 coiled-coil). The predicted force ranges of each family are indicated in a brackets. The number of variants in the representative library corresponding to each family is in parentheses. The red circles indicate coiled-coils within the representative library that have been calibrated in vitro using optical tweezers, and are located at positions corresponding to their mean unfolding force threshold.

FIG. 16, comprising FIG. 16A through FIG. 16D, depicts schematic and representative force sensors with fluorescent reporters. FIG. 16A depicts a schematic representation of how a GFP11 peptide can be introduced in the loop between both α-helices of the coiled-coil. GFP1-10 is expressed in the cytoplasm and is nonfluorescent when it is not bound to GFP11. When force is low, the coiled-coil remains closed and the GFP11 peptide is not accessible. When the force is high, the coiled-coil opens and exposes GFP11 which can bind to GFP1-10, reconstituting a fluorescent GFP. FIG. 16B depicts a coiled-coil force sensor combined with a cpGFP. Under low force the coiled-coil is folded; under high force the coiled-coil unfolds and the structural change disrupts the cpGFP fluorescence. FIG. 16C depicts representative data showing the split-GFP strategy in vivo. GFP11 is inserted in the loop of a sensor that opens at 3pN, and forces on End4p are probed. When the sensor is not under force, fluorescence cannot be detected. When force is present, fluorescence is detected and shows the localization of the subset of End4p that is under force within the cell. FIG. 16D depicts fluorescent force sensors characterized for the change in the fluorescence ratio when excited at 400 and 490 nm (change in 400/490 ratio, left) or of their peak fluorescence (ΔF/Fmin ratio, right) in the folded (black) and unfolded states (red). These changes can be used to identify whether the force applied on the protein construct in cells is larger than the force sensor's threshold.

FIG. 17, comprising FIG. 17A through FIG. 17G, depicts representative methods of detecting unfolded coiled-coils. FIG. 17A depicts a representative image of GFP fluorescence in dividing yeast cells expressing a 3 pN coiled-coil-GFP₁₁ sensor inserted into Ent1p, showing fluorescence after GFP reassembly, indicating the peak force on Ent1p is at least 3 pN. FIG. 17B depicts a representative image of GFP fluorescence in dividing yeast cells expressing a 5 pN coiled-coil-GFP₁₁ sensor inserted into Ent1p, showing fluorescence after GFP reassembly, indicating the peak force on Ent1p is less than 5 pN. FIG. 17C depicts a representative image of GFP fluorescence in dividing yeast cells expressing a 7 pN coiled-coil-GFP₁₁ sensor inserted into Ent1p, showing fluorescence after GFP reassembly, verifying the peak force on Ent1p is less than 7 pN. FIG. 17D depicts a schematic representation of a coiled-coil sensor with a binder linker, which unfolds upon application of force, causing real-time enrichment of fluorescence. FIG. 17E depicts a representative kymograph of a 3 pN coiled-coil-IAAL-K3 peptide sensor inserted into Ent1p, with images taken every two seconds. GFP-IAAL-E3 is recruited to the endocytic site only when the force on Ent1p exceeds 3 pN. FIG. 17F depicts a schematic representation of coiled-coil sensor with a HiBiT linker, which is inaccessible until adequate force is applied, at which point LgBiT and the exposed HiBiT form enzymatically active NanoLuc. FIG. 17G depicts fluorescence measurements of dividing yeast cells expressing a 3 pN coiled-coil-HiBiT linker inserted into Ent1p. 1: negative control, no HiBiT; 2-4: Ent1p variants with a 3 pN coiled-coil HiBiT linker inserted; 5: positive control, HiBiT attached to the C-terminus of Ent1p. For FIG. 17A through FIG. 17C, scale bar=5 μm.

FIG. 18, comprising FIG. 18A through FIG. 18B, depicts schematic representations of focal adhesion proteins. FIG. 18A depicts a schematic of focal adhesion components in mammalian cells. FIG. 18B depicts Talin and Vinculin constructs with force sensors in place of previously reported FRET-based tension-sensing modules (TS module) for comparison of measurements by each method.

FIG. 19 depicts a schematic diagram of a strategy for identifying proteins that modulate force in a subcellular process. Once the force on a protein of interest (POI) is known, mutations on other proteins can describe their role in force production. In this diagram, the force on the POI is 7 pN, as demonstrated using split-GFP sensors; the POI is visible (green dots) only if the force exerted on the POI is larger than the threshold force of the sensor. If, for a given force sensor, the POI is not visible anymore when protein X is mutated, the force decreased and that protein enhances the force production or transmission to the POI in wild-type.

FIG. 20 depicts a schematic for a method of identifying locations under force within a protein of interest. By inserting a weak force sensor (e.g., 3 pN unfolding threshold) at different sites between domains of a protein of interest and observing which insertions are unfolded, force-bearing domains are identified. Insertion of the always closed variant of this coiled-coil (or a coiled-coil with very large unfolding force) will control that the observed phenotype is not an artifact.

FIG. 21 depicts a schematic for a strategy to determine the force on a protein of interest (POI) using a library of force sensors. By testing a range of force sensors with gradually increasing unfolding thresholds, the force on the POI can be determined by dichotomy, i.e. the force is larger than the threshold forces of sensors that allows a positive readout (here reconstructed fluorescence of the split-GFP), and smaller than the threshold forces of sensors that keep the protein construct not fluorescent (between 6 and 8 pN in this cartoon).

FIG. 22, comprising FIG. 22A through FIG. 22D, depicts representative results of coiled-coil sensor insertion into a C. elegans protein. FIG. 22A depicts a representative DIC image of a nematode with the position of the sensory PLM neuron (solid line) illustrated in a DIC image of a nematode. Coordinates on the left show the anterior-posterior (A, P) and ventral-dorsal (V,D) axis. The PLM cell body (*) is located close to the tail of the nematode, where it extends a short neurite posterior towards the tail and a long neurite anterior towards the center of the animal. Along this anterior neurite, PLM forms a short branch to innervate interneurons in the ventral nerve cord. The dashed line along the entire length of the neurites indicates the analyzed region for FIG. 22D. Scale bar=100 μm. FIG. 22B depicts schematic representations of the membrane-associated periodic skeleton (MPS, left) and UNC-70 constructs (Right). The left schematic representation demonstrates spectrins forming the central building block of the membrane associated periodic skeleton (MPS), which consists of actin rings that are interspaced by spectrin tetramers and form a periodic lattice below the plasma membrane throughout the entire length of the axon. Each spectrin tetramer spaces a length of approximately 200 nm and consists of two heterodimers of α(SPC-1 in nematodes)- and β-spectrin (UNC-70 in nematodes) subunits, that assemble head-to-head. The right schematic representation depicts the domain organization in UNC-70/0-spectrin with location of the force sensor probe insertion. A force sensor or GFP11 repeat (control) were inserted into the genomic unc-70 locus at the position that encodes for the linker region between spectrin repeats 8 and 9. CH: Calponin Homology Domain, SR: Spectrin-like Repeats, PH: Pleckstrin Homology Domain. FIG. 22C depicts representative confocal fluorescence images of animals expressing endogenous force-sensor-GFP₁₁ or GFP₁₁ (control) tagged UNC-70 together with GFP1-10 under a PLM specific promoter (mec-17p) in a wildtype or unc-115(ky275) mutant background. Unc-115(ky275) leads to a loss of the periodic MPS lattice in PLM. Signal intensity is color coded according to the LUT. Scale bar=50 μm. The position of the cell body (*) and the rectum (#) are indicated. Note that the rectum as well bright speckles in the animal's body represent background fluorescence. FIG. 22D depicts quantification of fluorescence depicted in FIG. 22C. Indicated p-values are derived from a statistical comparison based on a Kruskal Wallis test followed by Dunn's test for multiple comparisons. nGFP₁₁=11, n_{3pN Sensor}=12, n_{10pN Sensor}=17, n_{GFP11; unc-115(ky275)}=11, and n_{3pN; unc-115(ky275)}=11 animals.

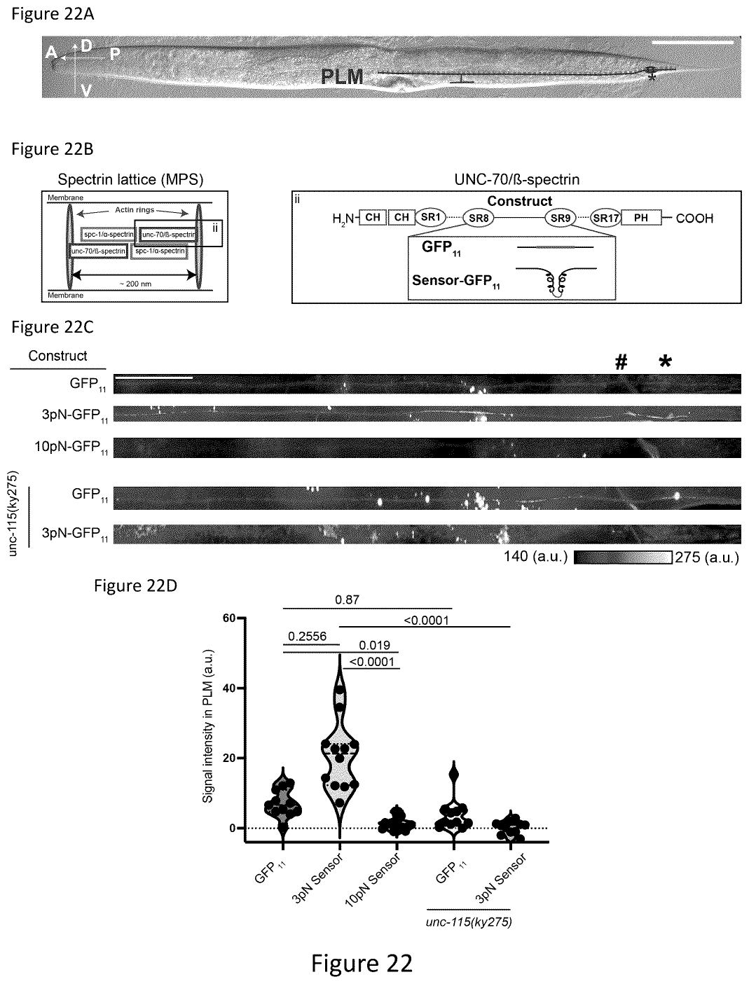

### DETAILED DESCRIPTION OF THE INVENTION

The present invention relates to compositions and methods for utilizing force-dependent protein activity. In certain aspects, the compositions and methods of the present invention relate to force sensor constructs. The force sensors can be used in vivo or in vitro. In some embodiments, the sensor construct can be expressed or inserted into a cell. The cell can be of any organism that can express the sensor construct. In some embodiments, the composition comprises a coiled coil domain comprising a first coil element, a linker domain, and a second coil element. In certain embodiments a first protein domain is conjugated to the first coil element and a second protein domain is conjugated to the second coil element. In some embodiments, composition comprises a protein wherein the first coil element, linker domain, and second coil element are inserted into the amino acid sequence of a protein of interest at a location that is innocuous to function and localization of the protein.

In some embodiments, the composition comprises a linker domain comprising a functional domain or moiety. In some embodiments, the linker comprises a peptide that induces condensation of the composition. In some embodiments, the linker comprises a peptide that binds to another peptide or protein domain directly or indirectly. In some embodiments, the linker comprises an epitope for recognition by a binding molecule. In some embodiments, the linker comprises a domain for interaction with a small molecule or intermediate of a small molecule. In some embodiments, the linker comprises a GFP11 peptide. In some embodiments, the linker comprises a circularly permutated fluorescent protein. In some embodiments, the linker comprises an RNA-binding domain. In some embodiments, the linker comprises an IAAL-K3/IAAL-E3 binding peptide. In some embodiments, the linker comprises a fragment of luciferase.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “activate,” as used herein, means to induce or increase a molecule, a reaction, an interaction, a gene, an mRNA, and/or a protein's expression, stability, function or activity by a measurable amount relative to a control comparator. “Activators” are compounds that, e.g., bind to, partially or totally induce stimulation, increase, promote, induce activation, activate, sensitize, or up regulate a protein, a gene, and an mRNA stability, expression, function and activity, e.g., agonists.

The term “assessing” includes any form of measurement, and also includes determining if an element is present or not. The terms “determining,” “measuring,” “evaluating,” “assessing” and “assaying” are used interchangeably and may include quantitative and/or qualitative determinations. Assessing may be relative or absolute. “Assessing binding” includes determining the amount of binding, and/or determining whether binding has occurred (i.e., whether binding is present or absent). “Assessing activity” includes determining the amount of activity, and/or determining whether an activity has occurred (i.e., whether an activity is present or absent).

As used herein, the term “bind” or “binding” refers to the specific association or other specific interaction between two molecular species, such as, but not limited to, protein-DNA/RNA interactions and protein-protein interactions, for example, the specific association between proteins and their DNA/RNA targets, receptors and their ligands, enzymes and their substrates, etc. Such binding may be specific or non-specific, and can involve various noncovalent interactions, such as including hydrogen bonding, metal coordination, hydrophobic forces, van der Waals forces, pi-pi interactions, and/or electrostatic effects.

As used herein, the term “coil” refers to a peptide α-helix structure, or a structure similar in nature or appearance to a peptide α-helix. As used herein, the term “coil” is not limited to the 3.6₁₃-helix.

As used herein, the term “coiled-coil” refers to structural protein motifs comprising 2 or more α-helices that twist around each other forming a super coil. They generally contain a heptad repeat designated (a-b-c-d-e-f-g)ₙ every two turns of a helix, “a” and “d” usually represent nonpolar, hydrophobic residues that are found at the interface of the two helices, “e” and “g” are solvent exposed polar residues that interact electrostatically, “b”, “c” and “f” are hydrophilic and exposed to the solvent. Different amino acids on positions “a-g” define oligomerization state, specify, helix orientation and stability. The term “coiled coil” as used herein can also refer to supercoils that are made from non-3.613-helices and have repeating units different than the (a-b-c-d-e-f-g)ₙ heptad. The term coiled-coil domain in the description refers to naturally occurring or designed coiled-coil protein structure motifs which comprise at least two heptads and can be parallel or antiparallel.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the polynucleotides' bases are matched according to the base pairing rules. Or, there may be “complete” or “total” complementarity between the polynucleotides. The degree of complementarity between polynucleotide strands has significant effects on the efficiency and strength of hybridization between polynucleotide strands. This is of particular importance in amplification reactions, as well as detection methods that depend upon binding between polynucleotides.

“Contacting” refers to a process in which two or more molecules or two or more components of the same molecule or different molecules are brought into physical proximity such that they are able to undergo an interaction. Molecules or components thereof may be contacted by combining two or more different components containing molecules, for example by mixing two or more solution components, preparing a solution comprising two or more molecules such as target, candidate or competitive binding reference molecules, and/or combining two or more flowing components. Alternatively, molecules or components thereof may be contacted combining a fluid component with molecules immobilized on or in a cell or on or in a substrate, such as a polymer bead, a membrane, a polymeric glass substrate or substrate surface derivatized to provide immobilization of target molecules, candidate molecules, competitive binding reference molecules or any combination of these. Molecules or components thereof may be contacted by selectively adjusting solution conditions such as, the composition of the solution, ion strength, pH or temperature. Molecules or components thereof may be contacted in a static vessel, such as a microwell of a microarray system, or a flow-through system, such as a microfluidic or nanofluidic system. Molecules or components thereof may be contacted in or on a variety of cells, media, liquids, solutions, colloids, suspensions, emulsions, gels, solids, membrane surfaces, glass surfaces, polymer surfaces, vesicle samples, bilayer samples, micelle samples and other types of cellular models or any combination of these.

As used herein, the terms “downstream” or “upstream” with respect to a signaling pathway is based on epistatic relationships in a linear signaling cascade: if “A” activates “B” and “B” activates “C”, the “A” is upstream of “B” and “B” is upstream of “C”.

Similarly, “B” is downstream of “A” and “C” is downstream of “B”. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

A “fragment” of a polynucleotide sequence that encodes an antigen may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encodes a polypeptide that is 95% or more, 96% or more, 97% or more, 98% or more or 99% or more identical to the polypeptide and additionally optionally comprise sequence encoding an N terminal methionine or heterologous signal peptide which is not included when calculating percent identity.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Identical” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and mRNA stability, expression, function and activity, e.g., antagonists.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “modulate” or “modulator”, as used herein, refers to the activation or inhibition of molecule, as described in the respective definitions herein.

The term “overexpressed” or “overexpression” is intended to indicate an abnormal level of expression of a particular gene in a cell from a disease area like a solid tumor within a specific tissue or organ of the patient relative to the level of expression in a normal cell from that tissue or organ. Patients having solid tumors or a hematological malignancy characterized by overexpression of a particular gene can be determined by standard assays known in the art.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including, but not limited to deletion, insertion, substitution of one or more nucleotides, or fusion to other polynucleotide sequences.

A “variant” of a peptide according to the present invention may be (i) one in which one or more of the amino acid residues are substituted with a conserved or non-conserved amino acid residue and such substituted amino acid residue may or may not be encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptide, and/or (v) one in which the peptide is fused with another peptides, such as a leader or secretory sequence or a sequence which is employed for purification (for example, His-tag) or for detection (for example, Sv5 epitope tag or immunoglobulin Fc region). The fragments include peptides generated via proteolytic cleavage (including multi-site proteolysis) or an original sequence. Variants may be post-translationally, or chemically modified. Such variants are deemed to be within the scope of those skilled in the art from the teaching herein.

A “variant” of a coiled-coil can include peptides with an extension of a coiled-coil, with extra heptads from the same coiled-coil, with extra heptads from other coiled-coils, modifications of residues, including replacing, adding, or deleting residues, and chemical modifications on residues, within the coiled-coil, or any combination thereof, so long as the variant is able to form a coiled-coil structure.

A “vector” is a composition of matter which comprises an isolated polynucleotide and which can be used to deliver the isolated polynucleotide to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of polynucleotide into cells, such as, for example, polylysine compounds, liposomes, and the like. Examples of viral vectors include, but are not limited to, adenoviral vectors, adeno-associated virus vectors, retroviral vectors, and the like.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention is based, in part, on the development of force-sensing peptides. In certain embodiments, the force-sensing peptides can be used as sensor molecules or can be used for signal transduction by detecting a force and translating the force into a chemical, physical, or biological signal. The force-sensing peptides are based on unfolding of structured peptides under force. The peptide unfolding exposes residues that are inaccessible when the peptide is folded. These exposed residues of the sensors can be tuned to react with other molecules, which can be used for detection or actuation.

In some embodiments, the present invention relates to compositions for utilizing forces acting upon proteins. In some embodiments, the composition measures the force acting upon a protein. In some embodiments, the composition detects protein interactions. In some embodiments, the composition makes a domain accessible upon application of a sufficient force.

In some embodiments, the force sensing peptide of the invention comprises a domain comprising a structure that can undergo a change in response to force. In some embodiments, the structure is a three-dimensional fold. In some embodiments, the structure comprises a folded domain that becomes un-folded or opened in response to force. Exemplary structures that can be incorporated into the force sensor peptides of the invention include, but are not limited to, coiled coils, chimeric SNARE complexes, R domains for talins, collagen fibers, spectrin repeats, amyloids, glycoproteins, or other structures that can undergo a conformational change in response to biologically relevant levels of force (e.g., forces in the range of 0.1-100 piconewtons).

In various embodiments, the force sensing peptide of the invention comprises at least one coiled-coil comprising at least two α-helix domains, or coil elements, linked by a flexible linker which allows them to wrap around each other. In some embodiments, the force sensing peptide of the invention comprises two, three, four or more than four α-helix domains which wrap around each other to form a coiled coil with two, three, four or more than four strands.

In some embodiments, a first α-helix domain or coil element is conjugated to a first protein domain. In some embodiments, a second α-helix domain or coil element is conjugated to a second protein domain. In some embodiments, the composition comprises one or more peptide linker between two or more α-helix domains. In some embodiments, the peptide linker is conjugated to a first coil element at its C-terminus. In some embodiments, the peptide linker is conjugated to a second coil element at its N-terminus. In some embodiments, the peptide linker comprises a functional peptide.

In some embodiments, the application of force to the coiled-coil sensor peptide of the invention induces a conformational change of the coiled coil domain, in which the two α-helix domains become separated from each other (referred to herein as the open conformation) exposing amino acid residues for interaction that were protected in the coiled-coil structure (referred to herein as the closed conformation). In some embodiments, the open conformation of the sensor peptides of the invention forms aggregates with one or more additional sensor peptide in the open conformation.

In some embodiments, the force sensor peptide comprises a folded domain that undergoes multiple changes (e.g., multi-step unfolding). In such an embodiment, a first change or unfolding may occur at a first level or occurrence of force and a second change or unfolding may occur upon encountering a second level or occurrence of force. For example, a coiled coil domain comprising three or four coiled strands may undergo a first uncoiling of a first strand when the sensor is contacted with a first instance or first level of force and a second uncoiling of a second strand when the sensor is contacted with a second instance or second level of force. In such an embodiment, the first uncoiling and second or more uncoiling events may be detected individually by different functional domains on linkers that are exposed upon each uncoiling event.

In some embodiments, the force required for inducing a conformational change in the force sensor peptide from the closed to open conformation is at least 3 piconewtons (pN). In some embodiments, the force required for inducing a conformational change in the force sensor peptide from the closed to open conformation is 3, 4, 5, 6, 7, 8, 9, 10, 11, 12, 13, 14, 15, 16, 17, 18, 19, 20, 21, 22, 23, 24, 25 or more than 25 piconewtons (pN).

In some embodiments, the invention provides a series of force sensor peptides which undergo a conformational change from a closed to open conformation in the presence of different levels of force. For example, in some embodiments, the he invention provides a series of at least two, three, four, five, six, seven, eight, nine, ten or more than ten force sensor peptides, wherein the force sensor peptides in the series have been tuned to undergo a conformation change at least two of: 3, 4, 5, 6, 7, 8, 9, 10, 11, 12, 13, 14, 15, 16, 17, 18, 19, 20, 21, 22, 23, 24, 25 or more than 25 piconewtons (pN).

In some embodiments, the present invention provides methods of use of the force sensing peptides for characterizing proteins. In some embodiments, the method is used to measure the force acting upon a protein of interest. In some embodiments, the method is used to detect protein interactions.

In some embodiments, the present invention provides methods of use of the force sensing peptides to elicit a desired effect when under sufficient force. For example, in some embodiments, the force sensing peptides can be used to provide a detectable signal, to activate a target molecule, or to inhibit a target molecule in the presence of a sufficient level of force to induce a conformational change of the force sensing peptide.

In a variety of embodiments, the present invention provides compositions for utilizing forces acting upon proteins. In a variety of embodiments, the forces acting upon proteins are measured. In some embodiments, the forces acting upon proteins are used to activate the composition. In some embodiments, the composition is used to deliver a target in a force magnitude-dependent manner. In some embodiments, the composition is used to deliver a target in a force frequency-dependent manner. In some embodiments, the composition is used to deliver a target in a force direction-dependent manner. In some embodiments, the composition is used to deliver a target in a cell-specific manner. In some embodiments, the composition is used to deliver a target in an extracellular matrix-dependent manner.

In some embodiments, the invention provides methods for measuring the force acting upon a protein of interest. In some embodiments, the invention provides methods for investigating protein interactions. In some embodiments, the invention provides methods for eliciting a desired effect. In some embodiments, the compositions elicit a desired effect in a spatially, or temporally controlled manner, for example, in some embodiments, the effect is produced in a specific environment under specified conditions (i.e. a sufficient force to induce a conformational change of a force sensor peptide of the invention).

### Compositions

In some embodiments, the invention provides force sensing molecules which provide a detectable output in the presence of a threshold level of force. In some embodiments, the force sensor molecule of the invention comprises a peptide, a glycopeptide, a protein, a protein fragment, a protein domain, a sugar, a polysaccharide, a glycoside, a nucleic acid, a lipid, a glycolipid, a phospholipid, a resin, a metabolite, or a small molecule.

In one aspect, the force sensing molecule of the invention is a force-dependent peptide or protein. In some embodiments, the force-dependent peptide or protein comprises a peptide which undergoes a change when force is applied. The change undergone by the force sensor peptide of the invention can be a change in protein stability, conformation, localization or activation, but is not limited to these types of changes and can be any type of change that can be detected directly or indirectly. In some embodiments, the force dependent peptide or protein comprises a peptide which is inactivated when force is applied. In some embodiments, the peptide or protein comprises a peptide which is exposed to the exterior environment when force is applied. In some embodiments, the force peptide sensor undergoes a conformation change resulting in dissociation of a coiled coil domain to generate an open conformation.

In various embodiments, the composition comprises a force-sensing peptide comprising two α-helix domains or coil elements linked by a flexible linker in such a way that the two α-helix domains can wrap around each other to form a coiled coil.

In some embodiments, the first α-helix domain comprises a peptide sequence of SEQ ID NO:1-20, or a variant thereof. In some embodiments, the second α-helix domain comprises a peptide sequence of SEQ ID NO:21-40, or a variant thereof. In some embodiments, the linker comprises a peptide sequence of SEQ ID NO:41-56, or a variant thereof.

In some embodiments, the first α-helix domain and second α-helix domain are:
a) SEQ ID NO:1 and SEQ ID NO:21;
b) SEQ ID NO:2 and SEQ ID NO: 21;
c) SEQ ID NO:2 and SEQ ID NO: 22;
d) SEQ ID NO:3 and SEQ ID NO: 23;
e) SEQ ID NO:4 and SEQ ID NO:23;
f) SEQ ID NO:4 and SEQ ID NO:24;
g) SEQ ID NO:5 and SEQ ID NO: 25;
h) SEQ ID NO:6 and SEQ ID NO: 26;
i) SEQ ID NO:7 and SEQ ID NO:27,
j) SEQ ID NO:8 and SEQ ID NO:28;
k) SEQ ID NO:9 and SEQ ID NO: 29;
l) SEQ ID NO:10 and SEQ ID NO:30;
m) SEQ ID NO:11 and SEQ ID NO:31;
n) SEQ ID NO:12 and SEQ ID NO:32;
o) SEQ ID NO:13 and SEQ ID NO:33;
p) SEQ ID NO:14 and SEQ ID NO:34;
q) SEQ ID NO:15 and SEQ ID NO:35;
r) SEQ ID NO:16 and SEQ ID NO:36;
s) SEQ ID NO:17 and SEQ ID NO:37;
t) SEQ ID NO:18 and SEQ ID NO:38;
u) SEQ ID NO:19 and SEQ ID NO:39; or
v) SEQ ID NO:20 and SEQ ID NO:40.

Exemplary sensor peptides include, but are not limited to, the peptide sequences set forth in SEQ ID NO:57-80 or variants or fragments thereof. In some embodiments, fragments of the sensor peptides lack the linker sequences (such as GGGG, SEQ ID NO:91) on the C-terminus, the N-terminus or both of SEQ ID NO:57-80.

In some embodiments, the force sensing peptide of the invention is operably linked to one or more functional domain or functional moiety. In some embodiments, the functional domain or functional moiety becomes active when the force sensing peptide is in an open conformation. For example, in some embodiments, the functional domain or functional moiety is incorporated into the linker of the sensor peptide and is unavailable, or inactive when the coiled coil domain is in a closed conformation but becomes exposed (e.g., activated) when the sensor peptide is in an open conformation. For example, in some embodiments, the linker comprises at least one peptide binding domain, RNA binding domain, peptide cleavage domain, small molecule or protein interaction domain, small molecule or protein recognition domain (e.g., a domain comprising FRB/FKBP which form oligomers only in the presence of rapamycin), epitope for binding by a binding molecule (e.g., spyTag/spyCatcher, antigen/nanobody), DNA-binding domain, ion-binding domain, lipid-binding domain, peptide containing residues for post-translational modification, peptide containing unnatural amino acids, peptide that is a toxin to cells, peptide with anti-bacterial activity, peptide with anti-viral activity, peptide with anti-fungal activity, peptide with enzymatic activity, peptide that mediates or modifies the enzymatic activity of other proteins, peptide containing a localization signal for a cellular compartments, a peptide containing a secretion signal, a GFP11 peptide (SEQ ID NO:46), a circularly permutated fluorescent protein (cpFP), a HiBiT peptide (SEQ ID NO:52), an SsrA peptide (SEQ ID NO:53), a TRAP peptide (SEQ ID NOs:54 and 55), a TEV linker (SEQ ID NO:56), a IAAL-K3 (SEQ ID NO:50)/IAAL-E3 (SEQ ID NO:51) binding peptide, a transcription factor, an enzyme (e.g., TurboID), or any combination thereof. In some embodiments, a functional domain or peptide included in the linker becomes active/available/accessible when the force sensing peptide is in an open conformation but is inactive/inaccessible when the force sensing peptide is in a closed conformation.

In some embodiments, the force sensing peptide of the invention is operably linked to at least one target molecule of interest. Examples of target molecules that can be operably linked to or incorporated into the force sensing peptides of the invention include, but are not limited to, a detectable moiety, a fluorescent protein, a bioluminescent protein, a purification tag, a targeting domain, a cellular localization signal, a DNA molecule, an RNA molecule, cAMP, cGMP, eicosapentaenoic acid, eicosatetraenoic acid, a phosphatidylcholine, a phosphatidylinositol, a phosphatidylethanolamine, an aminomethyl polystyrene linker, a chloromethyl polystyrene linker, a PEG linker, a solid-phase protein synthesis resin, a C-terminal fragment of End4p, a C-terminal fragment of actin, a C-terminal fragment of clathrin, a C-terminal fragment of vinculin, a C-terminal fragment of talin, a C-terminal fragment of integrin, glycogen, cellulose, a therapeutic agent, an antibiotic, an antiviral, an anti-fungal, an anti-helminthic, an anti-inflammatory molecule, and a chemotherapeutic.

In some embodiments, the force sensing peptide of the invention is operably linked to at least one target molecule of interest on one end and further linked to at least one molecule to tether the force sensing peptide to a surface or substrate at the other end. Exemplary molecules to tether the force sensing peptide include, but are not limited to, a transmembrane domain or a protein comprising a transmembrane domain, biotin, streptavidin, an antibody, a peptide or epitope for recognition and binding by a binding molecule, or any type of protein or molecule that can be used to tether the force sensor peptide to a surface or substrate.

In some embodiments, the target molecule of interest becomes activated or inactivated when the force sensing peptide is in an open conformation. For example, in some embodiments, the target molecule of interest is operably linked to the N- or C-terminus of the force sensing peptide and is active when the force sensing peptide is in a closed conformation, but is sequestered when the force sensing peptide is in an open conformation due to aggregation of the open conformation of the force sensing peptide. In some embodiments, the target molecule of interest is operably linked to the N- or C-terminus of the force sensing peptide and is tethered when the force sensing peptide is in a closed conformation, but is released when the force sensing peptide is in an open conformation due to exposure of a peptide cleavage sequence in the linker which releases the bound target molecule in the open conformation of the force sensing peptide. In some embodiments, the linker comprises a functional domain surrounded by cleavage sites that can be cut by a protease only when the sensor is in an open conformation. In such an embodiment, the functional domain is released into the cell upon cleavage of the protease cleavage sites. Functional domains include, but are not limited to, a transcription factor, a signaling protein, an enzyme, a toxin, and therapeutic agents. The above exemplary embodiments are provided as examples, and are not intended to limit the application of the force sensing peptide or restrict its use.

In some embodiments, the force sensing peptide of the invention is inserted internally into a target molecule of interest in such a way that the insertion does not alter the function or localization of the target protein when it is in closed conformation. In some embodiments, the conformational change of the force sensing peptide to an open conformation in the presence of a level of force above the unfolding threshold then disrupts the localization or activity of the target protein, providing a detectable change.

In some embodiments, the force sensing peptide of the invention is flanked by a pair of molecules for generating a signal when the force sensing peptide is in an open conformation but not when the force sensing peptide is in a closed formation. Exemplary molecules that can be used to flank the force sensing peptide and provide a signal in an open conformation include, but are not limited to, fluorescent quenching/reporter systems.

In some embodiments, the force sensing peptide of the invention is flanked by a pair of molecules for generating a signal when the force sensing peptide is in a closed conformation but not when the force sensing peptide is in an open formation. Exemplary molecules that can be used to flank the force sensing peptide and provide a signal in a closed conformation include, but are not limited to, FRET donor/acceptor systems.

In a variety of embodiments, a composition of the invention comprises a coiled-coil comprising the first coil element and the second coil element. In some embodiments, the coiled-coil comprises the first coil element and the second coil element in parallel. In some embodiments, the first coil element in the parallel coiled-coil comprises a peptide comprising an amino acid sequence selected from the group consisting of SEQ ID NOs: 1-12, and a variant thereof. In some embodiments, the second coil element in the parallel coiled-coil comprises a peptide comprising an amino acid sequence selected from the group consisting of SEQ ID NOs: 21-32, and a variant thereof. For example, a parallel coiled-coil and the linker between the first and second coil elements may comprise an amino acid sequence of any of SEQ ID NOs: 57-70.

In some embodiments, the coiled-coil comprises the first coil element and the second coil element in anti-parallel. In some embodiments, the first coil element in the anti-parallel coiled-coil comprises a peptide comprising an amino acid sequence selected from the group consisting of SEQ ID NOs: 13-20, and a variant thereof. In some embodiments, the second coil element in the anti-parallel coiled-coil comprises a peptide comprising an amino acid sequence selected from the group consisting of SEQ ID NOs: 33-40, and a variant thereof. For example, an anti-parallel coiled-coil and the linker between the first and second coil elements may comprise an amino acid sequence of any of SEQ ID NOs: 71-80.

In some embodiments, the coiled coils open upon application of a sufficient force and are represented with the force required. Example 6 provides exemplary force sensing peptides, along with their unfolding force threshold. For example, in some embodiments cc-11pN refers to a coiled coil which opens upon application of 11pN of force or more. The force sensing peptide comprising a peptide sequence of SEQ ID NO:57 comprises a cc-11pN.

The peptides of the present invention may be made and combined using chemical methods. For example, peptides can be synthesized by solid phase techniques (Roberge, J. Y., et al., 1995, Science, 269:202-204), cleaved from the resin, and purified by high performance liquid chromatography. Automated synthesis may be achieved, for example, using the ABI 431 A Peptide Synthesizer (Perkin Elmer) in accordance with the instructions provided by the manufacturer.

The peptides of the present invention may be made by biological means and combined using chemical methods.

The peptides of the present invention may be made by biological means already assembled in series as a full final composition.

The peptides of the present invention may be made by biological means already assembled in series as a full protein and loaded with a desired cargo to yield a final composition.

The peptides of the present invention may be made by recombinant means or by cleavage from a longer peptide. The composition of a peptide may be confirmed by amino acid analysis or sequencing.

In some embodiments, the composition comprises variants of the sequences provided. Included in such variants are fragments, which include, but are not limited to, fragments of SEQ ID NO:1-80 comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, and 81 consecutive amino acids of any of SEQ ID NO:1-80.

In some embodiments, the invention comprises any form of a peptide comprising amino acids derived from amino acid sequences of SEQ ID NOs: 1-80. In certain embodiments, the invention includes amino acids sequences having substantial homology to peptides of amino acid sequences SEQ ID NOs: 1-80. In some embodiments, a peptide which is “substantially homologous” is about 50% homologous, about 70% homologous, about 80% homologous, about 85% homologous, about 90% homologous, about 91% homologous, about 92% homologous, about 93% homologous, about 94% homologous, about 95% homologous, about 96% homologous, about 97% homologous, about 98% homologous, about 99% homologous to amino acid sequences of SEQ ID NOs: 1-80.

As known in the art, the “similarity” between two peptides is determined by comparing the amino acid sequences and its conserved amino acid substitutions of one polypeptide to a sequence of a second polypeptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present invention includes, but is not limited to, amino acid sequences that are at least 60%, 65%, 70%, 72%, 74%, 76%, 78%, 80%, 82%, 84%, 86%, 88%, 90%, 91%, 92%, 93%, 94%, 95%, 96%, 97%, 98%, or 99% similar or identical to the amino acid sequence. The degree of identity between two peptides is determined using computer algorithms and methods that are widely known for persons skilled in the art. The identity between two amino acid sequences is preferably determined by using the BLASTP algorithm (BLAST Manual, Altschul, S., et al., NCBI NLM NIH Bethesda, MD 20894, Altschul, S., et al., 1990, J. Mol. Biol., 215:403-410).

The peptides of the invention can be post-translationally modified. For example, post-translational modifications that fall within the scope of the present invention include signal peptide cleavage, glycosylation, acetylation, isoprenylation, proteolysis, myristoylation, phosphorylation, protein folding and proteolytic processing, etc. Some modifications or processing events require introduction of additional biological machinery. For example, processing events such as signal peptide cleavage and core glycosylation are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during the protein translation or synthesis.

A peptide or protein of the invention may be conjugated with other molecules, such as proteins, to prepare fusion proteins. This may be accomplished, for example, by the synthesis of N-terminal or C-terminal fusion proteins provided that the resulting fusion protein remains the functionality of force-activated composition.

A peptide or protein of the invention may be phosphorylated using conventional methods such as the method described in Reedjik et al. (1992, The EMBO Journal, 11:1365).

In some embodiments, the present invention provides a composition comprising an isolated nucleic acid encoding a force-dependent peptide or protein. In various embodiments, the isolated nucleic acid sequence encodes a first coil element conjugated, at its N-terminus, to a peptide linker, which is in turn conjugated, at its N-terminus, to a second coil element. In some embodiments, the isolated nucleic acid sequence encodes a first moiety, conjugated at its N-terminus, to a first coil element conjugated, at its N-terminus, to a peptide linker, which is in turn conjugated, at its N-terminus, to a second coil element. In some embodiments, the isolated nucleic acid sequence encodes a first moiety, conjugated at its N-terminus, to a first coil element conjugated, at its N-terminus, to a peptide linker, which is in turn conjugated, at its N-terminus, to a second coil element, conjugated at its N-terminus, to a second moiety.

In some embodiments, the isolated nucleic acid sequence encodes a peptide or protein comprising a first coil element comprising an amino acid sequence selected from the group consisting of SEQ ID NO:1-20, and variants thereof. In some embodiments, the isolated nucleic acid sequence encodes a peptide or protein comprising a linker comprising an amino acid sequence selected from the group consisting of SEQ ID NO:41-56. In some embodiments, the isolated nucleic acid sequence encodes a peptide or protein comprising a second coil element comprising an amino acid sequence selected from the group consisting of SEQ ID NO:21-40. In some embodiments, the isolated nucleic acid sequence encodes a peptide or protein comprising an amino acid sequence selected from the group consisting of SEQ ID NO:57-80.

Further, the invention encompasses an isolated nucleic acid encoding a peptide having substantial homology to a peptide comprising an amino acid sequence selected from the group consisting of SEQ ID NO:1-80. In certain embodiments, the isolated nucleic acid sequence encodes a peptide having at least 75%, 80%, 85%, 90%, 91%, 92%, 93%, 94%, 95%, 96%, 97%, 98%, or 99% sequence homology with an amino acid sequence selected from SEQ ID NOs:1-80.

The isolated nucleic acid sequence may comprise any type of nucleic acid, including, but not limited to DNA and RNA. For example, In some embodiments, the composition comprises and isolated DNA molecule, including for example, an isolated cDNA molecule, encoding a coiled-coil of SEQ ID NO:1-80. In some embodiments, the composition comprises an isolated RNA molecule encoding a coiled-coil of SEQ ID NO:1-80.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogs, e.g., substitution of uridine by 2′-deoxythimidine is tolerated and does not affect function of the molecule.

In some embodiments of the present invention, the nucleic acid molecule may contain at least one modified nucleotide analog. For example, the ends may be stabilized by incorporating modified nucleotide analogs.

Non-limiting examples of nucleotide analogs include sugar- and/or backbone-modified ribonucleotides (i.e., include modifications to the phosphate sugar backbone). For example, the phosphodiester linkages of natural RNA may be modified to include at least one of a nitrogen or sulfur heteroatom. In preferred backbone modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH2, HNR, NR2, or ON, wherein R is C₁-C₆ alkyl, alkenyl, or alkynyl and halo is F, Cl, Br, or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytodine modified at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-dadenosine; O- and N-alkylated nucleotides, e.g., N6-methyl adenosine are suitable. It should be noted that the above modifications may be combined.

In some instances, the nucleic acid molecule comprises at least one of the following chemical modifications: 2′H, 2′-O-methyl, or 2′-OH modification of one or more nucleotides. In certain embodiments, a nucleic acid molecule of the invention can have enhanced resistance to nucleases. For increased nuclease resistance, a nucleic acid molecule can include, for example, 2-modified ribose units and/or pohsphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-Omethoxyethyl, 2′-O-aminopropyl, 2′-amino, and/or phosphorothioate linkages. Inclusion of locked nucleic acids (LNA), ethylene nucleic acids (ENA), e.g., 2′-4′-ethylenebridged nucleic acids, and certain nucleobase modifications such as 2-amino-A, 2-thio (e.g., 2-thio-U), G-clamp modifications, can also increase binding affinity to a target. In some embodiments, the nucleic acid molecule includes a 2′-modified nucleotide, e.g., a 2′-deoxy, 2′-deoxy-2′-fluoro, 2′-O-methyl, 2′-O-methoxyethyl (2′-O MOE), 2′-O-aminopropyl (2′-O-AP), 2′-O-dimethylaminoethyl (2′-O-DMAOE), 2′-Odimethylaminopropyl (2′-O-DMAP), 2′-O-dimethylaminoethyloxyethyl (2′-ODMAEOE), or 2′-O—N-methylacetamido (2′-O-NMA). In some embodiments, the nucleic acid molecule includes at least one 2′-O-methyl-modified nucleotide, and in some embodiments, all of the nucleotides of the nucleic acid molecule include a 2′-O-methyl modification.

In certain embodiments, the nucleic acid molecule of the invention preferably has one or more of the following properties:

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, preferably different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of: a phosphate group, a sugar group, the backbone, 5′-end, the 3′-end, or nucleobase.

The present invention also includes a vector in which the isolated nucleic acid of the present invention is inserted. The art is replete with suitable vectors that are useful in the present invention.

In brief summary, the expression of natural or synthetic nucleic acids encoding coiled-coil peptide is typically achieved by operably linking a nucleic acid encoding the coiled-coil peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid.

Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In some embodiments, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

In some embodiments, a replication-deficient adenovirus can be used. In some embodiments, replication-deficient serotype 5 adenovirus can be used.

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In some embodiments, the vector of the present invention comprises one or more enhancers to boost transcription of the gene present within the vector.

In order to assess the expression of a peptide or protein, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Genetically Modified Cells

Exogenous genetic material (e.g., a nucleic acid molecule encoding a force sensor peptide) can be introduced into a host cell in vivo by genetic transfer methods, such as transfection or transduction, to provide a genetically modified cell. Various expression vectors (i.e., vehicles for facilitating delivery of exogenous genetic material into a target cell) are known to one of ordinary skill in the art.

As used herein, “transfection of cells” refers to the acquisition by a cell of new genetic material by incorporation of added DNA. Thus, transfection refers to the insertion of nucleic acid into a cell using physical or chemical methods. Several transfection techniques are known to those of ordinary skill in the art including: calcium phosphate DNA co-precipitation; DEAE-dextran; electroporation; cationic liposome-mediated transfection; and tungsten particle-facilitated microparticle bombardment. Strontium phosphate DNA co-precipitation is another possible transfection method. In contrast, “transduction of cells” refers to the process of transferring nucleic acid into a cell using a DNA (e.g., an adenovirus) or RNA virus (e.g., a retrovirus).

In some embodiment the host cell that has been transfected or transduced with an expression vector for expression of the force sensor peptide of the invention will not have the exogenous genetic material incorporated into its genome, but will be capable of expressing the exogenous genetic material that is retained extrachromosomally within the cell. In some embodiment the host cell that has been transfected or transduced with an expression vector for expression of the force sensor peptide of the invention will have the exogenous genetic material incorporated into its genome. Incorporation of genetic material into the genome of a host cell can be non-specific (e.g., retroviral based recombination) or can be directed by site-specific recombination methods (e.g., homology directed recombination or CRISPR/Cas9 recombination).

In some embodiments, the exogenous genetic material includes a nucleotide sequence encoding a force sensor peptide construct of the invention together with a promoter to control transcription of the force sensor peptide construct. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion, an “enhancer” is simply any non-translated DNA sequence that works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. An expression vector may further include an exogenous promoter element to control transcription of the force sensor peptide construct following insertion of the construct into a host cell genome. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes that encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eucaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a force sensor construct in the genetically modified cell. If the gene encoding the therapeutic agent is under the control of an inducible promoter, delivery of the therapeutic agent in situ is triggered by exposing the genetically modified cell in situ to conditions for permitting transcription of the force sensor construct, e.g., by intraperitoneal injection of specific inducers of the inducible promoters which control transcription of the force sensor construct. For example, in situ expression by genetically modified cells of a force sensor construct encoded by a gene under the control of the metallothionein promoter, is enhanced by contacting the genetically modified cells with a solution containing the appropriate (i.e., inducing) metal ions in situ.

In addition to at least one promoter and at least one heterologous nucleic acid encoding the force sensor construct, the expression vector may include a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation.

A force sensor construct of the present invention can be inserted into any type of target or host cell. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New 20 York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR, RT-qPCR and PCR; “biochemical” assays, such as detecting the presence or absence of a particular peptide, e.g., by immunological means (ELISAs and Western blots) or by assays described herein to identify agents falling within the scope of the invention.

### Methods of Measuring Force

In some embodiments, the force sensing molecules of the invention are operably linked to a target protein such that they undergo a change or produce a signal, or a combination thereof, when force is applied to the target protein of interest. In some embodiments, the signal is a detectable signal that can be detected directly or indirectly. Exemplary detectable signals include, but are not limited to, aggregation, fluorescent signals, and electrochemical signals. Detection of a change or signal of the sensor protein is an indicator that the force sensor protein was put under sufficient force to induce the change or signal. For example, in some embodiments, the sensor protein undergoes a change or generates a signal if acted upon by at least 3 pN of force, therefore detection of the change or signal would indicate that the sensor protein experienced at least 3 pN of force. In some embodiments, the sensor protein undergoes a change or generates a signal if acted upon by at least 5 pN of force, therefore detection of the change or signal would indicate that the sensor protein experienced at least 5 pN of force. In some embodiments, the sensor protein undergoes a change or generates a signal if acted upon by at least 7 pN of force, therefore detection of the change or signal would indicate that the sensor protein experienced at least 7 pN of force. In some embodiments, the sensor protein undergoes a change or generates a signal if acted upon by at least 10 pN of force, therefore detection of the change or signal would indicate that the sensor protein experienced at least 10 pN of force. In some embodiments, the sensor protein undergoes a change or generates a signal if acted upon by at least 11 pN of force, therefore detection of the change or signal would indicate that the sensor protein experienced at least 11 pN of force. In some embodiments, the sensor protein undergoes a change or generates a signal if acted upon by at least 14 pN of force, therefore detection of the change or signal would indicate that the sensor protein experienced at least 14 pN of force. In some embodiments, the sensor protein undergoes a change or generates a signal if acted upon by at least 18 pN of force, therefore detection of the change or signal would indicate that the sensor protein experienced at least 18 pN of force. In some embodiments, the sensor protein undergoes a change or generates a signal if acted upon by at least 20 pN of force, therefore detection of the change or signal would indicate that the sensor protein experienced at least 20 pN of force.

In some embodiments, the present invention provides a method for the measurement of a force applied to a protein. In some embodiments, when a sufficient force is applied to a peptide or protein of the invention, a force-responsive domain opens, exposing the linker to the environment. In some embodiments, when the linker is exposed to the environment, proteins or peptides aggregate to form visible condensates. In some embodiments, when the linker is exposed to the environment, a GFP11 domain is exposed which interacts with soluble GFP1-10, providing a visible readout. In some embodiments, when the force-responsive domain opens, the confirmation of the linker changes such that it modifies the photo-physical properties of a circularly permutated fluorescent protein inserted in the linker, allowing for absorption and/or emission at a different wavelength than when the coiled coil domain is in a closed conformation. In some embodiments, when the linker is exposed to the environment, a HiBiT domain is exposed which interacts with a soluble LgBiT, providing a visible readout. In some embodiments, when the linker is exposed to the environment, a small stable RNA A (SsrA) domain is exposed, which interacts with Stringent starvation protein B (SspB), to recruit SspB-tagged molecules. In some embodiments, when the linker is exposed to the environment, a tetratricopeptide repeat affinity protein (TRAP) peptide is exposed, which interacts with a fluorescently-tagged TRAP, providing a visible readout. In some embodiments, when the linker is exposed to the environment, a tobacco etch virus (TEV) recognition linker is exposed, which is cleaved by a TEV protease.

In some embodiments, two or more force sensing peptides are used in parallel, or alternatively in tandem if each of the two or more force sensing peptides produces a different signal, wherein each of the two or more force sensing peptides has a different uncoiling threshold, allowing for determination of the force exerted on the target to be determined within a narrow range. For example, when the force a protein of interest is exposed to is insufficient, the force-responsive domain does not open, the linker is not exposed, and there is no detectable signal. For other force-responsive domains the force is great enough, the force-responsive domain opens, the linker is exposed, and there is a positive detectable signal. The force applied to the protein is then determined to be greater than the highest-open conformation composition and less than the lowest-closed conformation composition. For example, if the highest-open conformation composition comprises a coiled coil domain that is responsive (uncoils) at 10 pN (cc-10pN) and the lowest-closed conformation composition comprises a a coiled coil domain that is responsive (uncoils) at 11 pN (cc-11pN), then the force applied to the protein of interest is determined to be between 10 and 11 pN.

### Methods of Eliciting an Effect in a Force-Dependent Manner

In some embodiments, the composition of the invention produces a desired effect when force is applied to a protein. In some embodiments, the present invention provides a method for producing a desired effect when a sufficient force is applied to a protein. In some embodiments, when a sufficient force is applied to a peptide or protein of the invention, the force-responsive domain opens, and the linker is exposed to the environment. In some embodiments, the linker comprises an RNA-binding domain, which is exposed to the environment when a sufficient force is applied. In some embodiments, an RNA molecule is made accessible upon exposure of the RNA-binding domain. In some embodiments, the RNA molecule is a ribozyme. In some embodiments, the RNA molecule is a shRNA. In some embodiments, the RNA molecule is an miRNA. In some embodiments, the RNA molecule is an siRNA. For example, upon exposure of a sufficient force to a composition of the invention, the coiled-coil opens, exposing an RNA-binding domain, which binds to a target mRNA molecule thereby reducing the mRNA levels of a protein of interest.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out certain embodiments of the present invention, and are not to be construed as limiting in any way the remainder of the disclosure.

### Example 1: Force Redistribution in Clathrin-Mediated Endocytosis

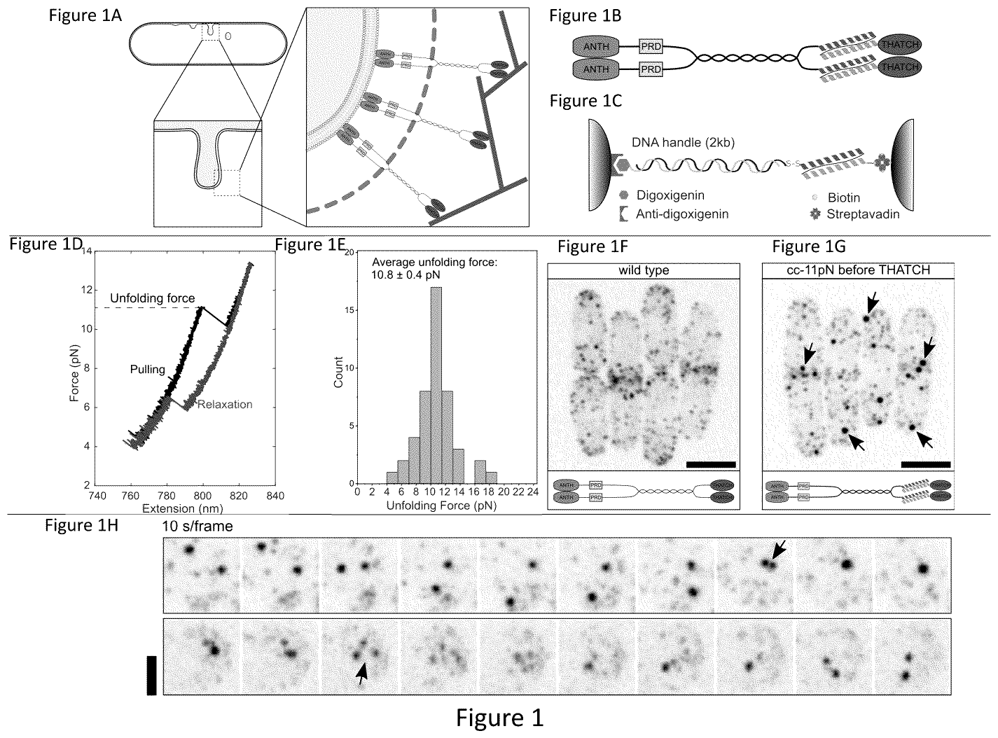

Extracellular materials are transported into cells via endocytosis. In eukaryotic cells, clathrin-mediated endocytosis (CME) is the major internalization pathway for nutrients, signaling molecules and pathogenic agents (Mettlen, M., et al., 2018, Annual Review of Biochemistry, 87:871-896; Kaksonen, M. et al., 2018, Nat Rev Mol Cell Biol, 19:313-326). It is implicated in numerous diseases including cancer, neurological disorders and virus entry, and is therefore the focus for both basic and translational research (McMahon, H. T., et al., 2011, Nat Rev Mol Cell Biol, 12:517-533). The distinctive feature of CME is a layer of proteinaceous coat, of which clathrin is a prominent member. The coat contains more than 20 evolutionarily conserved proteins that assemble at the intracellular side of the endocytic site, curves as the endocytic pit matures, and disassembles after the budding of the endocytic vesicle (Kukulski, W., et al., 2012, Cell, 150:508-520; Sun, Y., et al., 2019, eLife, 8:e50749). Endocytic coat proteins make extensive interactions with each other, with the lipids of the plasma membrane, and with a meshwork of actin filaments that surrounds the endocytic coat (Skruzny, M., et al., 2020, Molecular Systems Biology, 16:e9009; Legendre-Guillemin, V., 2004, Journal of Cell Science, 117:9-18; Baggett, J. J., et al., 2003, Genetics, 165:1661-1674; Maldonado-Báez, L., et al., 2006, Trends in Cell Biology, 16:505-513). Forces produced by actin polymerization are transmitted through adaptor proteins of the endocytic coat to help transform a flat membrane patch into a cargo-filled vesicle into the cytoplasm (Skruzny, M., et al., 2012, Proc Natl Acad Sci USA, 109:E2533-E2542; Skruzny, M., 2015, Developmental Cell, 33:150-162; Lacy, M., et al., 2018, FEBS Letters, 592:3586-3605) (FIG. 1A).

Deformation of the membrane during CME is energetically expensive (Lacy, M., et al., 2018, FEBS Letters, 592:3586-3605; Ma, R. et al., 2021, Biophys J, 120:1625-1640; Basu, R., et al., 2014, Mol Biol Cell, 25:679-687). In mammalian cells with elevated membrane tension and in yeast cells where the turgor pressure is as high as 1 MPa, actin polymerization is required for successful CME (Kukulski, W., et al., 2012, Cell, 150:508-520; Basu, R., et al., 2014, Mol Biol Cell, 25:679-687; Aghamohammadzadeh, S., et al., 2009, Nat Cell Biol, 11:1039-1042). Epsins and Hip1R are believed to transmit the force produced by actin assembly to the plasma membrane because their removal stalls the invagination of endocytic pits, and their C-terminal domains (ACB and THATCH, respectively) bind actin filaments and their N-terminal domains (ENTH and ANTH, respectively) bind PIP2 on the membrane (Baggett, J. J., et al., 2003, Genetics, 165:1661-1674; Skruzny, M., et al., 2012, Proc Natl Acad Sci USA, 109:E2533-E2542; Skruzny, M., 2015, Developmental Cell, 33:150-162; Messa, M., et al., 2013, eLife. 3:e03311) (FIG. 1). Because both proteins also bind clathrin and other endocytic coat proteins, it is possible that forces could be transmitted to the membrane via multiple routes (Skruzny, M., et al., 2020, Molecular Systems Biology, 16:e9009; Aguilar, R. C., et al., 2003, J. Biol. Chem, 278:10737-10743; Engqvist-Goldstein, A. E. Y., et al., 2001, J Cell Biol, 154:1209-1224; Wilbur, J. D., et al., 2008, J Biol Chem, 283:32870-32879). In contrast to the abundant knowledge gleaned from biochemical and genetic approaches, a quantitative understanding of force production and distribution at the molecular level during CME is lacking, because tools to measure forces in live cells in the context of small (˜100-nm diameter) and transient (˜10 s) endocytic pits are scarce, difficult to use, and often too bulky to insert into proteins without causing side effects.

The assembly of the endocytic coat relies on weak and multivalent interactions that facilitate the rapid exchange of binding partners during the dynamic rearrangement of the endocytic coat and the constant change in membrane shape during CME (Smith, S. M., et al., 2017, Front. Mol. Biosci, 4:e00072; Garcia-Alai, M. M., et al., 2018, Nature Communications, 9:328; Zhuo, Y., et al., 2015, Biochemistry, 54:2571-2580). Many endocytic proteins contain promiscuous binding sites, multiple copies of short peptide motifs, and intrinsically disordered regions (IDRs) (Maldonado-Báez, L., et al., 2006, Trends in Cell Biology, 16:505-513; Smith, S. M., et al., 2017, Front. Mol. Biosci, 4:e00072; Garcia-Alai, M. M., et al., 2018, Nature Communications, 9:328; Miao, Y., et al., 2018, The FEBS Journal, 285:2762-2784). Recent developments in protein engineering have indicated that the dual incorporation of IDRs and oligomerization domains (either controlled by light or small molecules) promotes the phase separation of proteins in vivo (Bracha, D., et al., 2019, Nature Biotechnology, 37:1435-1445; Bracha, D., et al., 2018, Cell, 175, 1467-1480.e13; Nakamura, H., et al., 2018, Nat Mater, 17:79-89). If a mechanically actuated oligomerization domain is introduced into an endocytic adaptor protein that contains an IDR, protein phase separation could be induced in a force-dependent manner.

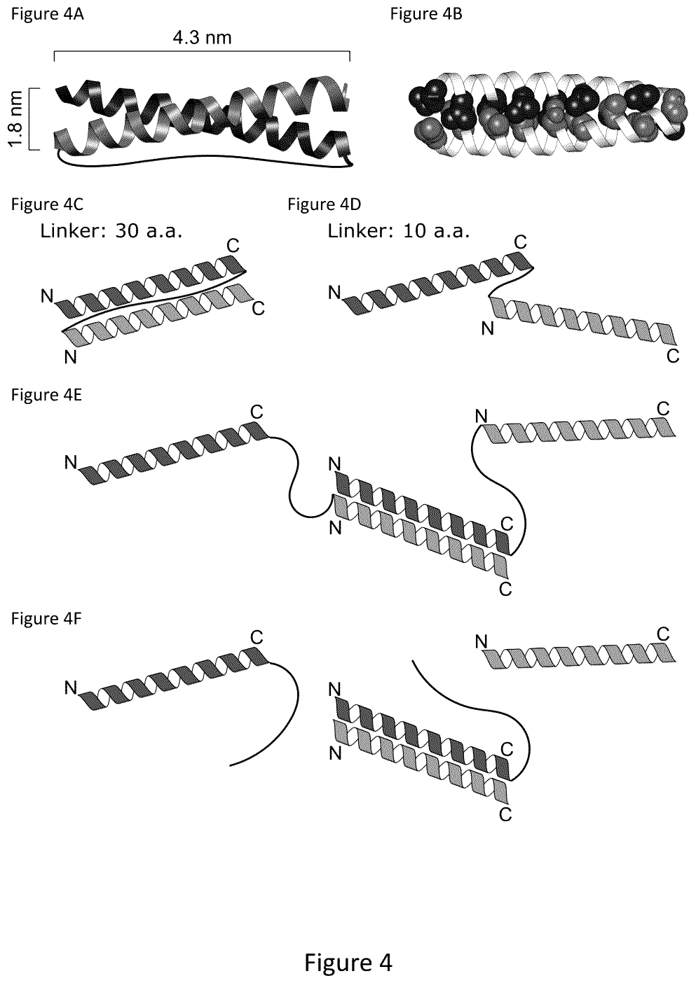

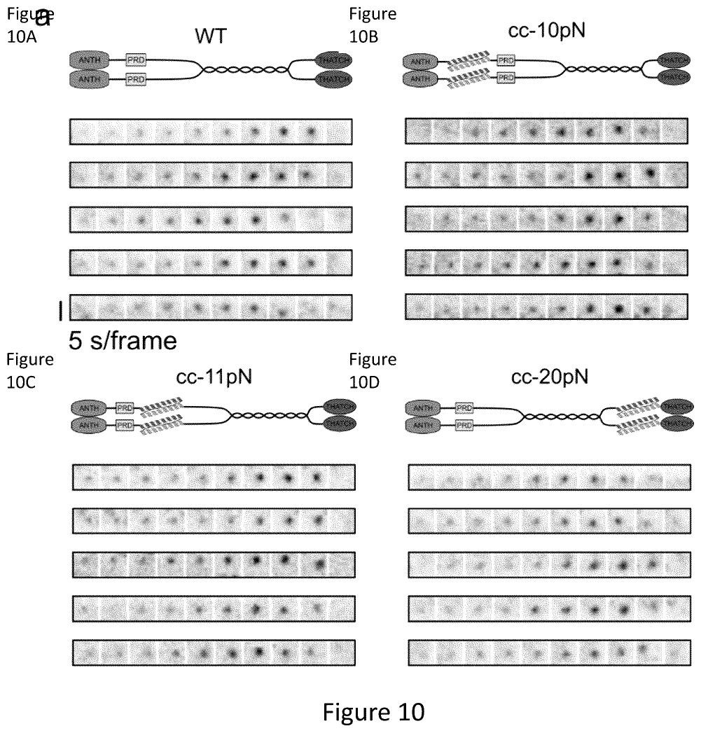

Dimeric coiled-coils are excellent candidates for such mechanically actuated oligomerization domain, because they are small, have well-characterized shape and mechanical properties, and can be easily introduced into proteins without affecting their normal functions (Tanenbaum, M. E., et al., 2014, Cell. 159, 635-646; Truebestein, L., et al., 2016, Bioessays, 38:903-916; Lebar, T., et al., 2020, Nat Chem Biol, 16:513-519) (FIG. 4A, FIG. 10). The unfolding forces and energies of dimeric coiled-coils have previously been measured, and it was found that they unfold at pulling force thresholds in the range of 2-14 piconewtons (pN) (Goktas, M., et al., 2018, Chem. Sci., 9:4610-4621; Gao, Y., et al., 2011, J. Am. Chem. Soc., 133:12749-12757; Xi, Z., et al., 2012, PNAS, 109:5711-5716). The unfolding of a coiled-coil exposes two hydrophobic interaction surfaces that can mediate higher order oligomerization (Pandya, M. J., et al., 2000, Biochemistry, 39:8728-8734) (FIGS. 4B and 4E), while a folded coiled-coil puts an upper limit to the local force magnitude and serves as a control for the insertion.

Force requirement in CME has been the subject of multiple theoretical work, but direct force measurements in the densely woven coat of CME have been lacking (Lacy, M., et al., 2018, FEBS Letters, 592:3586-3605; Dmitrieff, S., et al., 2015, PLOS Computational Biology, 11:e1004538; Nickaeen, M., et al., 2019, bioRxiv, 518423). Only one recent study in S. cerevisiae used FRET-based force sensors to measure the forces on the budding yeast homolog of End4p, Sla2, in a mutant background (Abella, M., et al., 2021, Developmental Cell, 56:2419-2426.e4). This study measured forces larger than 8 pN near the THATCH actin-binding domain. Calibrated coiled-coils were developed as novel force sensors to induce force-dependent phase separation of the endocytic adaptor End4p. Further, it was discovered that, in a wild-type background, the forces directly transmitted by the actin network to End4p C-terminal end are between 11 and 20 pN. There is a gradient of force along End4p, demonstrating forces are relayed and redistributed across the endocytic coat.

This strategy to measure forces in live cells harnesses the pre-existing multivalent interactions in protein complexes and circumvents the need for inserting two large fluorescent proteins in the middle of the target protein as prescribed in approached using FRET force sensors. These force sensors are significantly smaller also because the effector of force, i.e., the calibrated coiled-coils, and the fluorescence reporters are physically separated. The self-propagating intermolecular interactions of the coiled-coil force sensors are triggered by transient force on End4p, and the effect of force is amplified both spatially and temporally through the generation of condensates that have micrometer diameters and outlast the lifetime of the endocytic coat. The combination of the small size and the clear readout makes this coiled-coil based strategy a useful and novel tool for easy in vivo force measurements in previously inaccessible locations with standard fluorescence microscopes. Another advantage of this strategy is that it embeds internal controls and redundancies since all sensors have very similar folds. After insertion into the same position within a protein, a folded coiled-coil force sensor detects the upper limit of the local force magnitude without changing the protein function, while an unfolded coiled-coil force sensor reports the lower limit of force and drives the formation of protein condensates for easy detection. Pairwise application of the coiled-coil force sensors demarcates the force range. These calibrated coiled-coils are compatible with the FRET approach (Ringer, P., et al., 2017, Nature Methods, 14:1090-1096) and complement nicely the FRET based strategy especially in situations where the insertion of the large FRET pair is not tolerated.

It is not expected that the coiled-coil force thresholds measured in vitro to be significantly different in vivo. First, the coiled-coils are derived from prokaryotic proteins or were artificially designed, and no proteins are known to interact with them, so their unfolding properties are unlikely to be changed in vivo. Second, the in vivo phenotypes are always consistent with the order of the calibrated coiled-coils (e.g., if the cc-11pN construct aggregates, the cc-10pN and cc-8pN constructs also aggregate). Third, they are small and they are inserted into unstructured regions that are unlikely to interfere with the protein's function. Fourth, even though the rate of pulling on the protein in the subcellular context and in vitro may be different and may affect the opening force thresholds, the rate used in vitro is in the same range as what is expected during most cellular processes including clathrin-mediated endocytosis in yeast, and previous data and theory strongly suggest the effect of pulling rate on the force threshold is small (less than 30% for a 10-fold faster rate).

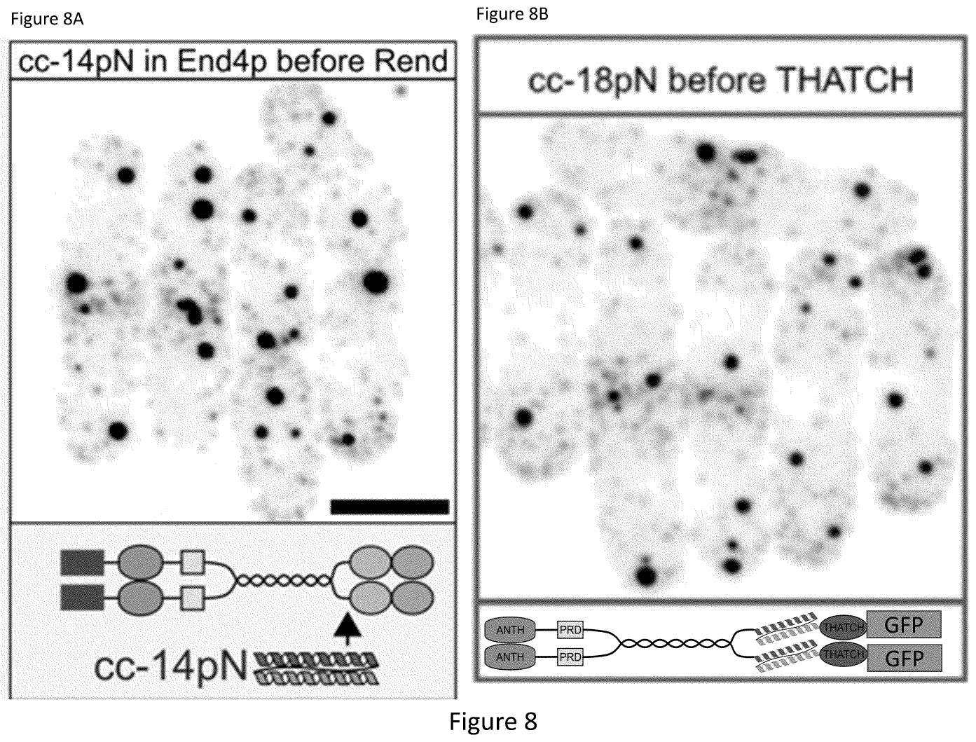

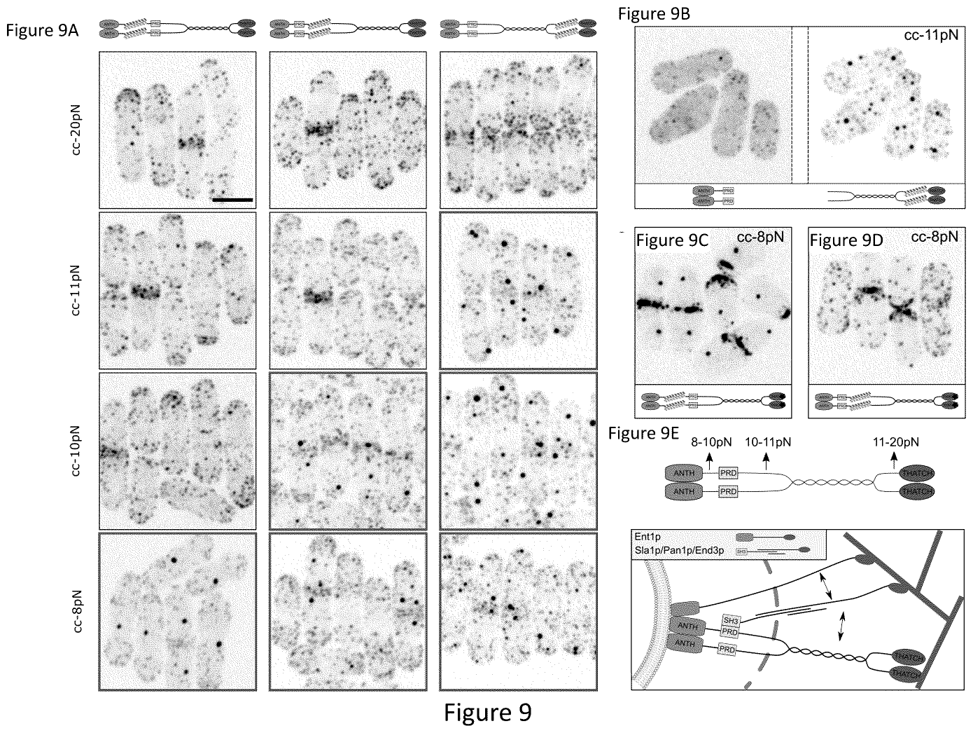

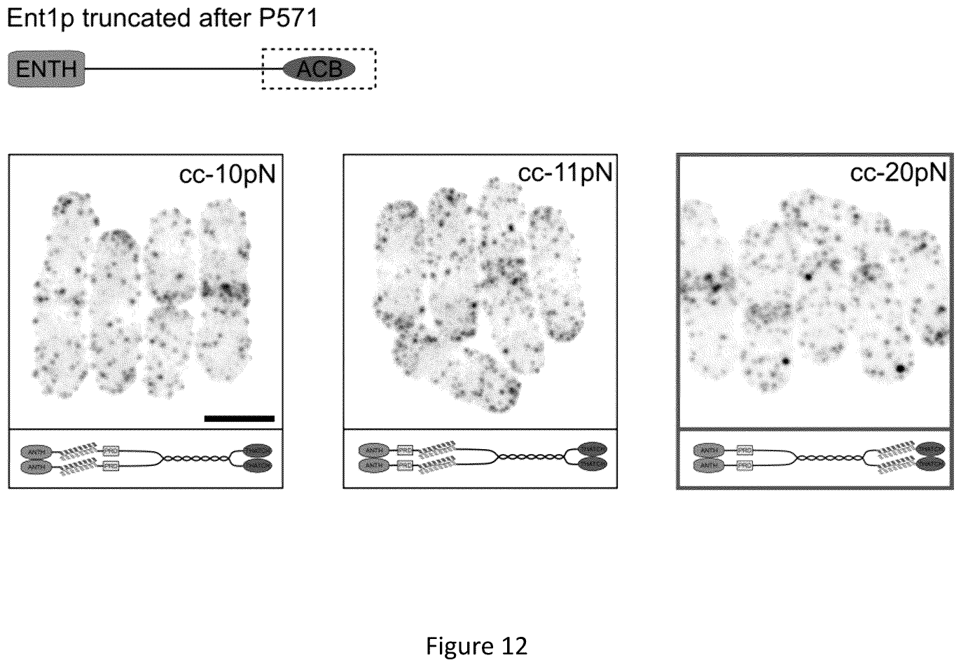

It is estimated that the force on each End4p molecule to is in the 8-10 pN range after the lipid-binding ANTH domain, in the 10-11 pN range between the proline rich domain and the dimerization domain, and in the 11-20 pN range before the THATCH domain (FIG. 9E). The gradient of force along End4p and the mutant data showing that force can still be transmitted to the membrane even if End4p cannot bind actin strongly suggest that the endocytic forces are integrated along the endocytic coat according to a “collect-and-redistribute” mechanism. Since previous quantitative microscopy studies showed there are up to ˜120 End4p molecules per endocytic coat (Sun, Y., et al., 2019, eLife, 8:e50749), the data suggest that forces in the 1320-2400 pN range are generated by the actin meshwork on End4p at the periphery of the endocytic coat, and at least 960-1200 pN is transmitted directly to the lipid membrane by End4p ANTH domain. These forces on End4p are smaller but in the same order of magnitude as the total endocytosis forces predicted by theory. Total forces on the plasma membrane are likely larger since force transmission in the endocytic coat is relayed through the binding to other adaptor proteins, as fragments of End4p that do not bind actin are still under tension in the endocytic coat (FIGS. 8B-8C). Sla1p/Pan1p/End3p protein complex, which arrives later than End4p in the assembly of the endocytic coat and has been shown to have interactions with both End4p and Ent1p, bridges the transmission of force from F-actin to End4p (FIG. 9E) (Sun, Y., et al., 2015, Molecular Biology of the Cell, 26:3841-3856; Tang, H.-Y., et al., 2000, Molecular and Cellular Biology, 20:12-25). The clathrin lattice is probably a hub for integrating the transmission of force in the endocytic coat, as End4p, Ent1p, Sla1p and numerous other endocytic adaptor proteins bind to the clathrin lattice, and change in the magnitude of force on End4p was detected before and after its dimerization domain, which contains the putative clathrin binding site (Engqvist-Goldstein, A. E. Y., et al., 2001, J Cell Biol, 154:1209-1224; Wilbur, J. D., et al., 2008, J Biol Chem, 283:32870-32879). The heavily interconnected endocytic coat ensures redundancy to robustly transmit forces deep into the endocytic coat and to the membrane, despite peripheral perturbations closer to the F-actin binding side (FIG. 12). Magnitudes of forces during CME are slightly smaller in budding yeast because turgor pressure there is slightly lower, and the forces are even smaller in mammalian cells where turgor pressure is several orders of magnitude lower. The redundancy in force transmission through the binding of endocytic coat proteins, however, is probably conserved.

The active involvement of protein phase separation during natural cellular processes is increasingly appreciated. Force produced by the cytoskeleton or motor proteins may change the folding of protein domains to promote or inhibit protein phase separation. The phase separation of LIMD1 recruits focal adhesion proteins in a force dependent manner (Wang, Y., et al., 2021, Dev Cell, 56:1313-1325.e7). The prevalence of low-affinity interactions in the endocytic coat suggests the potential involvement of protein phase separation in normal CME (Kozak, M., et al., Preprint 2019, eLife, 11:e72865; Day, K. J., et al., 2021, Nat Cell Biol, 23:366-376; Bergeron-Sandoval, L.-P., et al., 2021, bioRxiv, 145664), which might be more transient in the natural state but seems to be enhanced by the insertion of the coiled-coil force sensors within End4p.

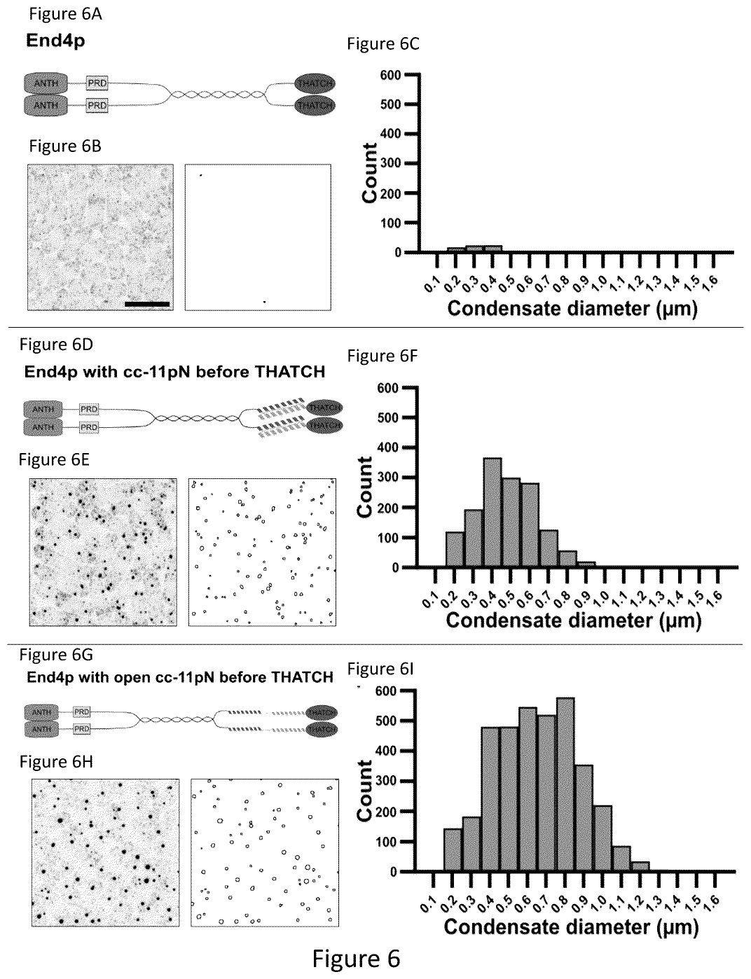

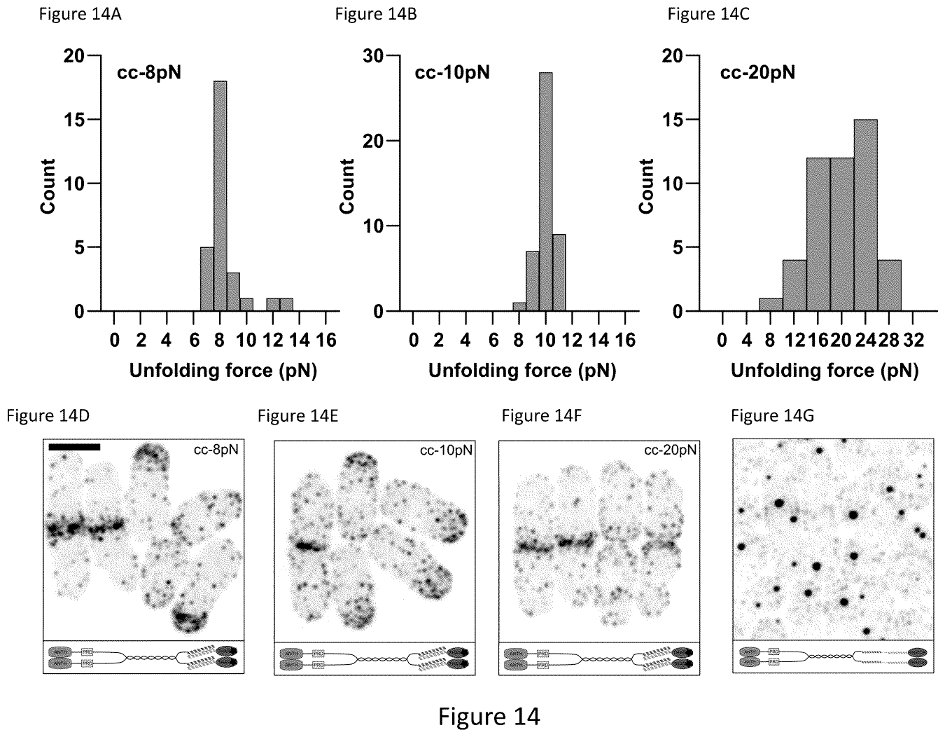

Although the focus of this was not on the in vitro characterization of the phase-separating properties of End4p proteins with coiled-coil insertions, in vivo data offer a clear mechanism for the phase separation of End4p constructs. Valency amplification from zero to two following the unfolding of coiled-coils is critical for generating End4p condensates (FIG. 6, FIG. 14G), and that the disconnection of two α-helices, which prevents intermolecular interactions, disrupts End4p condensates. These coiled-coil force sensors therefore represent a new way to induce in vivo protein condensates in a force-dependent manner, joining currently existing approaches using light or small molecules. The ample knowledge related to the design and engineering of coiled-coils pave the road for additional functionalities based on this simple protein motif.

### Design and Calibration of Cc-11pN

First the two parallel α-helices in a heterodimeric GCN4 leucine zipper were linked with a 30-amino acid flexible linker to form a single chain polypeptide that can be genetically encoded (FIG. 4A). To measure its unfolding force using optical tweezers, a single GCN4 coiled-coil was tethered between two beads held in two optical traps (FIG. 1C) and pulled to high force by separating the two traps at a speed of 10 nm/s (Gao, Y., et al., 2011, J. Am. Chem. Soc., 133:12749-12757), which is the typical predicted speed of actin polymerization during endocytosis. Unfolding of the coiled-coil was manifested by a sudden extension increase at ˜11 pN (FIG. 1D, black trace). The unfolded coiled-coil refolded during relaxation, but at a lower force (FIG. 1D, red trace). Repeated pulling and relaxation reveals a distribution of unfolding forces, with a single peak at 10.8±0.4 pN (Mean±SEM) (FIG. 1E). This coiled-coil is referred to as cc-11pN hereafter.

### Force Applied to End4p Induces Condensation

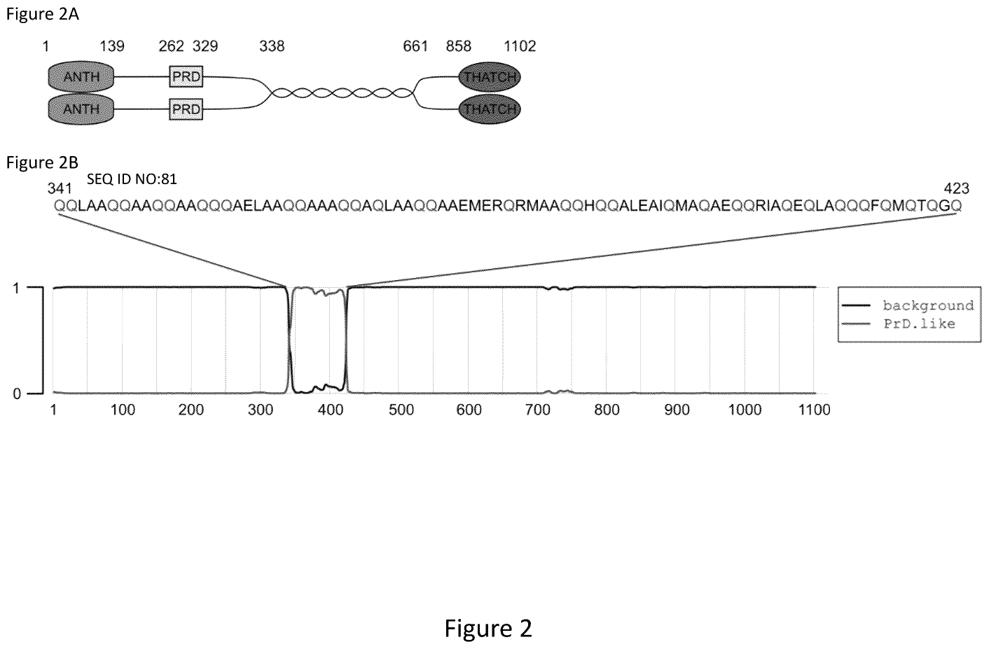

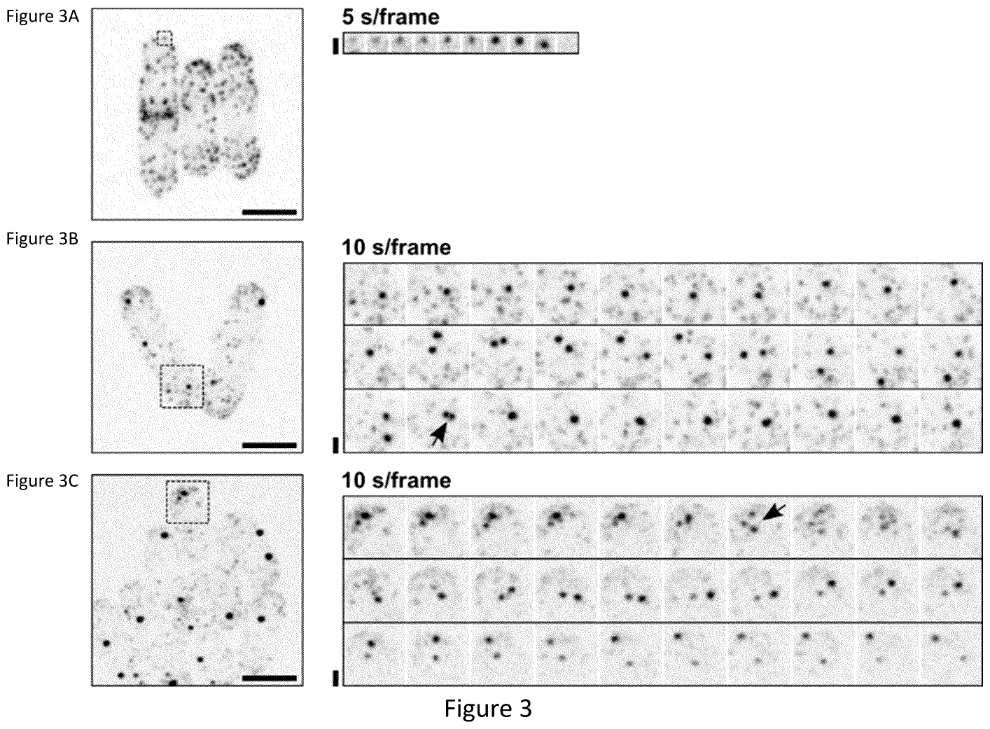

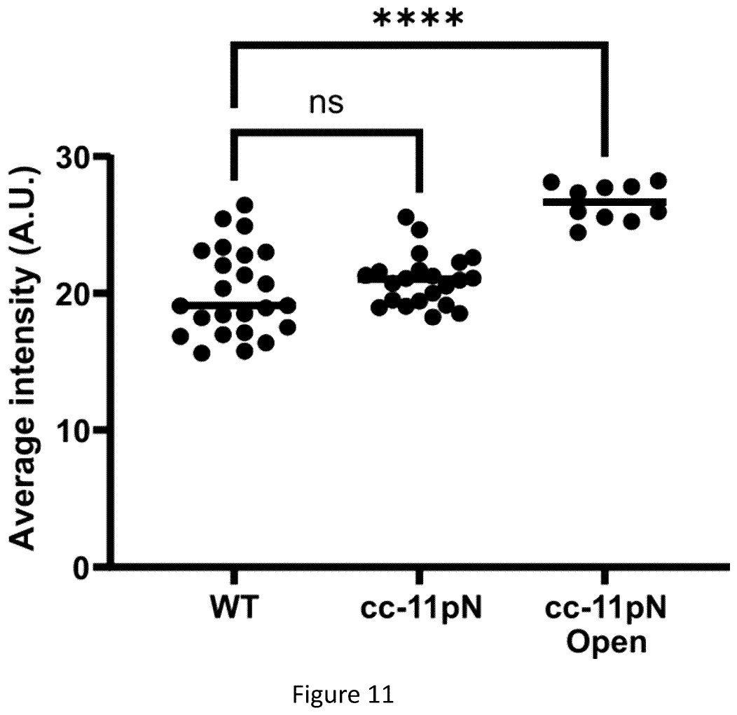

cc-11pN was inserted into the fission yeast homolog of Hip1R (End4p) at its genomic locus using CRISPR/Cas9 so that the expression level of End4p was not perturbed (FIG. 11). End4p functions as a dimer in vivo (Skruzny, M., et al., 2012, Proc Natl Acad Sci USA, 109:E2533-E2542; Abella, M., et al., 2021, Developmental Cell, 56:2419-2426.e4), and contains an IDR between its proline rich domain and its dimerization domain (FIG. 2). In wild-type cells, fluorescently tagged End4p is present at endocytic sites and appear as diffraction-limited puncta (hereafter referred to as End4p patches) that are enriched at cell tips during interphase and around the division plane during mitosis (FIG. 1F, FIG. 3A, FIG. 6B-C). When cc-11pN was inserted before the actin-binding THATCH domain of End4p, many large spherical End4p condensates appeared among small patches (FIG. 1G, FIG. 6E-F). The condensates remained visible in the cell during the entire for over 10 min whereas End4p remained at endocytic patches for a much shorter time (˜50 seconds). Interestingly, the condensates displayed liquid-like features such as fusion and fission (FIG. 1G, FIG. 3B-C).

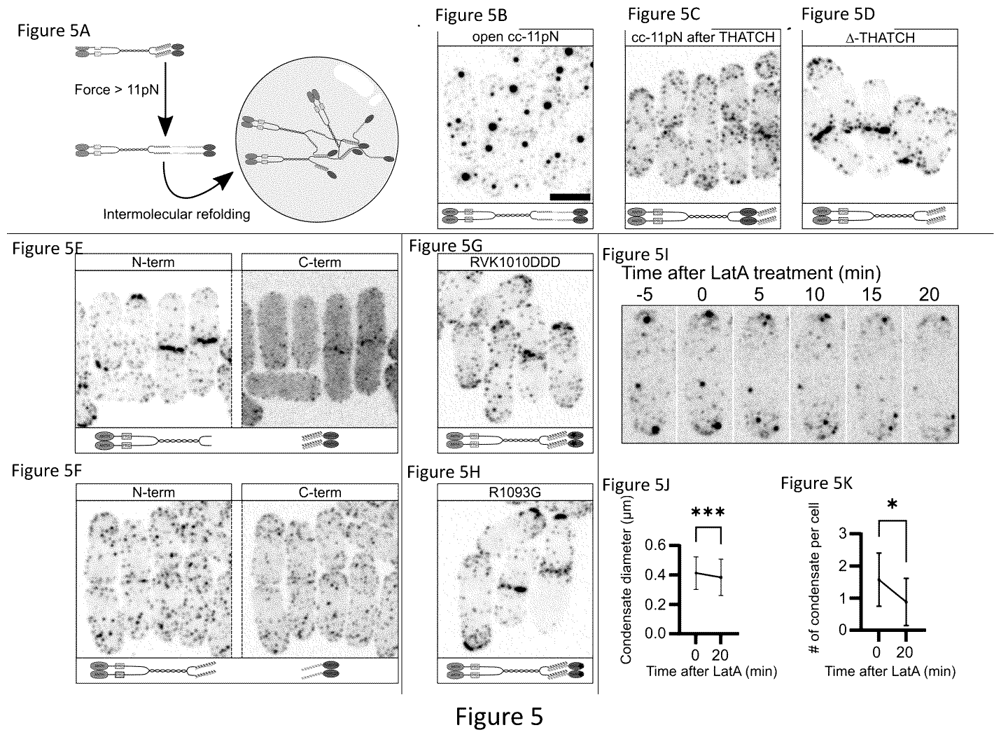

Condensate assembly of End4p constructs is mediated by force-induced unfolding of cc-11pN, which in turn leads to the formation of inter-molecular association of the unfolded α-helices (FIG. 5A, FIG. 4E). To test this, a construct with a shortened linker between the two α-helices of cc-11pN was employed to force the coiled-coil to be unfolded (Fig). Indeed, larger condensates formed (FIG. 5B, FIG. 6H-6I), supporting the fact that the unfolded coiled coils mediate the condensate assembly.

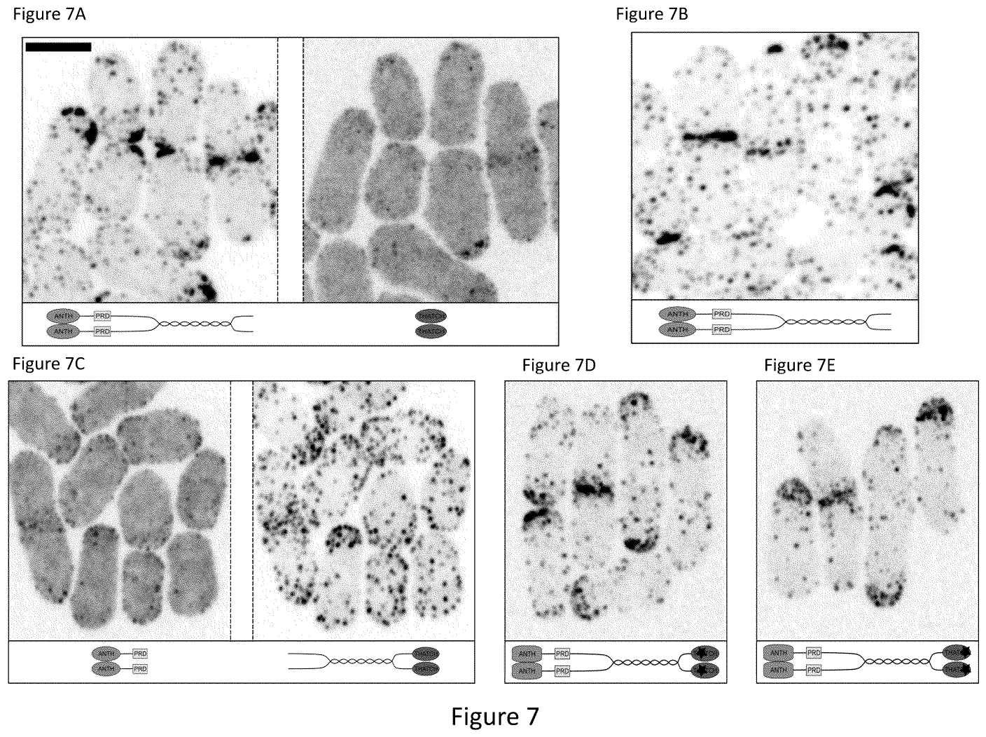

To further verify that the formation of End4p condensates is indeed force dependent, several controls were generated. To remove the force on cc-11pN, it was inserted at the C-terminal of End4p (FIG. 5C), the THATCH domain was deleted (FIG. 5D), or the construct was split after the dimerization domain of End4p using the self-cleaving 2A peptide (FIG. 5E). No condensates were observed in the three cases, suggesting that the incorporation of cc-11pN per se does not cause End4p condensation, and that force is needed to generate End4p condensates. In all these experiments, the cellular localization of these End4p constructs was the same with or without the insertion of cc-11pN (FIG. 7A-7B). Splitting cc-11pN between the two α-helices prohibited the formation of intermolecular coiled-coils, and thereby prevented the formation of End4p condensates (FIG. 5F, FIG. 4F). Mutations in the THATCH domain (RVK1010DDD in FIG. 5G, R1093G in FIG. 5H) that abolish the binding of End4p to F-actin also prevented the formation of End4p condensates (McCann, R. O., et al., 1997, Proc Natl Acad Sci USA, 94:5679-5684; Brett, T. J., et al., 2006, Nat Struct Mol Biol, 13:121-130). Here too it was confirmed that the insertion of cc-11pN did not change the localization of End4p (FIG. 7D-7E). Last, inhibiting the polymerization of F-actin through Latrunculin A (LatA), resulted in a reduction in both the size and the number of End4p condensates (FIG. 5I-5K), demonstrating that the formation of End4p condensates depends on F-actin polymerization. Collectively, the results indicate that force induced unfolding of cc-11pN in End4p promotes the phase separation of End4p constructs, and that the magnitude of force between End4p's dimerization and THATCH domain is above 11 pN.

Further investigation of the forces involved was accomplished by measuring force before a newly discovered domain in End4p that resembles R12 of talin (named Rend for R domain in End4p). Subsequently cc-14pN was inserted into End4p before the Rend domain. Examination of cells expressing this construct yielded condensates, indicating that the peak force applied before Rend is greater than 14 pN (FIG. 8A). Returning to End4p comprising a THATCH domain, an additional coiled-coil assembly, cc-18pN, was inserted before THATCH. Under the same conditions as above, formed once again, indicating that a peak force greater than 18 pN is applied before THATCH (FIG. 8B).

### Fine Measurement of Force and Measurement at Alternate Locations

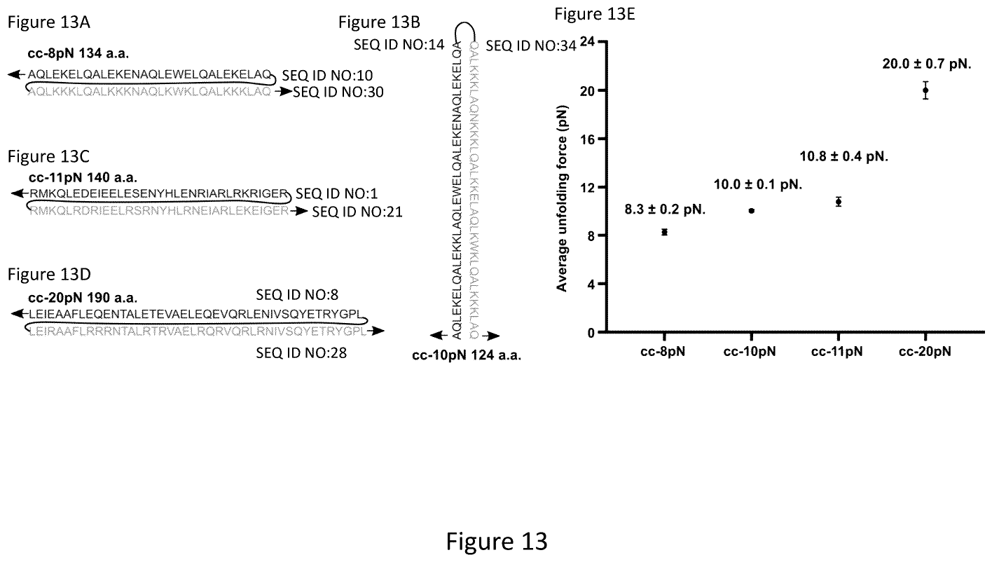

To further assess the force involved in the condensate assembly, three more force sensors were designed and tested (based on artificial coiled-coils (O'Shea, E. K., et al., 1993, Current Biology, 3:658-667; Gurnon, D. G., et al., 2003, J. Am. Chem. Soc., 125:7518-7519) and a B-ZIP protein (Moll, J. R., et al., 2001, Protein Sci, 10:649-655) (FIGS. 12A-12D) with average unfolding force thresholds of 8.2±0.2 pN, 10.0±0.1 pN and 20.0±0.7 pN, which are hereafter referred to as cc-8pN, cc-10pN and cc-20pN respectively (FIG. 13E, FIG. 14A-13C). The insertion of these three coiled-coils into actin-binding defective End4p did not lead to the formation of End4p condensates (FIGS. 13D-13F), and the insertion of these force sensors into the same position as cc-11pN showed that the magnitude of force before the THATCH domain is 11-20 pN (FIG. 9A, right column).

This library of calibrated coiled-coil force sensors not only allowed refinement of force measurements before the THATCH domain but measurement of the force at two other locations on End4p (FIG. 9A, FIG. 2A). The coiled-coil force sensors were inserted after the lipid-binding ANTH domain (FIG. 9A, left column), between the proline rich domain and the dimerization domain (FIG. 9A, middle column), or before the THATCH domain (FIG. 9A, right column). The insertion of the same coiled-coil at different locations led to End4p condensates in some, but not all, cases (compare each row in FIG. 9A). Because the formation of End4p condensates reflects the local presence of force above the unfolding force threshold of the coiled-coil force sensors, the results demonstrate a gradient in the magnitude of force along End4p molecule: between 8 and 10 pN after the ANTH domain, between 10 and 11 pN between the proline rich domain and the dimerization domain, and between 11 and 20 pN before the THATCH domain. These data also provide internal controls for the full functionality of the constructs when the forces are lower than the sensors' force thresholds, since the timing of endocytosis is unchanged for all constructs that do not condensate (FIG. 10).

Force from F-Actin is Relayed and Redistributed Through End4p

The gradient of force on End4p suggests that the binding of other endocytic coat proteins to End4p dissipates the force transmitted from F-actin to the lipid membrane. This was demonstrated by splitting End4p after its proline rich domain, therefore disconnecting End4p N-terminal from its C-terminal, but preserving the localization of End4p C-terminal at endocytic patches (FIG. 7C). Despite its inability to bind lipid, forces on End4p C-terminal were still large enough to unfold cc-11pN, confirming that forces are also transmitted to End4p via its binding partners in the endocytic coat (FIG. 9B). In the same vein, when the THATCH domain was mutated to prevent the direct binding between End4p and F-actin, forces higher than 8 pN were still measured between End4p's ANTH and proline rich domains, while forces after the proline rich domain were now smaller than 8 pN (whereas they were larger than 8 pN in wild-type cells) (FIG. 9C-9D). This result clearly shows that other endocytic coat proteins mediate the transmission of force to the N-terminal of End4p, even when End4p itself is unable to bind F-actin. Consequent to the extensive inter-connectivity of the endocytic coat, the deletion of the F-actin binding domain of another putative force transmitting endocytic protein Ent1p (homologous to Human epsin-1) only led to an increase of tension on End4p before the THATCH domain, while forces after the ANTH domain or after the proline rich domain remain unchanged (FIG. 12). Taken together, these results demonstrate that, contrary to the common belief, forces are not transmitted directly through adaptor proteins such as End4p but are relayed and redistributed by End4p and its binding partners along the different layers of the endocytic coat.

The materials and methods used are described herein.

### Protein Constructs and Purification

The codon optimized DNA of coiled-coils were synthesized (Invitrogen) and cloned into pGEX-6P-1 vectors (Sigma-Aldrich) after PCR amplification and Gibson cloning (New England BioLabs). Constructs were confirmed by DNA sequencing and introduced into BL21 (DE3) (New England BioLabs) for protein expression. GST fusion proteins were purified by binding to Glutathione Sepharose 4B beads (GE Healthcare) and the GST tag was removed by cleaving with PreScission Protease (Sigma-Aldrich). The purified proteins were exchanged to the biotinylation buffer containing 25 mM HEPES, 200 mM potassium glutamate with pH 7.7 and biotinylated at the Avi-tag in the presence of 50 μg/mL BirA, 50 mM bicine buffer, pH 8.3, 10 mM ATP, 10 mM magnesium acetate, and 50 μM d-biotin (Avidity) at 4° C. overnight.

### Protein-DNA Handle Crosslinking

The PCR-generated DNA handle used in the single-molecule experiments was 2,260 bp in length and contained a thiol group (—SH) at one end and two digoxigenin moieties at the other end. The DNA handle was crosslinked to the coiled coil protein construct as was described previously (Gao, Y., et al., 2011, J. Am. Chem. Soc., 133:12749-12757). Briefly, the protein construct was mixed with the DTDP-treated DNA handle in a 50:1 molar ratio in 100 mM phosphate buffer, 500 mM NaCl, pH 8.5 and incubated at room temperature overnight.

### Single-Molecule Manipulation Experiments

All pulling experiments were performed using dual-trap high-resolution optical tweezers as previously described (Gao, Y., et al., 2011, J. Am. Chem. Soc., 133:12749-12757; Xi, Z., et al., 2012, PNAS, 109:5711-5716). Briefly, an aliquot of the crosslinked protein-DNA mixture was mixed with 5 μL 2.1 μm diameter anti-digoxigenin antibody coated polystyrene beads (Spherotech) and incubated at room temperature for 15 min. Then the anti-digoxigenin coated beads and 0.5 μL 1.7 μm diameter streptavidin-coated beads were diluted in 1 mL PBS buffer (137 mM NaCl, 2.7 mM KCl, 8.1 mM Na₂HPO₄, 1.8 mM KH₂PO₄, pH 7.4) and were separately injected into the top and bottom channels of a homemade microfluidic chamber. Both channels were connected to the central channel with glass tubing. The two beads entering the central channel were caught in two optical traps and brought close to allow a single protein to be tethered between them. All manipulation experiments were carried out in the PBS buffer supplemented with the oxygen scavenging system (400 mg/mL glucose (Sigma-Aldrich), 0.02 unit/mL glucose oxidase (Sigma-Aldrich), and 0.06 unit/mL catalase). All single molecules were pulled and relaxed by increasing and decreasing, respectively, the trap separation at a speed of 10 nm/s. This speed was chosen because during clathrin-mediated endocytosis in yeast ˜100-nm long actin filaments are assembled in ˜10 seconds (Sirotkin, V., et al., 2010, Mol Biol Cell, 21:2894-2904; Berro, J., et al., 2010, MBoC, 21:2905-2915), corresponding to an average growth speed of 10 nm/s. Since actin dynamics is the main source of force during endocytosis, it is expected that endocytic proteins participating in force transmission to be under loads moving at ˜10 nm/s. The data was processed by MATLAB codes as were described elsewhere (Gao, Y., et al., 2011, J. Am. Chem. Soc., 133:12749-12757; Xi, Z., et al., 2012, PNAS, 109:5711-5716) and the unfolding forces were determined from the force-extension curves.

### Yeast Strains and Media

The S. pombe strains used in this study are presented in Supplemental Table S1. Strains were constructed using the method described in a previous publication (Fernandez, R., et al., 2016, Yeast, 33:549-557), and verified by sequencing of the colony PCR products. Fission yeast cells were grown in YE5S (Yeast Extract supplemented with 0.225 g/L of uracil, lysine, histidine, adenine and leucine), and imaged in EMM5S (Edinburgh Minimum media supplemented with 0.225 g/L of uracil, lysine, histidine, adenine and leucine). Yeast cells were grown at 32° C. under 200 rpm shaking overnight to reach exponential phase at OD_{595 nm} between 0.3 and 0.5.

### Microscopy

Cells were imaged on 25% gelatin pad at room temperature on a Nikon TiE inverted microscope (Nikon, Tokyo, Japan) with a CSU-W1 Confocal Scanning Unit (Yokogawa Electric Corporation, Tokyo, Japan) under a CFI Plan Apo 100×/1.45NA Phase objective (Nikon, Tokyo, Japan). Images were acquired with an iXon Ultra888 EMCCD camera (Andor, Belfast, UK). mEGFP tagged strains were excited with a 488-nm argon-ion laser and filtered by Spectra X with a single band pass filter 510/25. mScarlet-I tagged strains were excited with a 561-nm argon-ion laser and filtered by Spectra X with a single band pass filter 575/25. Fluorescent signals from the whole cell were collected with 21 optical sections separated by 0.5 μm, and max projected to create 2D images. The laser excitation and image acquisition settings for the same fluorophore (mEGFP or mScarlet-I) were the same for all strains imaged. Images were displayed and analyzed with the Fiji distribution of ImageJ (NIH, USA).

### LatA Treatment

Cells were washed with EMM5S and loaded into CellASIC microfluidics chambers (Y04C-02-5PK, Millipore-Sigma, Saint Louis, USA) before LatA (Thermo Fisher, MA, USA) treatment. The media exchange was controlled by the CellASIC ONIX2 microfluidics system (Millipore-Sigma, Saint Louis, USA) with a flow pressure of 4 psi. LatA was diluted in EMM5S to a final concentration of 100 μM. Fluorescent signals from the whole cell were collected with 21 optical sections each with 0.5 μm thickness, and stacks were projected with average intensity. Cells were imaged every 5 minutes, and the final images were corrected for photobleaching (exponential fit) before being analyzed.

### Protein Condensate Analysis

For the quantification of End4p protein condensates, an ImageJ plugin was created that identifies protein condensates and excludes End4p patches based on fluorescence intensity (900-65535), size (4-500 pixel) and circularity (0.2-1.0). This plugin shows good agreement with manual selection. Examples of automatically identified protein condensates are shown in FIG. 6.

### Example 2: Creation of a Library of Force-Sensitive Coiled-Coils

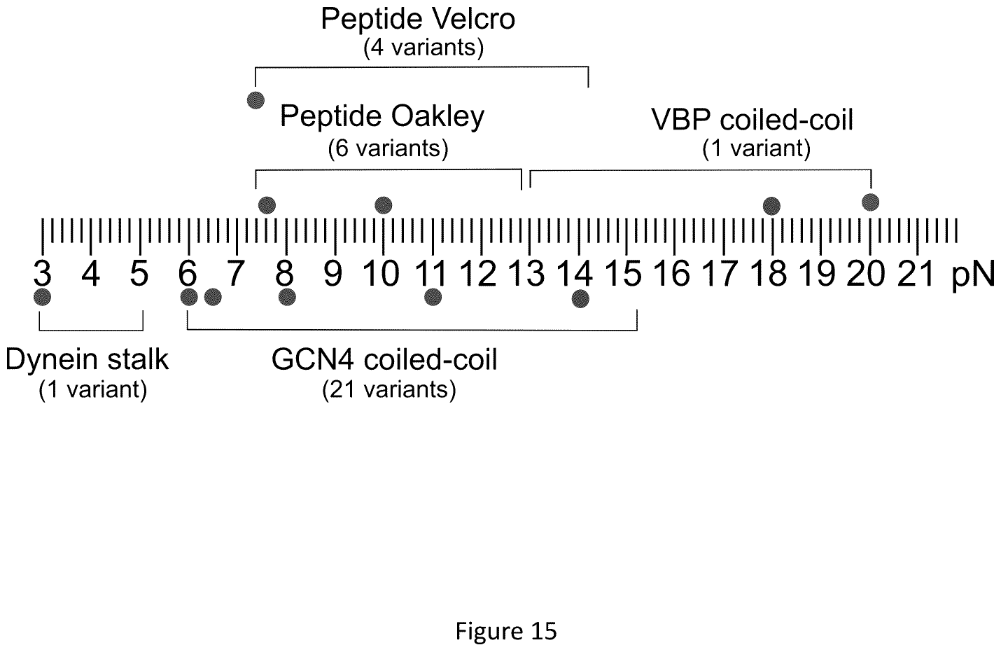

Here the existing set of coiled-coils is expanded to create a full library of coiled-coil force sensors that each unfold at a different threshold force, spanning the range of ˜3-20 pN (FIG. 15) (Ren, Y., et al., 2021, bioRxiv, 2021.06.29.450294). This range of force encompasses the majority of known force-producing biomolecules in vivo. Coiled-coils are developed with mean opening forces separated from each other by 1 pN or less. The library begins from several starting coiled-coil sequences for (GCN4, Peptide Oakley, Peptide Velcro, Dynein stalk, VBP). Rational mutation within the residues at the interface between the two α-helices and variance of the coiled-coils' length are employed, since extensive studies on coiled-coils have shown that the number of hydrophobic residues at this interface largely controls the affinity between α-helices. For a given coiled-coil base the force thresholds correlate with affinities, which is a trend that has been confirmed in the force sensors previously calibrated (Ren, Y., et al., 2021, bioRxiv, 2021.06.29.450294).

Following the protocols employed for the force sensors previously calibrated (FIG. 15 Error! Reference source not found.), the new coiled-coils are purified from E. coli and calibrated through in vitro pulling with optical tweezers (Ren, Y., et al., 2021, bioRxiv, 2021.06.29.450294). For each base design, preliminary data will be built upon to systematically create one positive and one negative control sensor that are respectively a) always unfolded (by removing the hydrophobic residues of the hydrophobic core or shortening the linker between α-helices), or b) always folded (by inducing the formation of disulfide bonds between cysteines introduced in the hydrophobic core).

The unfolding forces of the force sensors are not different in vitro and in vivo. First the pulling rate (i.e. the speed at which the force is applied on the protein) can significantly change the coiled-coil's opening force threshold, however the pulling rates in cells are in the same range as the pulling rates used in optical tweezer experiments (10 nm/s), and theory and experiments showed that unfolding forces increased by ˜20% when the pulling rate varies over 1-2 orders or magnitude (Xi, Z., et al., 2012, PNAS, 109:5711-5716; Goktas, M., et al., Chem Sci, 9:4610-4621).

It is not expected that insertion of the force sensors within proteins of interest will change their unfolding forces. FRET-based force sensors have been shown to work similarly in vivo and in vitro (Cost, A.-L., et al., 2015, Cel Mol Bioeng, 8:96-105; Grashoff, C., et al., 2010, Nature, 466:263-6). To ensure this is the case with the present force sensors, the optical tweezers are used to determine the unfolding force thresholds of select purified End4p constructs containing different sensors at different locations. The unfolding forces are the same for these constructs as for the sensors alone.

Several different coiled-coil bases that have overlapping force threshold ranges have been designed and will serve to cross-validate this strategy. Disulfide bonds are not typically formed in the cell's reducing environment, however if cysteines are maintained in close proximity (e.g., in a coiled-coil), a disulfide bond usually forms (Huang, H., et al., 2000, Chemistry & Biology, 7:355-364; He, C., et al., 2002, Chemistry & Biology, 9:1297-1303). Further, the force sensor with the highest force thresholds still provides a relatively good ‘folded’ control for most proteins of interest. To expand the sensor range to higher forces, protein domains with more complex folds and higher unfolding force thresholds are used (e.g., a previously characterized artificial SNARE complex construct) (Zorman, S., et al., 2014, Elife, 3:e03348; Gao, Y., et al., 2012, Science, 337:1340-3).

Data show that the unfolding force thresholds of coiled-coils have standard deviations ˜20% of the mean force (Ren, Y., et al., 2021, bioRxiv, 2021.06.29.450294). Currently, it remains unknown how these spreads can be rationally controlled by the amino acid sequence. Similarly, it remains unknown how the refolding forces are controlled. However, those properties do not have a large influence on in vivo measurements. The library contains enough coiled-coils with similar mean opening force but with different standard deviations and different refolding forces. By systematically introducing these sensors between different domains of End4p, the influence of the sensor's opening force standard deviation and refolding forces on the ability to discern the protein's force load is elucidated. In addition, the data identifies general rules that influence coiled-coil opening and refolding force distributions.

Not all locations within a protein are amenable to insertion of the force sensors. It is important not to insert the force sensors within known domains, instead it is preferred to insert them in linker regions between domains, avoiding residues predicted to be post-translationally modified or known motifs. Controls are included to ensure that the insertion of a force sensor does not affect the protein's function by using the always-folded version of the sensor and confirming proper function of the protein and the subcellular process it participates in. Since domain boundaries are not always unambiguously known, the insertion sites are moved by a few residues if the first insertion attempt is unsuccessful. This is confirmed by inserting “always-open” positive control sensors.

In general, the native interactions between domains of a POI do not interfere with the force measurements. False negative cases are imaginable where a coiled-coil force sensor is inserted between two domains of a POI and does not open even though those domains are in fact under a force higher than the coiled-coil force threshold. If those domains strongly interact with each other and the force to separate them is larger (e.g., 7 pN) than the coiled-coil force threshold (e.g., 3 pN), the domains could be under a force (e.g., 6 pN) larger than the force sensor' threshold while the sensor remains closed. To limit this issue as much as possible, the protein's structure will be used when it is available, or its prediction, to determine the locations to insert the force sensors that do not disrupt the protein's domains, and are not between two domains that interact strongly. However, if it is required to insert force sensors at these locations (e.g., the domains are strongly suspected to be under force), it is important not to interpret negative results as the absence of force. As a control, force sensors with different thresholds are used and forces measured at different locations on the protein and on binding partners.

### Example 3: Intrinsic Fluorescence Readout of Force Sensors

In some data, the unfolding of the force sensors within End4p is detected thanks to the aggregation of these open constructs. However, this readout is not always ideal because aggregates of the POI constructs may not be easy to distinguish from the normal POI localization. This readout also lacks spatial and temporal resolution. In addition, force sensors with lower thresholds (<7 pN) do not aggregate easily, likely because the affinity between α-helices is too low to favor inter-molecular binding, which is central for aggregation, or the POI into which the coiled-coil sensor has been inserted lacks domains or residues prone to phase separation. Thus, the readouts of the force sensors are expanded by introducing a) a split-GFP or b) a conformation-sensitive fluorescent protein within the loop linking the α-helices of the sensor. These direct fluorescence readouts extend the potential applications of our force sensors by making them easier to use and add spatial and temporal resolution.

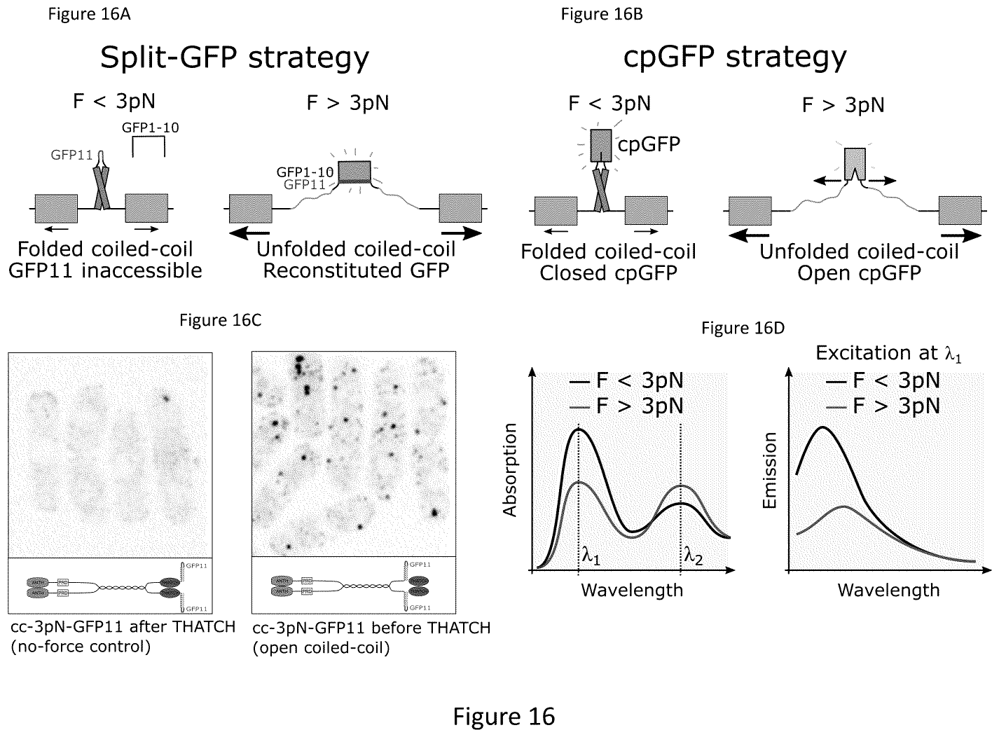

The “split-GFP strategy” is based on a previously engineered GFP that was split into two fragments, a short 16 amino-acid peptide (GFP11) and the rest of protein (GFP1-10), which are individually not fluorescent, but fluorescence can be detected if both constructs bind to each other (FIG. 16A) (Cabantous, S., et al., 2005, Nat Biotechnol, 23102-107). As such, the GFP11 peptide is introduced within the loop of the coiled-coil force sensors. When the force applied on the sensor is lower than its threshold force, the coiled-coil remains closed and GFP11 is not accessible to GFP1-10, therefore fluorescence cannot be detected. If the force is larger than the threshold, the sensor opens and exposes GFP11 which binds to GFP1-10. This strategy is particularly amenable to antiparallel coiled coils (FIG. 16A).

As proof of concept, a 3 pN GFP₁₁ sensor, cc-3pN-GFP11, was inserted into End4p before or after the THATCH domain (FIG. 16A). Observation of cells expressing the insertion construct and GFP₁₋₁₀ yielded no fluorescence when the coiled-coil was placed after the THATCH domain while fluorescence was observed when cc-3pN-GFP11 was inserted before THATCH (FIG. 16C).

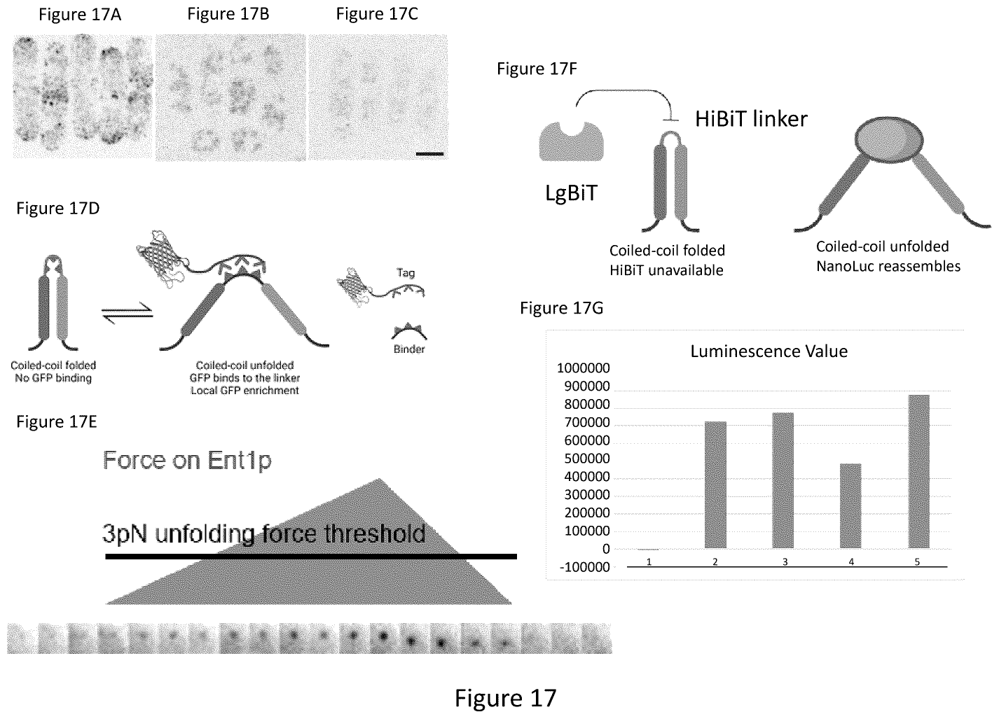

The split-GFP system was further examined in Ent1p, an additional protein involved in force redistribution during clathrin-mediated endocytosis. Three different force sensors were inserted into Ent1p, cc-3pN-GFP11, cc-5pN-GFP11, and cc-7pN-GFP11. Expression of the constructs in dividing yeast cells yielded no fluorescence in yeast expressing the cc-5pN-GFP11 and cc-7pN-GFP1 constructs, indicating that the peak force applied to Ent1p is less than 5 pN. Cells expressing the cc-3pN-GFP11 construct, however, yielded displayed fluorescence, indicating that the force applied to Ent1p is between 3 and 5 pN (FIG. 17A through FIG. 17C).

The second strategy (hereafter called “cpFP strategy”) is based on a circularly permuted fluorescent protein (cpFP) whose photophysical properties depend on its conformation. The cpFP conformational change modulates its absorption and emission spectra, which is directly monitored with a fluorescence microscope by measuring the emitted fluorescence intensity around its peak wavelength (e.g., 510 nm for cpEGFP) or the ratio of fluorescence emitted when excited at two absorption peak wavelengths (e.g., 400 nm and 490 nm for cpEGFP) (FIG. 16B). Flanked with protein domains that can bind a specific ligand (e.g., Ca²⁺, GTP, glutamate, etc.), cpFPs have been extensively used to develop numerous genetically-encoded biosensors to detect and quantify ions or metabolites in vivo (Germond, A., et al., 2016, Biophys Rev, 8:121-138; Kostyuk, A. I., et al., 2017, International Journal of Molecular Sciences, 20:4200; Nasu, Y., et al., 2021, Nature Chemical Biology, 17:509-518; Kroning, K. E., et al., 2021, Angewandte Chemie, 133:13470-13477; Sun, F., et al., 2018, Cell, 174:481-496.e19; Patriarchi, T., et al., 2018, Science, 360:eaat4422). Upon binding, the ligand binding domains change conformation which then propagates to the cpFP and changes its photophysical properties.

Using a similar idea, cpFPs are introduced into the linker region of the coiled-coil force sensors (FIG. 16). The unfolding of the coiled-coil when the force is larger than the threshold force will lead to a conformational change of the cpFP which will modulate its photophysical properties. This strategy is exemplified on two designs based on the GCN4-based parallel force sensor and the Oakley anti-parallel force sensor. The prototype fluorescent force sensors are inserted before the THATCH actin binding domain of End4p where they will be under force, thus unfolded, in standard conditions. End4p will also be tagged at its C-terminus with mScarlet-I to normalize the measurements to differences in expression levels and local concentrations. Treatment of the same cells with 100 μM Latrunculin A for 40 minutes stops actin assembly, consequently the forces on End4p, and the force sensor remains folded. This assay allows determination of the fluorescence properties of the sensors under high or no force and will be easy to scale up for the sensor's optimization. If necessary, the open coiled-coils under high force are mimicked by using variants of these sensors where all the hydrophobic residues at the interface between the α-helices are mutated to hydrophilic residues.

The circularly permuted green fluorescent protein (cpGFP) of the GCaMP6s calcium sensor is inserted in the linker region between the two α-helices of the force sensors (Kostyuk, A. I., et al., 2017, International Journal of Molecular Sciences, 20:4200). This cpGFP has a response amplitude of up to 100-fold in previously developed calcium sensors. Since the force sensors have very simple folds and are based on peptides that have previously been crystalized, structure prediction software (e.g., Alphafold, RoseTTa and RaptorX) allows rationally designed linkers between the α-helices and the cpGFP that best keep the coiled-coil and the cpGFP in closed conformations (Jumper, J., et al., 2012, Nature, 596:583-589; Simons, K. T., et al., 1997, Journal of Molecular Biology, 268:209-225; Baek, M., et al., 2021, Science, 373:871-876; Wang, S., et al., 2017, PLOS Computational Biology, 13:e1005324). Several initial designs are tested to select an optimal candidate. For each construct, using the in vivo assay mentioned in the previous paragraph, the intensity and ΔF/Fₘᵢₙ ratio are measured, where ΔF is the difference in fluorescence intensity between the open and closed versions of the fluorescent force sensors and Fₘᵢₙ is the fluorescence of the open version. Ideal initial designs have ΔF/Fₘᵢₙ between 0.25 and 1 for further optimization (Nasu, Y., et al., 2021, Nature Chemical Biology, 17:509-518).

From the same screen, the constructs with largest fluorescence differences when excited at 400 nm and 490 nm, hereafter referred to the 400/490 ratio, are identified (FIG. 16B). Such ratiometric sensors have the advantage to not require an extra fluorescent protein to normalize concentrations. In general ratiometric sensors are less bright and therefore more difficult to develop and to use (Barnett, L. M., et al., 2017, PLOS ONE, 12:e0170934).

These constructs are improved by rational screening of the residues closest to the cpGFP. First the amino acids of the coiled-coil linker region directly adjacent to the cpGFP “post” residues at position 145 and 148, as numbered in the GFP sequence, are screened (Nasu, Y., et al., 2021, Nature Chemical Biology, 17:509-518; Kroning, K. E., et al., 2021, Angewandte Chemie, 133:13470-13477; Sun, F., et al., 2018, Cell, 174:481-496.e19; Patriarchi, T., et al., 2018, Science, 360:eaat4422). The amino acid before the “144-post” by each of the other 19 amino acids are replaced, selecting the variant with highest ΔF/Fₘᵢₙ (or best 400/490 ratio), and repeating this strategy for the amino acid after the “148-post”. If necessary, this strategy is repeated for the next adjacent residues to further optimize the fluorescent sensors.

A third strategy for investigating the forces acting upon a protein of interest revolves around the binding of a tagged fluorescent protein to a binder peptide in the linker domain. Upon sufficient application of force, the binder peptide is exposed to its surroundings, allowing the tagged fluorescent protein to bind and produce an area of enriched fluorescence (FIG. 17D). When the force is removed from the protein, the coiled-coil reforms and the fluorescence signal drops. As proof of concept, a 3 pN coiled-coil-IAAL-K3 peptide construct was inserted into Ent1p and the fluorescence intensity was monitored in yeast cells over time. mEGFP-IAAL-E3 is recruited to the endocytic site only when force on Ent1p exceeds 3 pN (FIG. 17E).

An additional strategy, utilizing bioluminescence, revolves around the use of a split luciferase (NanoLuc). An HiBiT linker is inserted between the two alpha-helices, rendering it unable to form an enzymatically active NanoLuc. When the construct is expressed in the presence of LgBiT, and sufficient force applied to the coiled-coil, the two domains reassemble into an enzymatically active NanoLuc and catalyzes the conversion of furimazine into furimamide, yielding detectable luminescence (FIG. 17F). Insertion of a 3 pN coiled-coil HiBiT construct into several Ent1p variants further demonstrated the minimum force applied to the protein is 3 pN (FIG. 17G).

Once one GCN4- and one Oakley-based force sensor is optimized, other force sensors of the library are easily transformed into fluorescent ones with little to no further optimization, as they are all coiled-coils and have near identical structures. Since many force sensors differ by only a few residues from those two coiled-coils, fluorescent versions are virtually optimum if the same cpGFP and linkers are used. For force sensors based on other coiled-coils (Dynein-I, VBP and Velcro), the same insertion linkers are used as starting points and a similar optimization strategy repeated if necessary.

Previous measurements of forces on proteins using FRET-based force sensors provide candidates for validating force sensors. To test the full range of forces covered by the force sensors proteins involved in focal adhesions of mammalian cells are assessed (forces in the 1-10 pN range) (Freikamp, A., et al., 2016, Trends in Cell Biology, 26:838-847; Ringer, P., et al., 2017, Nature Methods, 14:1090-1096).

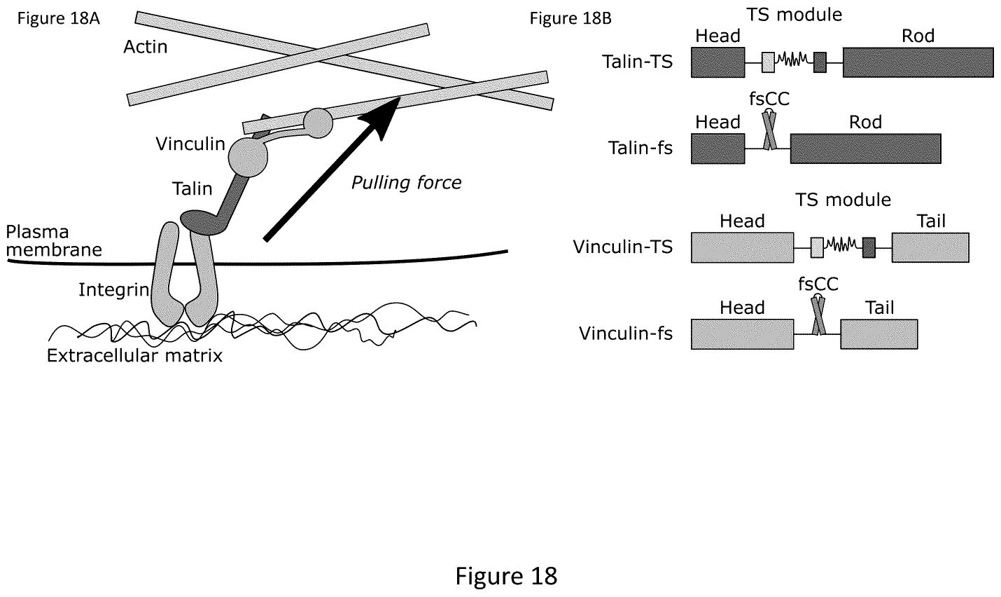

Focal adhesions (FAs) are mammalian cells' adhesion complexes that link the extra-cellular matrix (ECM) to the cell's actomyosin cytoskeleton and participate in the transmission of force between the cell and the ECM (FIG. 18). FRET-based force sensors have been used to measure forces on FA proteins talin and vinculin (respectively in the 7-10 pN and 1-6 pN range when FAs are engaged) (Grashoff, C., et al., 2010, Nature, 466:263-266; Austen, K., et al., 2015, Nature Cell Biology, 17:1597-1606). Therefore, FAs are an ideal system to a) validate the calibration of our force sensors, b) compare the ease of implementation of the force measurement strategy with strategies relying on FRET-based sensors, and c) demonstrate the strategy is applicable to mammalian cells. Plasmids expressing vinculin and talin constructs are modified to replace the FRET sensors with coiled-coil force sensors with thresholds in appropriate ranges (FIG. 18B), with or without the fluorescence reporters. The reduction of forces on talin and vinculin when the actomyosin contractility is disrupted using actin depolymerizing drugs (e.g., latrunculin A) or myosin inhibitors (e.g., blebbistatin) will be confirmed.

### Example 4: Identification of Proteins that Modulate the Forces within a Sub-Cellular Process

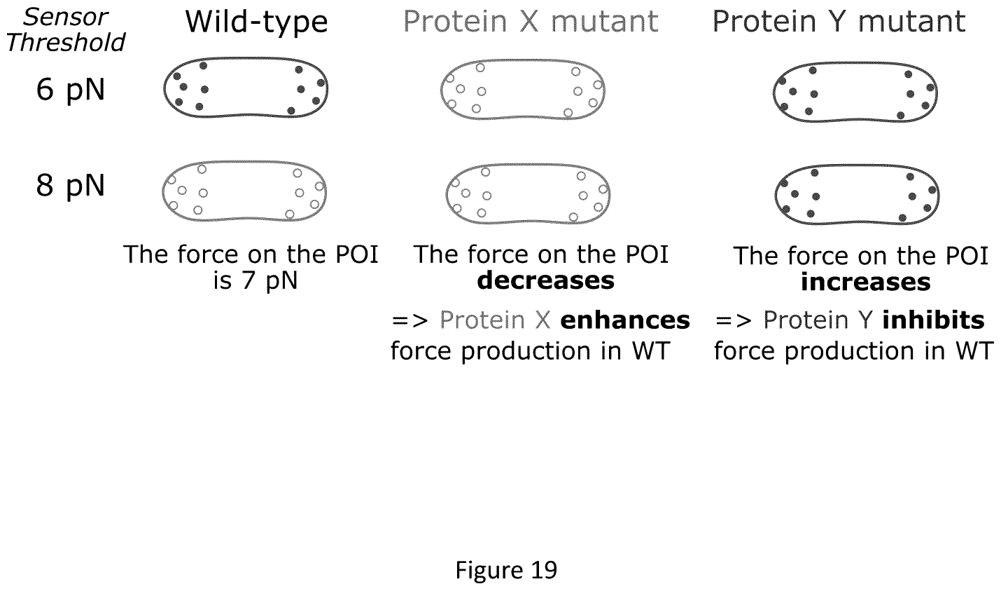

The set of force sensors allows discovery of proteins that modulate the forces produced within a subcellular process. Once a POI is known to be under force, by genetically, chemically or mechanically perturbing other proteins of the subcellular process, whether the force on the POI increases or decreases can be determined by monitoring the open or closed state of the force sensor it harbors (FIG. 19).

This approach can be applied to any sub-cellular process in any organism. Here, for validation of the method, the technique is applied to study forces during clathrin-mediated endocytosis (CME) in fission yeast. This system has many advantages: a) virtually all the CME proteins have been identified so the screening space is well defined and easily tractable, b) several proteins have been proposed to be involved in force production or transmission, and are good pilots for the method; yet, c) only one CME protein has been unambiguously demonstrated to bear force and e) a quantitative measurement or a relative contribution is unknown for all but one CME protein (Lacy, M. M., et al., 2018, FEBS Letters, 592:3586-3605; Ren, Y., et al., 2021, bioRxiv, 2021.06.29.450294; Goode, B. L., et al., 2015, Genetics, 199:315-58; Abella, M., et al., 2021, Developmental Cell, 56:2419-2426.e4). This approach is also extended to other organisms and other sub-cellular processes.

Cells expressing End4p constructs containing a force sensor with a 11 pN threshold (which aggregates in normal conditions) were treated with the actin polymerization inhibitor Latrunculin A (FIG. 5I-5K) (Ren, Y., et al., 2021, bioRxiv, 2021.06.29.450294). This treatment reverted the aggregation of the End4p constructs, indicating that the force on End4p is reduced to less than 11 pN when actin assembly is inhibited (Ren, Y., et al., 2021, bioRxiv, 2021.06.29.450294). This result corroborates the idea that actin assembly participates in generating the forces that are transmitted by End4p to deform the plasma membrane during CME.

In another experiment, it was observed that End4p constructs containing a force sensor with a 20 pN threshold near its actin binding domain (which does not aggregate in wild-type) aggregated after the actin binding domain of epsin Ent1p was deleted (Ren, Y., et al., 2021, bioRxiv, 2021.06.29.450294). This result demonstrates that Ent1p has a role in reducing the force from the actin meshwork transmitted to End4p and corroborates the idea that in wild-type Ent1p also bears actin forces and shares the load with End4p (Garcia-Alai, M. M., et al., Nature Communications, 9:328; Skruzny, M., et al., 2012, PNAS, 109:42; Skruzny, M., et al., 2015, Developmental Cell, 33:150-162; Lizarrondo, J., et al., 2021, Nat Commun, 12:2889).

It is now accepted that actin dynamics is important for efficient endocytosis in yeast and mammals, especially when the cell is challenged mechanically (high membrane tension or turgor pressure). However, how force is produced and transmitted during CME remains debated. The leading hypothesis in the field is that actin polymerization around the clathrin patch pushes inwards an entangled actin meshwork, which consequently pulls the membrane into a pit via adaptor proteins. However, the estimated forces produced by polymerization only are one to two orders of magnitude too small (Lacy, M. M., et al., 2018, FEBS Letters, 592:3586-3605; a, R., et al., 2021, Biophysical Journal, 120:1625-1640; Dmitrieff, S., et al., 2015, PLoS Comput Biol, 11:e1004538; Dmitrieff, S., et al., 2016, J Cell Biol, 212:763-766; Berro, J., et al., 2014, MBoC, 25:3515-3527; Berro, J., et al., 2010, MBoC, 21:2905-2915). There is therefore a need to identify other proteins that contribute to force production. In addition, the mechanisms of force transmission are more complex than previously thought since the data demonstrate forces are collected and redistributed by multiple proteins of the endocytic machinery before they are applied to the membrane.

To identify the proteins that modulate the forces applied to the plasma membrane the effect of full deletion, domain deletion, or point mutations on CME proteins is determined in cells expressing End4p constructs containing the 8 or 10 pN force sensors just after its ANTH lipid binding domain (respectively called ANTH-cc_{8pN}-End4p and ANTH-cc_{10pN}-End4p). Since ANTH-cc_{8pN}-End4p aggregates in wild-type cells, if aggregates are not observed when protein X is mutated, protein X impedes force production or force transmission to the membrane. Conversely, since ANTH-cc_{10pN}-End4p does not aggregate in wild-type cells, if aggregates are observed if protein Y is mutated, protein Y enhances force production or transmission (FIG. 19).

A library of mutants is constructed using scarless and markerless CRISPR-Cas9 (Fernandez et al., 2016, Yeast, 33(10):549-557). Building this strain necessitates a single simple transformation, since cells selected for the plasmid expressing Cas9 and the guide RNA have high chances to eventually harbor the intended edits (confirmed by sequencing). Since fission yeast lose plasmids when the selection pressure is removed, the same protocol and plasmid selection is reused to introduce one of the force sensors in each mutant of the library in one transformation. This step is multiplexed easily because it is the same transformation for all mutants. The initial library focuses on mutating proteins that contain an actin binding domain or motif (by domain deletion or point mutation to remove actin binding). The second focus is on proteins suspected to bind End4p (therefore good candidates to distribute forces from and to End4p) by domain deletion or point mutations. The approach is then extended to other CME proteins to discover new proteins that have not yet been shown to participate in force production or transmission during CME.

Fimbrin is a protein that crosslinks actin filaments. It is the second most abundant protein after actin at sites of CME. It is believed to be under high stress and directly participate in the transmission of forces during CME (Berro, J., et al., 2014, MBoC, 25:3515-3527; Picco, A., et al., 2018, MBoC, 29:1346-1358; Planade, J., et al., 2019, PLOS Biology, 17:e3000500; Ma, R., et al., 2019, Cytoskeleton, 76:346-354; Ma, R., et al., 2018, PLOS Computational Biology, 14:e1006150). Therefore, it is an excellent candidate as a reporter of the stresses generated within the actin meshwork.

First, the library of force sensors is used to determine the tension between fimbrin domains. Fimbrin has an EF-hand (EH) domain and two actin binding domains, ABD1 and ABD2. Previous data suggest that all three domains participate in actin binding (not just ABD1 and ABD2 as commonly believed). The force sensor library is used with the split-GFP reporter in the loop to measure the forces on fimbrin, since the forces on fimbrin are likely lower than 8 pN.

Then, the mutant library generated is used to introduce force sensors with thresholds just below and just above the measured force, in order to determine in which mutant the stresses on fimbrin increase or decrease. A set of proteins is identified that partially overlaps with the proteins identified above. Proteins present only in the above dataset correspond to proteins that transmit force from the actin meshwork to the membrane, and proteins present in both datasets correspond to proteins directly involved in force production or their upstream regulators. Proteins only identified in the present dataset correspond to proteins involved in actin stresses that are not transmitted to the membrane via End4p, and therefore point to other pathways for force transmission to the membrane or other mechanisms that do not fit in the “push-pull” paradigm for force production during CME.

### Example 5: Mapping the Forces Applied on the Proteins of a Sub-Cellular Process

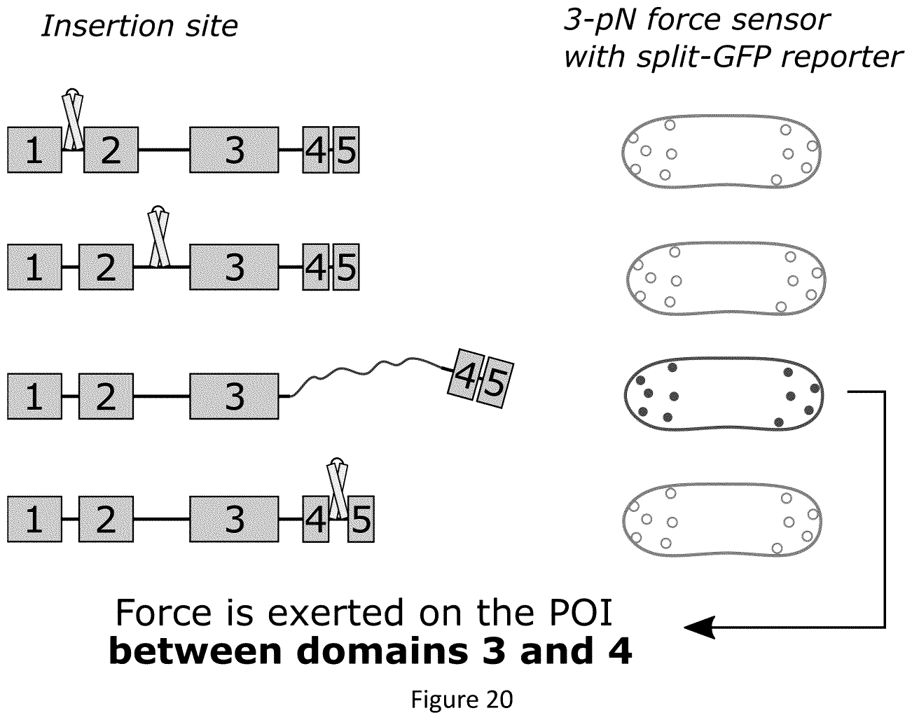

Upon inserting the weakest force sensor (˜3 pN) within a POI it is determined if POI is under force if a change is detected in the reporter state (either aggregation, appearance of GFP signal or change in the photophysical properties of the reporter, FIGS. 1-5, FIG. 9 and FIG. 16). Using a force sensor that is always folded, or that has the largest force threshold, allows for verification that a) insertion of a folded coiled-coil at the chosen site is innocuous and b) the change in the reporter state is indeed due to force (FIG. 15 and FIG. 20). By systematically using this pair of force sensors on a set of POIs, proteins that are under force during their normal cellular function are identified. Beyond this, the location of the force applied on the POI is determined by systematically introducing these two force sensors between consecutive domains of the POI (FIG. 20) and the magnitude of the force measured with high precision by testing force sensors with increasing force thresholds (FIG. 15 & FIG. 20).

The approaches to determine which proteins of a sub-cellular process are under force and determine the location and magnitude of these forces are universal, but here CME is chosen because enough is known to allow prophetic examples that are likely to work, and enough remains unknown about forces in this process that the discoveries are impactful.

Many proteins identified in the previous two datasets are force bearing, especially proteins identified as modulators of forces on End4p but not on Fim1p. Therefore, the strategy focuses on those proteins, and extends to other proteins suspected to be under force because they contain known actin or membrane binding domains, or are known or putative interactors of these proteins. For each candidate, a test sensor (e.g., 3 pN threshold with the split-GFP reporter) and a control sensor (e.g., unopenable or 20 pN threshold) are introduced between two consecutive domains. Domains are identified from database annotations (e.g., Uniprot or Pombase) from published (PDB) or predicted (AlphaFold) structures. The control sensors allow determination whether the insertion site is innocuous for the localization of the protein, which is similar to wild-type, and does not perturb endocytosis, as measured by FM4-64 dye internalization. Insertion sites within predicted unstructured regions are chosen and regions that contain known interacting motifs or residues prone to post-translational modification are avoided. Initially, sensors are not inserted within long intrinsically disordered regions because they potentially mediate phase separation, but force within these regions is probed in follow up studies when relevant. Sites adjacent to actin or membrane binding domains are first examined. When protein is indeed under force, the strategy and insert sensors between consecutive domains will be utilized to localize the force along the protein.

Once it is verified that force is applied at a location along a protein, the collection of force sensors with the split-GFP reporter is utilized to precisely measure the forces at those locations. The forces on the proteins identified are systematically measured to map the forces within the CME machinery. Proteins that have actin or membrane binding domains are prioritized, followed by known or putative partners for these proteins. The same strategy as above is used to narrow down the roles of protein domains in force transmission and identify or confirm the mechanical relationships between CME partners. Finally, measurements on select proteins are refined using the force sensors with the cpGFP reporter to determine at which stage of CME forces of different magnitudes are produced.

### Example 6: Utilization in Multicellular Organisms

Use of force sensors in unicellular organisms such as yeast provides insight into certain cellular mechanisms but does not shed significant light on how such forces affect the development of humans or other multicellular organisms. Therefore, the force sensors developed were used for experimentation in one Caenorhabditis elegans. As a transparent multicellular organism, C. elegans is an excellent model for optical force sensors. C. elegans additionally has the benefit of having one of the most well-studied developmental pathways.

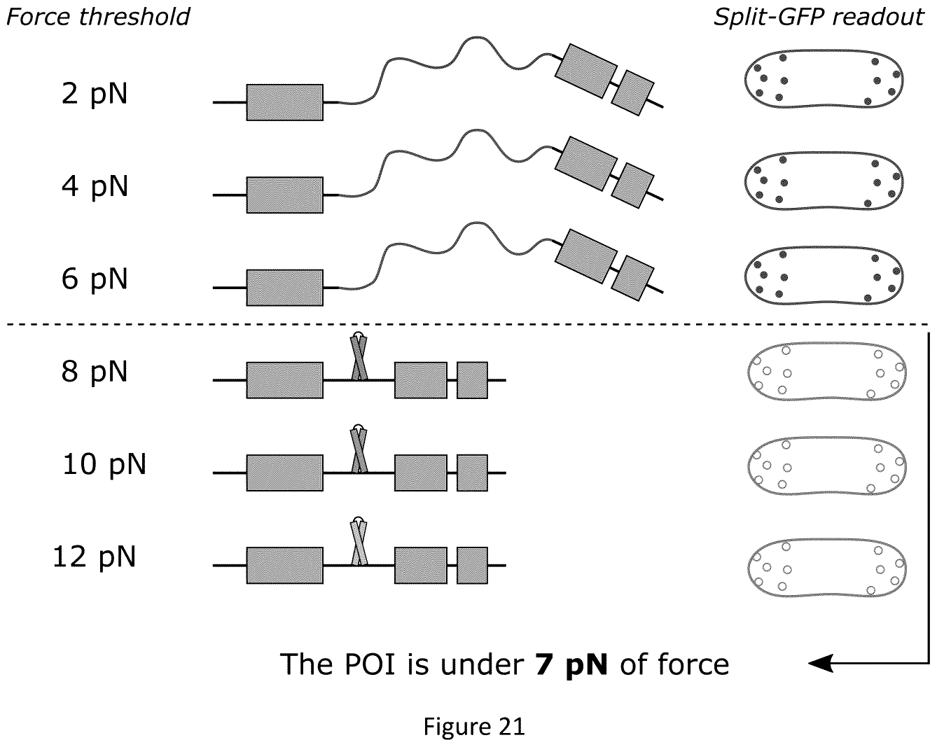

The C. elegans UNC-70 protein, encoded by the unc-70 gene, was selected as the first example, as it is homologous to human spectrin beta. To examine the forces acting on UNC-70, a variety of strains were obtained with various insertions between 1166R and 1167D (Table 1). Each of the strains was grown to the L4 larval stage, at which point they were mounted on 10% agarose patches and paralyzed in a droplet of 10 mM levamisole diluted in M9 medium for imaging (FIG. 21). Of particular interest in UNC-70 expression in C. elegans are the posterior lateral microtubule cells (PLM neurons, FIG. 21A) associated with the membrane-associated periodic skeleton (MPS/spectin lattice, FIG. 21), which are involved in sensation of force on the body and touch response. It was observed that in both wildtype and unc-115(ko) backgrounds, expression of the GFP₁₁ construct resulted in minimal GFP fluorescence. In contrast, animals of wildtype background expressing the 3 pN sensor construct exhibited significant GFP fluorescence, while those expressing the 10 pN construct yielded virtually no fluorescence, indicating that UNC-70 experiences between 3 and 10 pN of force in the MPS. Of interest is the observation that unc-115(ko) animals expressing the 3 pN sensor construct did not exhibit an increase in fluorescence. As the UNC-115 protein is required for actin binding and periodic MPS stability, in its absence no forces act upon UNC-70, resulting in a lack of fluorescence.

TABLE 1
C. elegans force sensor strains

| | | | DNA Insert | AA Insert |
|---|---|---|---|---|
| Strain | Background | Insert | SEQ ID NO | SEQ ID NO |
| Phx7438 | Wildtype | GFP₁₁ | 81 | 46 |
| Phx7460 | Wildtype | cc-3pN- | 82 | 79 |
| | | GFP₁₁ | | |
| Phx7442 | Wildtype | cc-3pN- | 83 | 80 |
| | | GFP₁₁ | | |
| MTS2376 | unc-115(ko) | cc-3pN- | 82 | 79 |
| | | GFP₁₁ | | |
| MTS2377 | unc-115(ko) | GFP₁₁ | 81 | 46 |

### Microscopy

Images were acquired with a DMi8 inverted microscope (Leica) equipped with a VT-iSIM system (Biovision) and an ORCA-Flash 4.0 camera (Hamamatsu). The microscope was controlled by the MetaMorph Advanced Confocal Acquisition Software. Images were acquired with an HC PL APO 40×/1.30NA OIL CS2 objective at a 488 nm laser line. Raw images were processed and analyzed in Fiji/ImageJ v2.3.0/1.53f51 (Schneider, C. A., et al., 2012, Nat Methods, 9, 671-675; Schindelin, J., et al., 2012, Nat Methods, 9, 676-682). Images were acquired in single layers and then stacked into maximum projections. To capture the intensity signal along the entire length of PLM, which could not be acquired in a single field of view, multiple images along the length of the neurite were taken and stitched into a single image using the pairwise-stitching plugin with a linear blending fusion method (Preibisch, S., et al., 2009, Bioinformatics, 25, 1463-1465).

### Fluorescence Quantification

To determine the mean fluorescence along the PLM neurite, a 5 pxl thick line was drawn along the center of the neurite and a signal intensity profile was generated by using the plot profile function and the signal intensity was averaged. To subtract background flourescence, the same signal intensity profile was acquired by shifting the drawn line from the center of the neurite by a few pxl into the non-neuronal tissue directly contacting the neurite. The signal intensity was calculated in arbitrary units as Iₘₑₐₙ=Iₘₑₐₙ (neurite)−Iₘₑₐₙ (background).

### Statistical Evaluation

Statistical evaluation was performed with GraphPad Prism (version 7). The dataset was first tested for normality distribution by using the D'Agostino and Pearson test to judge the use of non- vs parametric statistical tests.

### Example 7: Sequences of Force Sensor Proteins

Provided below are the sequences of exemplary force sensing peptides, along with their unfolding force threshold when available. Linkers are added before and after each coiled-coil to allow changes in coiled-coil orientation after insertion into target proteins. All linkers (before, between, and after alpha-helices) are underlined. Alpha-helices are in bold.

1. Alpha-Helix Sequences:
SEQ ID NO: 1
RMKQLEDEIEELESENYHLENRIARLRKRIGER
SEQ ID NO: 2
RMKQLEDEVEELESENYHLENRIARLRKRIGER
SEQ ID NO: 3
RSKQLEDEVEELESENYHLENRVARLRKRVGER
SEQ ID NO: 4
RMKQLEDEVEELESENYHLENRVARLRKRVGER
SEQ ID NO: 5
RMKQLEDKIEELLSKIYHLENEIARLKKLIGER
SEQ ID NO: 6
RSKQLEDEVEELESENYHLENRVAALRKRVGER
SEQ ID NO: 7
RMKQLEDEIEELESEIYHLENRIARLRKRIGER
SEQ ID NO: 8
LEIEAAFLEQENTALETEVAELEQEVQRLENIVSQYETRYGPL
SEQ ID NO: 9
LEIEAAFLEQENTALETEVAELEQEVQRLENEVSQYETRYGPL
SEQ ID NO: 10
AQLEKELQALEKENAQLEWELQALEKELAQ
SEQ ID NO: 11
AQLEKELQALEKENAQLEWELQALEKELAQLEKELQA
SEQ ID NO: 12
AQLEKELQALEKENAQLEWELQALEKELAQLEKELQALEKELAQ
SEQ ID NO: 13
AQLEKELQALEKKLAQLEWENQALEKELAQ
SEQ ID NO: 14
AQLEKELQALEKKLAQLEWELQALEKENAQLEKELQA
SEQ ID NO: 15
AQLEKEVQALEKKVAQLEWENQALEKEVAQ
SEQ ID NO: 16
AQSEKEVQALEKKVAQLEWENQALEKEVAQ
SEQ ID NO: 17
AQSEKEVQALEKKVAQLEWENQALEKESA
SEQ ID NO: 18
AQLEKELQALEKKLAQLEWENQALEKELAQLEKELQALEKELAQ
SEQ ID NO: 19
AQLEKELQALEKKLAQLEWENQALEKELAQLEKELQALEKELAQLEKELQALE
KELAQ
SEQ ID NO: 20
LRKIKETVDQVEELRRALRIKSQELEVKNAAANDKLKKMVKDQQEAEKKKVMS
QEIQEQLHKQQEVIADKQMSVKEDLDKVE
SEQ ID NO: 21
RMKQLRDRIEELRSRNYHLRNEIARLEKEIGER
SEQ ID NO: 22
RMKQLRDRVEELRSRNYHLRNEIARLEKEIGER
SEQ ID NO: 23
RSKQLRDRVEELRSRNYHLRNEVARLEKEVGER
SEQ ID NO: 24
RMKQLRDRVEELRSRNYHLRNEVARLEKEVGER
SEQ ID NO: 25
RMKQLEDKIEELLSKIYHLENEIARLKKLIGER
SEQ ID NO: 26
RSKQLRDRVEELRSRNYHLRNEVAALEKEVGER
SEQ ID NO: 27
RMKQLRDRIEELRSRIYHLRNEIARLEKEIGER
SEQ ID NO: 28
LEIRAAFLRRRNTALRTRVAELRQRVQRLRNIVSQYETRYGPL
SEQ ID NO: 29
LEIRAAFLRRRNTALRTRVAELRQRVQRARNRVSQYRTRYGPL
SEQ ID NO: 30
AQLKKKLQALKKKNAQLKWKLQALKKKLAQ
SEQ ID NO: 31
AQLKKKLQALKKKNAQLKWKLQALKKKLAQLKKKLQA
SEQ ID NO: 32
AQLKKKLQALKKKNAQLKWKLQALKKKLAQLKKKLQALKKKLAQ
SEQ ID NO: 33
AQLKKKLQANKKELAQLKWKLQALKKKLAQ
SEQ ID NO: 34
QALKKKLAQNKKKLQALKKELAQLKWKLQALKKKLAQ
SEQ ID NO: 35
AQVKKKLQANKKELAQVKWKLQAVKKKLAQ
SEQ ID NO: 36
AQVKKKLQANKKELAQVKWKLQAVKKKSAQ
SEQ ID NO: 37
AQSKKKLQANKKELAQVKWKLQAVKKKSAQ
SEQ ID NO: 38
AQLKKKLQALKKKLAQLKKKLQANKKELAQLKWKLQALKKKLAQ
SEQ ID NO: 39
AQLKKKLQALKKKLAQLKKKLQALKKKLAQLKKKLQANKKELAQLKWKLQA
LKKKLAQ
SEQ ID NO: 40
LRNELQKLEDDAKDNQQKANEVEQMIRDLEASIARYKEEYAVLISEAQAIKADLA
AVEAKVNRSTALLKSLSAERERWEKTS
2. Linker Sequences:
SEQ ID NO: 41
GGGGGGATNFSLLKLAGDVELNAGAGGGGG
SEQ ID NO: 42
GGGGGGGGGGGATNFSLLKLAGDVELNAGAGGGGGGGGGG
SEQ ID NO: 43
GGGSGGGSGGGSGGGSGGGSGATNFSLLKLAGDVELNAGAGGGSGGGSGGGSGGGS
GGGS
SEQ ID NO: 44
GGSSGG
SEQ ID NO: 45
GGGGG
3. Functional linkers:
GFP11: to reassemble with GFP1-10 to have fluorescence.
SEQ ID NO: 46
GGSRDHMVLHEYVNAAGITSGG
FIB1: to increase protein condensate size by the intrinsically
disordered sequence.
SEQ ID NO: 47
MAYTPGSRGGRGGSRGGRGGFNGGRGGFGGGRGGARGGGRGGARGGRGGRGGAR
GGRGGSSGGRGGAKGG
Short linker (for parallel coiled-coils): to increase protein
condensate size by forcing the coiled-coil to be open.
SEQ ID NO: 48
GGGGGSSGGGGG
Rigid linker (for antiparallel coiled-coils): to increase protein
condensate size by forcing the coiled-coil to be open.
SEQ ID NO: 49
GGRQLLAEKRELEEKKRREEEKKREEEERERERAQRGG
IAAK-K3: For reversible binding to IAAK-E3
SEQ ID NO: 50
GGKIAALKEKIAALKEKIAALKEGG
IAAK-E3: For reversible binding to IAAK-K3
SEQ ID NO: 51
EIAALEKEIAALEKEIAALEK
HiBiT: to reassemble with LgBit to have luminescence.
SEQ ID NO: 52
GGVSGWRLFKKISGG
SsrA: for reversible binding with SspB.
SEQ ID NO: 53
AANDENY
TRAP peptide 1: for reversible binding with different
TRAP proteins of varying affinities (e.g., TRAP2 and TRAP4).
SEQ ID NO: 54
MEEVF
TRAP peptide 2: for reversible binding with different
TRAP proteins of varying affinities (e.g., TRAP2 and TRAP4).
SEQ ID NO: 55
MEEVW
TEV linker: for recognition by the TEV protease for
cleavage.
SEQ ID NO: 56
ENLYFQS
4. GCN4 series (parallel coiled-coil; modified from natural coiled-coil):
GCN4-pIL-I16N-5E3R-2A-Mut-GCN4-pIL-I16N-5R3E (pJB289)
Unfolding force threshold: 11 pN
SEQ ID NO: 57
GGGGRMKQLEDEIEELESENYHLENRIARLRKRIGERGGGGGGATNFSLLKLAGD
VELNAGAGGGGGRMKQLRDRIEELRSRNYHLRNEIARLEKEIGERGGGG
GCN4-pIL-I16N-5E3R-I9V-2A-Mut-GCN4-pIL-I16N-5R3E
SEQ ID NO: 58
GGGGRMKQLEDEVEELESENYHLENRIARLRKRIGERGGGGGGATNFSLLKLAGD
VELNAGAGGGGGRMKQLRDRIEELRSRNYHLRNEIARLEKEIGERGGGG
GCN4-pIL-I16N-5E3R-I9V-2A-Mut-GCN4-pIL-I16N-5R3E-19V (pJB295)
Unfolding force threshold: 8 pN
SEQ ID NO: 59
GGGGRMKQLEDEVEELESENYHLENRIARLRKRIGERGGGGGGATNFSLLKLAGD
VELNAGAGGGGGRMKQLRDRVEELRSRNYHLRNEIARLEKEIGERGGGG
GCN4-p1-5E3R-M2S-2A-Mut-GCN4-p1-5R3E-M2S (pJB290)
Unfolding force threshold: 6/9/14 pN
SEQ ID NO: 60
GGGGRSKQLEDEVEELESENYHLENRVARLRKRVGERGGGGGGATNFSLLKLAGD
VELNAGAGGGGGRSKQLRDRVEELRSRNYHLRNEVARLEKEVGERGGGG
GCN4-p1-5E3R-2A-Mut-GCN4-p1-5R3E-M2S
SEQ ID NO: 61
GGGGRMKQLEDEVEELESENYHLENRVARLRKRVGERGGGGGGATNFSLLKLAG
DVELNAGAGGGGGRSKQLRDRVEELRSRNYHLRNEVARLEKEVGERGGGG
GCN4-p1-5E3R-2A-Mut-GCN4-p1-5R3E (pJB296)
Unfolding force threshold: 6.5 pN
SEQ ID NO: 62
GGGGRMKQLEDEVEELESENYHLENRVARLRKRVGERGGGGGGATNESLLKLAG
DVELNAGAGGGGGRMKQLRDRVEELRSRNYHLRNEVARLEKEVGERGGGG
GCN4-pIL-2A_Mut_LONG-GCN4-pIL (pJB324)
SEQ ID NO: 63
GGGGRMKQLEDKIEELLSKIYHLENEIARLKKLIGERGGGGGGGGGGGATNFSLL
KLAGDVELNAGAGGGGGGGGGGRMKQLEDKIEELLSKIYHLENEIARLKKLIGER
GGGG
GCN4-p1-5E3R-M2S-R25A-2A-Mut-GCN4-p1-5R3E-M2S-R25A
SEQ ID NO: 64
GGGGRSKQLEDEVEELESENYHLENRVAALRKRVGERGGGGGGATNFSLLKLAGD
VELNAGAGGGGGRSKQLRDRVEELRSRNYHLRNEVAALEKEVGERGGGG
GCN4-pIL-5E3R-2A_Mut-GCN4-pIL-5R3E (pJB329)
Unfolding force threshold: 13.9 ± 0.4 pN
SEQ ID NO: 65
GGGGRMKQLEDEIEELESEIYHLENRIARLRKRIGERGGGGGGATNFSLLKLAGDV
ELNAGAGGGGGRMKQLRDRIEELRSRIYHLRNEIARLEKEIGERGGGG
5. pER series (parallel coiled-coil; modified from natural coiled-coil):
pER-E-GGGS-pER-R (pJB325)
Unfolding force threshold: 20 pN
SEQ ID NO: 66
GGGGLEIEAAFLEQENTALETEVAELEQEVQRLENIVSQYETRYGPLGGGSGGGSG
GGSGGGSGGGSGATNFSLLKLAGDVELNAGAGGGSGGGSGGGSGGGSGGGSLEIRA
AFLRRRNTALRTRVAELRQRVQRLRNIVSQYETRYGPLGGGG
pER-E-GGGS-pER-R-LA (pJB330)
Unfolding force threshold: 17.5 ± 0.7 pN
SEQ ID NO: 67
GGGGLEIEAAFLEQENTALETEVAELEQEVQRLENEVSQYETRYGPLGGGSGGGS
GGGSGGGSGGGSGATNFSLLKLAGDVELNAGAGGGSGGGSGGGSGGGSGGGSLEIR
AAFLRRRNTALRTRVAELRQRVQRARNRVSQYRTRYGPLGGGG
Velcro series (parallel coiled-coil; artificial);
Velcro-p1 (pJB321)
Unfolding force threshold: 7.5 ± 0.3 pN
SEQ ID NO: 68
GGGGAQLEKELQALEKENAQLEWELQALEKELAQGGGGGGATNFSLLKLAGDVE
LNAGAGGGGGAQLKKKLQALKKKNAQLKWKLQALKKKLAQGGGG
Velcro-p1-A5B5-LONG (pJB322)
SEQ ID NO: 69
GGGGAQLEKELQALEKENAQLEWELQALEKELAQLEKELQAGGGSGGGSGGGS
GGGSGGGSGATNFSLLKLAGDVELNAGAGGGSGGGSGGGSGGGSGGGSAQLKKKL
QALKKKNAQLKWKLQALKKKLAQLKKKLQAGGGG
Velcro-p1-A6B6-LONG (pJB323)
SEQ ID NO: 70
GGGGAQLEKELQALEKENAQLEWELQALEKELAQLEKELQALEKELAQGGGSG
GGSGGGSGGGSGGGSGATNFSLLKLAGDVELNAGAGGGSGGGSGGGSGGGSGGGSA
QLKKKLQALKKKNAQLKWKLQALKKKLAQLKKKLQALKKKLAQGGGG
6. Oakley series (antiparallel coiled-coil; artificial):
Oakley-p1 (pJB297)
Unfolding force threshold: 7.3 ± 0.8 pN
SEQ ID NO: 71
GGGGAQLEKELQALEKKLAQLEWENQALEKELAQGGSSGGAQLKKKLQANKKE
LAQLKWKLQALKKKLAQGGGG
Oakley p1-OnePlus (pJB298)
Unfolding force threshold: 9.95 ± 0.31 pN
SEQ ID NO: 72
GGGGAQLEKELQALEKKLAQLEWELQALEKENAQLEKELQAGGSSGGQALKKK
LAQNKKKLQALKKELAQLKWKLQALKKKLAQGGGG
Oakley-p1-LV (pJB334)
Unfolding force threshold: 7.5 ± 0.2 pN
SEQ ID NO: 73
GGGGAQLEKEVQALEKKVAQLEWENQALEKEVAQGGSSGGAQVKKKLQANKK
ELAQVKWKLQAVKKKLAQGGGG
Oakley-p1-LV-L3S (pJB337)
SEQ ID NO: 74
GGGGAQSEKEVQALEKKVAQLEWENQALEKEVAQGGSSGGAQVKKKLQANKK
ELAQVKWKLQAVKKKSAQGGGG
Oakley-p1-LV-L3S-V28S (pJB338)
SEQ ID NO: 75
GGGGAQSEKEVQALEKKVAQLEWENQALEKESAQGGSSGGAQSKKKLQANKKE
LAQVKWKLQAVKKKSAQGGGG
Oakley-p1-TwoPlus (pJB339)
SEQ ID NO: 76
GGGGAQLEKELQALEKKLAQLEWENQALEKELAQLEKELQALEKELAQGGSSG
GAQLKKKLQALKKKLAQLKKKLQANKKELAQLKWKLQALKKKLAQGGGG
Oakley-p1-FourPlus (pJB340)
SEQ ID NO: 77
GGGGAQLEKELQALEKKLAQLEWENQALEKELAQLEKELQALEKELAQLEKEL
QALEKELAQGGSSGGAQLKKKLQALKKKLAQLKKKLQALKKKLAQLKKKLQA
NKKELAQLKWKLQALKKKLAQGGGG
7. Dynein stalk series (antiparallel coiled-coil; modified from
natural coiled-coil)):
Dynein stalk-p1 (pJB158
Unfolding force threshold: 3 pN
SEQ ID NO: 78
GGGGLRKIKETVDQVEELRRALRIKSQELEVKNAAANDKLKKMVKDQQEAEKK
KVMSQEIQEQLHKQQEVIADKQMSVKEDLDKVEGGGGGLRNELQKLEDDAKDN
QQKANEVEQMIRDLEASIARYKEEYAVLISEAQAIKADLAAVEAKVNRSTALLKS
LSAERERWEKTSGGGG
8. Caenorhabditis elegans Sensors (A* indicates a silent mutation):
3 pN GFP Sensor
SEQ ID NO: 79
GGGGLRKIKETVDQVEELRRALRIKSQELEVKNAAANDKLKKMVKDQQEAEKK
KVMSQEIQEQLHKQQEVIADKQMSVKEDLDKVEGGSRDHMVLHEYVNAAGITSG
GLRNELQKLEDDAKDNQQKANEVEQMIRDLEASIARYKEEYAVLISEAQAIKADL
AAVEAKVNRSTALLKSLSAERERWEKTSGGGG
10 pN GFP Sensor
SEQ ID NO: 80
GGGGAQLEKELQALEKKLAQLEWELQALEKENAQLEKELQAGGSRDHMVLHEYVN
AAGITSGGQALKKKLAQNKKKLQALKKELAQLKWKLQALKKKLAQGGGG
GFP₁₁ Insertion into unc-70
SEQ ID NO: 81
AACTTCTCAATCAACACGCTGCCATCCGTGGTGGATCACGAGATCACATGGTTCTG
CACGAGTACGTTAACGCCGCGGGAATCACCTCGGGCGGAGAAGAA*ATTGACGGA
TACGCTGAGGATTACAAGAAGATGCGTGCAATGGGAGATCGTGTCAC
3 pN GFP Sensor Insertion into unc-70
SEQ ID NO: 82
AACTTCTCAATCAACACGCTGCCATCCGTGGCGGCGGCGGCCTGAGAAAGATAAA
AGAGACTGTCGATCAAGTAGAAGAGCTCAGAAGAGCTCTTCGTATCAAATCG
CAAGAGCTTGAAGTAAAAAATGCAGCGGCAAACGACAAACTCAAGAAGATGG
TGAAGGACCAACAAGAGGCCGAAAAAAAAAAAGTAATGTCACAAGAAATTCA
AGAGCAGCTTCATAAACAACAGGAAGTCATCGCTGACAAGCAGATGTCAGTG
AAAGAGGATTTAGACAAGGTAGAGGGTGGATCACGAGATCACATGGTTCTGCAC
GAGTACGTTAACGCCGCGGGAATCACCTCGGGCGGACTGCGTAACGAGCTTCAG
AAGTTGGAGGACGATGCTAAAGACAACCAACAAAAAGCTAACGAAGTAGAAC
AGATGATACGTGACTTAGAAGCCAGTATAGCGCGATACAAAGAGGAGTATGC
TGTTTTGATCTCAGAGGCGCAGGCTATAAAGGCAGACCTGGCGGCGGTCGAG
GCGAAAGTTAATAGAAGTACAGCCCTTCTTAAATCTCTTAGTGCCGAGCGTGA
GCGATGGGAGAAAACGAGTGGTGGAGGTGGAGAAGAA*ATTGACGGATACGCT
GAGGATTACAAGAAGATGCGTGCAATGGGAGATCGTGTCAC
10 pN GFP Sensor Insertion into unc-70
SEQ ID NO: 83
AACTTCTCAATCAACACGCTGCCATCCGTGGTGGTGGCGGAGCGCAATTGGAAAA
GGAACTTCAGGCACTGGAAAAAAAGCTGGCGCAACTTGAGTGGGAGCTCCAA
GCTCTGGAGAAAGAGAATGCCCAGCTGGAAAAGGAGCTCCAAGCTGGTGGAT
CACGAGATCACATGGTTCTGCACGAGTACGTTAACGCCGCGGGAATCACCTCGGG
CGGACAGGCTCTGAAGAAGAAGTTGGCGCAGAACAAGAAGAAACTCCAAGCG
TTAAAGAAAGAGCTTGCACAGTTAAAATGGAAGCTTCAAGCGCTCAAGAAGA
AATTAGCCCAAGGTGGCGGCGGAGAAGAA*ATTGACGGATACGCTGAGGATTAC
AAGAAGATGCGTGCAATGGGAGATCGTGTCAC
9. Binding Partners of Sensors
GFP1-10: To assemble with GFP11 to form fluorescent GFP.
SEQ ID NO: 84
SKGEELFTGVVPILVELDGDVNGHKFSVRGEGEGDATIGKLTLKFICTTGKLPVPWPTL
VTTLTYGVQCFSRYPDHMKRHDFFKSAMPEGYVQERTISFKDDGKYKTRAVVKFEGD
TLVNRIELKGTDFKEDGNILGHKLEYNFNSHNVYITADKQKNGIKANFTVRHNVEDGS
VQLADHYQQNTPIGDGPVLLPDNHYLSTQTVLSKDPNEK
LgBiT: To assemble with HiBiT or SmBIT to form
catalytically active NanoLuc.
SEQ ID NO: 85
MVFTLEDFVGDWEQTAAYNLDQVLEQGGVSSLLQNLAVSVTPIQRIVRSGENALKIDI
HVIIPYEGLSADQMAQIEEVFKVVYPVDDHHFKVILPYGTLVIDGVTPNMLNYFGRPY
EGIAVFDGKKITVTGTLWNGNKIIDERLITPDGSMLFRVTINS
TRAP 1: To reversibly bind to TRAP peptides (e.g., MEEVF
and MEEVW).
SEQ ID NO: 86
MSYYHHHHHHDYDIPTTENLYFQGSKQALKEKELGNDAYKKKDFDTALKHYDKAKE
LDPTNMYILNQAAVYFEKGDYNKCRELCEKAIEVGRENREDYRLIAIAYARIGNSYFK
EEKYKDAIHFYNKSLAEHRTPKVLKKCQQAEKILKEQ
TRAP 2: To reversibly bind to TRAP peptides (e.g., MEEVF
and MEEVW).
SEQ ID NO: 87
MSYYHHHHHHDYDIPTTENLYFQGSKQALKEKELGNDAYKKKDFDTALKHYDKAKE
LDPTNMYIMNQAAVYFEKGDYNKCRELCEKAIEVGRENREDYRMIAYAYARIGNSYF
KEEKYKDAIHFYNKSLAEHRTPKVLKKCQQAEKILKEQ
TRAP 3: To reversibly bind to TRAP peptides (e.g., MEEVF
and MEEVW).
SEQ ID NO: 88
MSYYHHHHHHDYDIPTTENLYFQGSKQALKEKELGNDAYKKKDFDTALKHYDKAKE
LDPTNMYIMNQAAVYFEKGDYNKCRELCEKAIEVGRENREDYRMIAYAYADIGDSYF
KEEKYKDAIHFYNKSLAEHRTPKVLKKCQQAEKILKEQ
TRAP4: To reversibly bind to TRAP peptides (e.g.,
MEEVF and MEEVW).
SEQ ID NO: 89
MKQALKEKELGNDAYKKKDFDTALKHYDKAKELDPTNMTYIINQAAVYFEKGDYN
KCRELCEKAIEVGRENREDYRWIAIAYARIGNSYFKEEKYKDAIHFYNKSLAEHRTPK
VLKKCQQAEKILKEQ
SspB: To reversibly bind to SsrA.
SEQ ID NO: 90
SQLTPRRPYLLRAFYEWLLDNQLTPHLVVDVTLPGVQVPMEYARDGQIVLNIAPRAV
GNLELANDEVRFNARFGGIPRQVSVPLAAVLAIYARENGAGTMFEPEAAYD

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A force sensing peptide comprising two α-helix domains linked by a linker sequence wherein the two α-helix domains form a coiled coil domain, referred to as a closed conformation, in the absence of external force, and further wherein the coiled coil domain undergoes a conformational change to an open conformation in the presence of a force above an uncoiling threshold level for the coiled coil domain.

2. The force sensing peptide of claim 1, wherein the force sensing peptide is operably linked to a target molecule of interest, whereby the force sensing peptide undergoes the conformational change when force is applied to the target molecule of interest.

3. The force sensing peptide of claim 1, wherein the linker sequence comprises at least one functional domain or moiety for generating a signal when the force sensing peptide is in an open conformation.

4. The force sensing peptide of claim 3, wherein at least one functional domain or moiety is selected from the group consisting of a fluorescent protein or a fragment thereof, a circular fluorescent protein, a protein cleavage domain, an RNA binding molecule, a DNA-binding domain, an epitope for recognition by a binding molecule, an ion-binding domain, a lipid-binding domain, a peptide containing residues for post-translational modification, a peptide containing unnatural amino acids, a peptide that is a toxin to cells, a peptide with antibacterial activity, a peptide with anti-viral activity, a peptide with anti-fungal activity, a peptide with enzymatic activity, a peptide that modifies the enzymatic activity of other proteins, a peptide containing localization signal for cellular compartments, a peptide containing secretion signal, and a peptide that binds another peptide or protein construct.

5. The force sensing peptide of claim 1, wherein the uncoiling threshold level for the coiled coil domain is at least 3, 4, 5, 6, 7, 8, 9, 10, 11, 12, 13, 14, 15, 16, 17, 18, 19, 20, 21, 22, 23, 24, 25, or more than 25 piconewtons (pN).

6. The force sensing peptide of claim 1, wherein the peptide comprises at least one α-helix domain selected from the group consisting of SEQ ID NO:1-SEQ ID NO:40.

7. The force sensing peptide of claim 1, wherein the peptide comprises at least one set of α-helix domains selected from the group consisting of:
a) SEQ ID NO:1 and SEQ ID NO:21;
b) SEQ ID NO:2 and SEQ ID NO:21;
c) SEQ ID NO:2 and SEQ ID NO:22;
d) SEQ ID NO:3 and SEQ ID NO:23;
e) SEQ ID NO:4 and SEQ ID NO:23;
f) SEQ ID NO:4 and SEQ ID NO:24;
g) SEQ ID NO:5 and SEQ ID NO:25;
h) SEQ ID NO:6 and SEQ ID NO:26;
i) SEQ ID NO:7 and SEQ ID NO:27;
j) SEQ ID NO:8 and SEQ ID NO:28;
k) SEQ ID NO:9 and SEQ ID NO:29;
l) SEQ ID NO:10 and SEQ ID NO:30;
m) SEQ ID NO:11 and SEQ ID NO:31;
n) SEQ ID NO:12 and SEQ ID NO:32;
o) SEQ ID NO:13 and SEQ ID NO:33;
p) SEQ ID NO:14 and SEQ ID NO:34;
q) SEQ ID NO:15 and SEQ ID NO:35;
r) SEQ ID NO:16 and SEQ ID NO:36;
s) SEQ ID NO:17 and SEQ ID NO:37;
t) SEQ ID NO:18 and SEQ ID NO:38;
u) SEQ ID NO:19 and SEQ ID NO:39; and
v) SEQ ID NO:20 and SEQ ID NO:40,
wherein the set of α-helix domains are linked by a linker sequence.

8. The force sensing peptide of claim 1, wherein the peptide comprises at least one linker sequence selected from the group consisting of SEQ ID NO:41-SEQ ID NO:56.

9. The force sensing peptide of claim 1, wherein the peptide comprises a sequence selected from the group consisting of SEQ ID NO:57-SEQ ID NO:80.

10. The force sensing peptide of claim 2, wherein the target molecule of interest is selected from the group consisting of a detectable moiety, a fluorescent protein, a purification tag, a targeting domain, a cellular localization signal, a DNA molecule, an RNA molecule, cAMP, cGMP, eicosapentaenoic acid, eicosatetraenoic acid, a phosphatidylcholine, a phosphatidylinositol, a phosphatidylethanolamine, an aminomethyl polystyrene linker, a chloromethyl polystyrene linker, a PEG linker, a solid-phase protein synthesis resin, a C-terminal fragment of End4p, a C-terminal fragment of actin, a C-terminal fragment of clathrin, a C-terminal fragment of vinculin, a C-terminal fragment of talin, a C-terminal fragment of integrin, glycogen, cellulose, a therapeutic agent, an antibiotic, an antiviral, an anti-fungal, an anti-helminthic, an anti-inflammatory molecule, and a chemotherapeutic.

11. A system for determination of the level of force exerted on a target of interest, the system comprising at least two force sensing peptides of claim 1, where the at least two force sensing peptides have different uncoiling threshold levels.

12. A nucleic acid molecule encoding a force sensing peptide of claim 1.

13. A genetically modified host cell comprising a nucleic acid molecule of claim 12.

14. A method of detecting the force applied to a target molecule of interest, the method comprising contacting a target molecule of interest operably linked to a force sensing peptide of claim 1 with a sufficient level of force to induce a conformational change in the force sensing peptide, and detecting the presence of the open conformation of the force sensing peptide.

15. The method of claim 14, wherein the force sensing peptide comprises a linker sequence comprising at least one functional domain or moiety for generating a signal when the force sensing peptide is in an open conformation.

16. The method of claim 15, wherein at least one functional domain or moiety is selected from the group consisting of a fragment of a fluorescent protein, a bioluminescent protein, a circular fluorescent protein, a protein cleavage domain, an RNA binding molecule, a DNA-binding domain, an epitope for recognition by a binding molecule, an ion-binding domain, a lipid-binding domain, a peptide containing residues for post-translational modification, a peptide containing unnatural amino acids, a peptide that is a toxin to cells, a peptide with anti-bacterial activity, a peptide with anti-viral activity, a peptide with anti-fungal activity, a peptide with enzymatic activity, a peptide that modifies the enzymatic activity of other proteins, a peptide containing localization signal for cellular compartments, and a peptide containing secretion signal.

17. The method of claim 15, wherein the method of detecting comprises detecting a fluorescent signal that is generated in the presence of the open conformation of the force sensing peptide.

18. The method of claim 15, wherein the method of detecting comprises detecting a bioluminescent signal that is generated in the presence of the open conformation of the force sensing peptide.

19. The method of claim 15, wherein the method of detecting comprises detecting a differential level of a protein or an mRNA molecule when the force sensing peptide is in the open conformation.

20. The method of claim 15, wherein the method of detecting comprises detecting a change in localization of the target molecule due to cleavage of a peptide cleavage domain that is exposed when the force sensing peptide is in the open conformation.

21. The method of claim 14, wherein the peptide comprises at least one α-helix domain selected from the group consisting of SEQ ID NO:1-SEQ ID NO:40.

22. The method of claim 14, wherein the peptide comprises at least one set of α-helix domains selected from the group consisting of:
a) SEQ ID NO:1 and SEQ ID NO:21;
b) SEQ ID NO:2 and SEQ ID NO:21;
c) SEQ ID NO:2 and SEQ ID NO:22;
d) SEQ ID NO:3 and SEQ ID NO:23;
e) SEQ ID NO:4 and SEQ ID NO:23;
f) SEQ ID NO:4 and SEQ ID NO:24;
g) SEQ ID NO:5 and SEQ ID NO:25;
h) SEQ ID NO:6 and SEQ ID NO:26;
i) SEQ ID NO:7 and SEQ ID NO:27;
j) SEQ ID NO:8 and SEQ ID NO:28;
k) SEQ ID NO:9 and SEQ ID NO:29;
l) SEQ ID NO:10 and SEQ ID NO:30;
m) SEQ ID NO:11 and SEQ ID NO:31;
n) SEQ ID NO:12 and SEQ ID NO:32;
o) SEQ ID NO:13 and SEQ ID NO:33;
p) SEQ ID NO:14 and SEQ ID NO:34;
q) SEQ ID NO:15 and SEQ ID NO:35;
r) SEQ ID NO:16 and SEQ ID NO:36;
s) SEQ ID NO:17 and SEQ ID NO:37;
t) SEQ ID NO:18 and SEQ ID NO:38;
u) SEQ ID NO:19 and SEQ ID NO:39; and
v) SEQ ID NO:20 and SEQ ID NO:40,
wherein the set of α-helix domains are linked by a linker sequence.

23. The method of claim 14, wherein the peptide comprises at least one linker sequence selected from the group consisting of SEQ ID NO:41-SEQ ID NO:56.

24. The method of claim 14, wherein the peptide comprises at least one sequence selected from the group consisting of SEQ ID NO:57-SEQ ID NO:80.
